SEQUENCE LISTING (233 sequences)

SEQ ID NO: 1 - protein, 9 aa, Human papillomavirus 16
NLDTASTTL

SEQ ID NO: 2 - protein, 9 aa, Human papillomavirus 16
HDIILECVY

SEQ ID NO: 3 - protein, 11 aa, Human papillomavirus 16
TLQDVSLEVYL

SEQ ID NO: 4 - protein, 10 aa, Human papillomavirus 16
TIHDIILECV

SEQ ID NO: 5 - protein, 9 aa, Human papillomavirus 16
AFRDLCIVY

SEQ ID NO: 6 - protein, 15 aa, Human papillomavirus 16
KQRFHNIRGRWTGRC

SEQ ID NO: 7 - protein, 9 aa, Human papillomavirus 16
WPTTPPRPI

SEQ ID NO: 8 - protein, 9 aa, Human papillomavirus 16
YLHNRLVVF

SEQ ID NO: 9 - protein, 11 aa, Human papillomavirus 16
ALDGNLVSMDV

SEQ ID NO: 10 - protein, 11 aa, Human papillomavirus 18
TVLELTEVFEF

SEQ ID NO: 11 - protein, 9 aa, Human papillomavirus 18
SPATAFTVY

SEQ ID NO: 12 - protein, 19 aa, Human papillomavirus 16
VQSTHVDIRTLEDLLMGTL

SEQ ID NO: 13 - protein, 15 aa, Artificial Sequence
  Description of Artificial Sequence Synthetic peptide
CALSEGGGSQGNLIF

SEQ ID NO: 14 - protein, 16 aa, Artificial Sequence
  Description of Artificial Sequence Synthetic peptide
CASSPELAGPQETQYF

SEQ ID NO: 15 - protein, 11 aa, Artificial Sequence
  Description of Artificial Sequence Synthetic peptide
CAVSDRHDMRF

SEQ ID NO: 16 - protein, 14 aa, Artificial Sequence
  Description of Artificial Sequence Synthetic peptide
CASSYSPERHEQFF

SEQ ID NO: 17 - protein, 10 aa, Artificial Sequence
  Description of Artificial Sequence Synthetic peptide
CASEGHDMRF

SEQ ID NO: 18 - protein, 16 aa, Artificial Sequence
  Description of Artificial Sequence Synthetic peptide
CASSTEAGGPTGELFF

SEQ ID NO: 19 - protein, 14 aa, Artificial Sequence
  Description of Artificial Sequence Synthetic peptide
CIVRDRSYGQNFVF

SEQ ID NO: 20 - protein, 11 aa, Artificial Sequence
  Description of Artificial Sequence Synthetic peptide
CSAREGYRSYF

SEQ ID NO: 21 - protein, 14 aa, Artificial Sequence
  Description of Artificial Sequence Synthetic peptide
CARGLENAGNMLTF

SEQ ID NO: 22 - protein, 14 aa, Artificial Sequence
  Description of Artificial Sequence Synthetic peptide
CATSVRGTQPQHFF

SEQ ID NO: 23 - protein, 15 aa, Artificial Sequence
  Description of Artificial Sequence Synthetic peptide
CALSERGSGGFKTIF

SEQ ID NO: 24 - protein, 22 aa, Artificial Sequence
  Description of Artificial Sequence Synthetic peptide
CASSSGEKGQGAPVSSYEQYFF

SEQ ID NO: 25 - protein, 14 aa, Artificial Sequence
  Description of Artificial Sequence Synthetic peptide
CAFTYGGSQGNLIF

SEQ ID NO: 26 - protein, 16 aa, Artificial Sequence
  Description of Artificial Sequence Synthetic peptide
CASRASVGVGTGELFF

SEQ ID NO: 27 - protein, 10 aa, Artificial Sequence
  Description of Artificial Sequence Synthetic peptide
CALSGNMLTF

SEQ ID NO: 28 - protein, 15 aa, Artificial Sequence
  Description of Artificial Sequence Synthetic peptide
CASSEGVGQRDEQFF

SEQ ID NO: 29 - protein, 15 aa, Artificial Sequence
  Description of Artificial Sequence Synthetic peptide
CAASWEGGGADGLTF

SEQ ID NO: 30 - protein, 18 aa, Artificial Sequence
  Description of Artificial Sequence Synthetic peptide
CASRGQGVYRSSYNEQFF

SEQ ID NO: 31 - protein, 14 aa, Artificial Sequence
  Description of Artificial Sequence Synthetic peptide
CAVRDTGYGQNFVF

SEQ ID NO: 32 - protein, 15 aa, Artificial Sequence
  Description of Artificial Sequence Synthetic peptide
CASSPQGRINSPLHF

SEQ ID NO: 33 - protein, 12 aa, Artificial Sequence
  Description of Artificial Sequence Synthetic peptide
CILSAHDYKLSF

SEQ ID NO: 34 - protein, 14 aa, Artificial Sequence
  Description of Artificial Sequence Synthetic peptide
CASSQGGLNSPLHF

SEQ ID NO: 35 - protein, 15 aa, Artificial Sequence
  Description of Artificial Sequence Synthetic peptide
CAMRVAEGSQGNLIF

SEQ ID NO: 36 - protein, 15 aa, Artificial Sequence
  Description of Artificial Sequence Synthetic peptide
CASSPWGRGGSPLHF

SEQ ID NO: 37 - protein, 15 aa, Artificial Sequence
  Description of Artificial Sequence Synthetic peptide
CAVKFNTDQGGKLIF

SEQ ID NO: 38 - protein, 15 aa, Artificial Sequence
  Description of Artificial Sequence Synthetic peptide
CASSPQGRINSPLHF

SEQ ID NO: 39 - protein, 12 aa, Artificial Sequence
  Description of Artificial Sequence Synthetic peptide
CALGSSGTYKYI

SEQ ID NO: 40 - protein, 16 aa, Artificial Sequence
  Description of Artificial Sequence Synthetic peptide
CASSGPGQGHNQPQHF

SEQ ID NO: 41 - protein, 11 aa, Artificial Sequence
  Description of Artificial Sequence Synthetic peptide
CAMREANDMRF

SEQ ID NO: 42 - protein, 17 aa, Artificial Sequence
  Description of Artificial Sequence Synthetic peptide
CASSFLVLAVSYNEQFF

SEQ ID NO: 43 - protein, 15 aa, Artificial Sequence
  Description of Artificial Sequence Synthetic peptide
CAETLGLDQGGKLIF

SEQ ID NO: 44 - protein, 12 aa, Artificial Sequence
  Description of Artificial Sequence Synthetic peptide
CSTAGETDTQYF

SEQ ID NO: 45 - protein, 13 aa, Artificial Sequence
  Description of Artificial Sequence Synthetic peptide
CATDEGTASKLTF

SEQ ID NO: 46 - protein, 15 aa, Artificial Sequence
  Description of Artificial Sequence Synthetic peptide
CSVEWTSGSGETQYF

SEQ ID NO: 47 - protein, 13 aa, Artificial Sequence
  Description of Artificial Sequence Synthetic peptide
CATDAGSDYKLSF

SEQ ID NO: 48 - protein, 10 aa, Artificial Sequence
  Description of Artificial Sequence Synthetic peptide
CASSLEPQYF

SEQ ID NO: 49 - protein, 13 aa, Artificial Sequence
  Description of Artificial Sequence Synthetic peptide
CATDEGTGNQFYF

SEQ ID NO: 50 - protein, 14 aa, Artificial Sequence
  Description of Artificial Sequence Synthetic peptide
CASSWDTGTETQYF

SEQ ID NO: 51 - protein, 13 aa, Artificial Sequence
  Description of Artificial Sequence Synthetic peptide
CAFPSSGTYKYIF

SEQ ID NO: 52 - protein, 12 aa, Artificial Sequence
  Description of Artificial Sequence Synthetic peptide
CASTAGTDTQYF

SEQ ID NO: 53 - DNA, 9395 bp, Artificial Sequence
  Description of Artificial Sequence Synthetic polynucleotide
aagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatgcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgatatcctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctctagataatacgactcactatagggcccgggttggatccgccgccaccatgggcacaagcctgctgtgttgggtcgtgctgggctttctgggcacagatcatacaggcgccggtgtcagccagtctcctcggtacaaagtgaccaagcgcggacaggatgtggccctgagatgcgatcctatctctggccacgtgtccctgtactggtacagacaggctctcggacagggccccgagttcctgacctacttcaattacgaggcccagcaggacaagagcggcctgcctaacgatagattcagcgccgaaagacccgagggcagcatcagcacactgaccatccagagaaccgagcagagggacagcgccatgtacagatgtgccagctctcctgaactggccggacctcaagagacacagtacttcggccctggcaccagactgctggtgctggaagatctgaacaaggtgttccctccagaggtggccgtgttcgagccttctaaggccgagattgcccacacacagaaagccacactcgtgtgcctggccaccggcttttttcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggaattaccagcgccagctaccatcagggcgtgctgtctgccacaatcctgtacgagattctgctgggcaaagccactctgtacgccgtgctggtgtctgccctggtgctgatggccatggtcaagcggaaggacttcagagccaagagatccggctccggcgcaccggtgaaacagactttgaattttgaccttctcaagttggcaggagacgttgagtccaaccctgggcccatgctgacagccagcctgctgagagccgtgatcgcctctatctgtgtggtgtccagcatggctcagaaagtgacacaggcccagaccgagatcagcgtggtggaaaaagaagatgtgaccctggactgcgtgtacgagacacgggacaccacctactacctgttttggtacaagcagcctcctagcggcgagctggtgttcctgatcagacggaacagcttcgacgagcagaacgagatctccggccggtacagctggaacttccagaagtccaccagcagcttcaacttcaccatcaccgcctctcaggtggtggactccgccgtgtatttttgcgccctgtctgaaggcggcggaagccagggcaatctgatctttggcaagggcaccaagctgagcgtgaagcccaacattcagaaccccgatcctgccgtgtaccagctgagagacagcaagagcagcgacaagtccgtgtgtctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttccccagctccgatgtgccctgcgacgtgaagctggtcgagaagtcctttgagacagacaccaacctgaatttccagaacctgtccgtgatcggcttcagaatcctgctgctgaaagtggccggcttcaacctgctgatgaccctgagactctggtccagctaatgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgc

SEQ ID NO: 54 - DNA, 9386 bp, Artificial Sequence
  Description of Artificial Sequence Synthetic polynucleotide
aagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatgcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgatatcctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctctagataatacgactcactatagggcccgggttggatccgccgccaccatgctgctgctcctgctgcttctcggccctggaatttctctgctgctgcctggatctctggccggctctggacttggagctgtggtgtctcagcacccctcttgggtcatctgcaagagcggcaccagcgtgaagatcgagtgcagaagcctggacttccaggccaccaccatgttctggtacagacagttccccaagcagagcctgatgctgatggccacctctaacgagggcagcaaggccacatatgagcagggcgtcgagaaggacaagttcctgatcaaccacgccagcctgacactgagcaccctgacagtgacaagcgcccatcctgaggacagcagcttctacatctgcagcgccagagagggctacagaagctactttggccctggcaccagactgaccgtgctggaagatctgaagaacgtgttcccacctgaggtggccgtgttcgagccttctaaggccgagattgcccacacacagaaagccacactcgtgtgcctggccaccggcttttttcccgatcacgtcgaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggaattaccagcgccagctaccatcagggcgtgctgtctgccacaatcctgtacgagatcctgctgggcaaagccactctgtacgccgtgctggtgtctgccctggtcctgatggctatggtcaagagaaaggacagccggggccgggccaagcggtccggctccggcgcaccggtgaaacagactttgaattttgaccttctcaagttggcaggagacgttgagtccaaccctgggcccatgagactggtggccagagtgaccgtgttcctgaccttcggcaccatcatcgacgccaagacaacccagcctcctagcatggattgtgccgagggcagagccgccaacctgccttgtaatcacagcaccatcagcggcaacgagtacgtgtactggtacagacagatccactctcagggccctcagtacatcatccacggcctgaagaacaacgagacaaacgagatggccagcctgatcatcaccgaggacagaaagagcagcaccctgatcctgcctcacgccacactgagagataccgccgtgtactactgcatcgtgcgggacagaagctacggccagaacttcgtgtttggccctggcaccagactgagcgtgctgccctatattcagaaccccgatccagccgtgtaccagctgagagacagcaagtccagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttccccagctccgatgtgccctgcgacgtgaagctggtggaaaagagcttcgagacagacaccaacctgaacttccagaacctgagcgtgatcggcttccgcatcctgctgctgaaagtggccggcttcaacctgctgatgaccctgagactgtggtccagctaatgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgc

SEQ ID NO: 55 - DNA, 9398 bp, Artificial Sequence
  Description of Artificial Sequence Synthetic polynucleotide
aagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatgcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgatatcctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctctagataatacgactcactatagggcccgggttggatccgccgccaccatggatacctggctcgtgtgctgggccatcttcagcctgctgaaagccggactgaccgagcctgaagtgacccagacacctagccaccaagtgacacagatgggccaagaagtgatcctgcgctgcgtgcccatcagcaaccacctgtacttctactggtacagacagatcctgggccagaaagtggagttcctggtgtccttctacaacaacgagatcagcgagaagtccgagatcttcgacgaccagttcagcgtggaaagacccgacggcagcaacttcaccctgaagatcagaagcaccaagctggaggacagcgccatgtacttttgcgccagcagagcctctgtcggcgttggaacaggcgagctgtttttcggcgagggcagcagactgaccgtgctggaagatctgaagaacgtgttcccacctgaggtggccgtgttcgagccttctaaggccgagattgcccacacacagaaagccacactcgtgtgcctggccaccggcttttttcccgatcacgtggaactgtcttggtgggtcaacggcaaagaggtgcacagcggcgtctgtaccgatcctcagccactgaaagagcagcccgctctgaacgacagcagatactgcctgtcctccagactgagagtgtccgccaccttctggcagaaccccagaaaccacttcagatgccaggtgcagttctacggcctgagcgagaacgatgagtggacccaggatagagccaagcctgtgactcagatcgtgtctgccgaagcctggggcagagccgattgtggaattaccagcgccagctaccatcagggcgtgctgtctgccacaatcctgtacgagatcctgctgggcaaagccactctgtacgccgtgctggtgtctgccctggtgctgatggccatggtcaagcggaaggatagcagaggcagagccaagagatccggctccggcgcaccggtgaaacagactttgaattttgaccttctcaagttggcaggagacgttgagtccaaccctgggcccatgaccagagtgtctctgctgtgggccgtcgtggtgtccacatgtctggaatctggcatggcccagaccgtgacacagagccagcctgagatgtctgtgcaagaggccgagacagtgaccctgagctgcacctacgataccagcgagagcaactactacctgttctggtacaagcagcctcctagccggcagatgatcctggtcatcagacaagaggcctataagcagcagaacgccaccgagaacagattcagcgtgaacttccagaaggccgccaagagcttcagcctgaagatcagcgatagccagctgggcgacaccgccatgtacttttgcgcctttacctacggcggcagccagggcaatctgatctttggcaagggcaccaagctgagcgtgaagcccaacattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttccccagctccgatgtgccctgcgacgtgaagctggtggaaaagagcttcgagacagacaccaacctgaatttccagaacctgtccgtgatcggcttcagaatcctgctgctgaaagtggccggcttcaacctgctgatgaccctgcgactttggagcagctaatgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgc

SEQ ID NO: 56 - DNA, 9365 bp, Artificial Sequence
  Description of Artificial Sequence Synthetic polynucleotide
aagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatgcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgatatcctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctctagataatacgactcactatagggcccgggttggatccgccgccaccatgggacctcagctgctgggatacgtggtgctgtgtctgcttggagccggacctctggaagcccaagtgacacagaaccccagatacctgatcaccgtgaccggcaagaaactgaccgtgacctgcagccagaacatgaaccacgagtacatgagctggtacagacaggaccctggcctgggcctgagacagatctactacagcatgaacgtggaagtgaccgacaagggcgacgtgcccgagggctacaaggtgtccagaaaagagaagcggaacttcccactgatcctggaaagcccatctcctaaccagaccagcctgtacttctgcgccagctctcctcagggcagaatcaacagccctctgcacttcggcaacggcaccagactgacagtgaccgaggacctgaacaaggtgttccctccagaggtggccgtgttcgagccttctaaggccgagattgcccacacacagaaagccacactcgtgtgcctggccaccggcttttttcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccctcggaaccacttcagatgccaggtgcagttctacggcctgagcgagaacgatgagtggacccaggatagagccaagcctgtgactcagatcgtgtctgccgaagcctggggcagagccgattgtggaattaccagcgccagctaccatcagggcgtgctgtctgccacaatcctgtacgagatcctgctgggcaaagccactctgtacgccgtgctggtttctgctctggtgctgatggccatggtcaagcggaaggacttcagagccaagagatccggctccggcgcaccggtgaaacagactttgaattttgaccttctcaagttggcaggagacgttgagtccaaccctgggcccatgtggggagtgttcctgctgtacgtgtccatgaagatgggcggcaccaccggccagaacatcgatcagcctacagagatgaccgccaccgagggcgccatcgtgcagatcaattgcacctaccagaccagcggcttcaacggcctgttctggtatcagcagcatgccggcgaggcccctaccttcctgagctacaatgtgctggacggcctggaagagaagggcagattcagcagcttcctgtccagaagcaagggctacagctacctgctgctgaaagaactgcagatgaaggacagcgcctcctacctgtgcgccgtcagagatacaggctacggccagaatttcgtgttcggccctggcaccagactgagcgtgctgccctatattcagaaccccgatcctgccgtgtaccagctgagagacagcaagagcagcgacaagagcgtgtgcctgttcaccgacttcgacagccagaccaacgtgtcccagagcaaggactccgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttcccaagcagcgacgtgccctgcgacgtgaagctggtggaaaagagcttcgagacagacaccaacctgaacttccagaacctgagcgtgatcggcttcagaatcctgctgcttaaggtggccggcttcaatctgctgatgaccctgagactgtggtccagctaatgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgc

SEQ ID NO: 57 - DNA, 9386 bp, Artificial Sequence
  Description of Artificial Sequence Synthetic polynucleotide
aagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatgcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgatatcctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctctagataatacgactcactatagggcccgggttggatccgccgccaccatgaccatccggctgctgtgctatatgggcttctacttcctcggagccggcctgatggaagccgacatctaccagacacctagatacctggtcatcggcaccggcaagaaaatcaccctggaatgcagccagaccatgggccacgacaagatgtactggtatcagcaggaccccggcatggaactgcacctgatccactacagctacggcgtgaacagcaccgagaagggcgatctgtctagcgagagcaccgtgtccagaatccggaccgagcacttcccactgacactggaaagcgctagacccagccacaccagccagtacctgtgtgcctcttctggacctggacagggccacaatcagcctcagcactttggcgacggcaccagactgagcatcctggaagatctgaacaaggtgttccctccagaggtggccgtgttcgagccttctaaggccgagattgcccacacacagaaagccacactcgtgtgcctggccaccggcttttttcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggttcagttctacggcctgagcgagaacgacgagtggacccaggatagagccaagcctgtgacacagatcgtgtctgccgaagcctggggcagagccgattgtggaattaccagcgccagctaccatcagggcgtgctgtctgccacaatcctgtacgagatcctgctgggcaaagccactctgtacgccgtgctggtgtctgccctggtgctgatggccatggtcaagcggaaggacttcagagccaagagatccggctccggcgcaccggtgaaacagactttgaattttgaccttctcaagttggcaggagacgttgagtccaaccctgggcccatgctgacagcctctctgctgagagccgtgatcgccagcatctgtgtggtgtctagcatggcccagaaagtgacacaggcccagaccgagatcagcgtggtggaaaaagaagatgtgaccctggactgcgtgtacgagacacgggacaccacctactacctgttctggtacaagcagcctcctagcggcgagctggtgttcctgatcagacggaacagcttcgacgagcagaacgagatctccggccggtacagctggaacttccagaagtccaccagcagcttcaacttcaccatcaccgccagccaggtggtggatagcgccgtgtatttttgtgccctgggcagcagcggcacctacaagtacatctttggcaccggcaccagactgaaggtgctggccaacattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttccccagctccgatgtgccctgcgacgtgaagctggtcgagaagtccttcgagacagacaccaacctgaatttccagaacctgagcgtgatcggcttcagaatcctgctgctgaaagtggccggcttcaacctgctgatgaccctgagactctggtccagctaatgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgc

SEQ ID NO: 58 - DNA, 9407 bp, Artificial Sequence
  Description of Artificial Sequence Synthetic polynucleotide
aagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatgcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgatatcctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctctagataatacgactcactatagggcccgggttggatccgccgccaccatgctgctgctcctgctgcttctcggccctggaatttctctgctgctgcctggatctctggccggctctggacttggagctgtggtgtctcagcacccctcttgggtcatctgcaagagcggcaccagcgtgaagatcgagtgcagaagcctggacttccaggccaccaccatgttctggtacagacagttccccaagcagagcctgatgctgatggccacctctaacgagggctgcaaggccacatatgagcagggcgtcgagaaggacaagttcctgatcaaccacgccagcctgacactgagcaccctgacagtgacaagcgcccatcctgaggacagcagcttctacatctgtagcacagccggcgagacagacacccagtattttggccctggcaccagactgaccgtgctggaagatctgaagaacgtgttcccacctgaggtggccgtgttcgagccttctaaggccgagattgcccacacacagaaagccacactcgtgtgtctggccaccggcttctatcccgatcacgtcgaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggaattaccagcgccagctaccatcagggcgtgctgtctgccacaatcctgtacgagatcctgctgggcaaagccactctgtacgccgtgctggtgtctgccctggtcctgatggctatggtcaagagaaaggacagccggggcagagccaagagatccggctccggcgcaccggtgaaacagactttgaattttgaccttctcaagttggcaggagacgttgagtccaaccctgggcccatggccggaatcagagccctgttcatgtacctgtggctgcagctggactgggtgtccagaggcgaatctgtgggactgcatctgcccacactgagcgtgcaagagggcgacaacagcatcatcaactgcgcctacagcaacagcgccagcgactacttcatctggtacaagcaagagagcggcaagggccctcagttcatcatcgacatccggtccaacatggacaagcggcaaggccagagagtgaccgtcctgctgaacaagaccgtgaagcacctgagcctgcagatcgccgctacacagcctggcgatagcgccgtgtacttctgtgccgaaacactgggcctcgaccaaggcggcaagctgatctttggacagggcacagagctgagcgtgaagcccaacattcagaaccccgatcctgccgtgtaccagctgagagacagcaagtccagcgacaagagcgtgtgcctgttcaccgacttcgacagccagaccaacgtgtcccagagcaaggacagcgacgtgtacatcaccgataagtgcgtgctggacatgcggagcatggacttcaagagcaactccgccgtggcctggtccaacaagagcgatttcgcctgcgccaacgccttcaacaactctattatccccgaggacacattcttccccagctccgatgtgccctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctaatgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgc

SEQ ID NO: 59 - protein, 295 aa, Artificial Sequence
  Description of Artificial Sequence Synthetic polypeptide
EPEVTQTPSHQVTQMGQEVILRCVPISNHLYFYWYRQILGQKVEFLVSFYNNEISEKSEIFDDQFSVERPDGSNFTLKIRSTKLEDSAMYFCASRASVGVGTGELFFGEGSRLTVLEDLKNVFPPEVAVFEPSKAEIAHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 60 - protein, 256 aa, Artificial Sequence
  Description of Artificial Sequence Synthetic polypeptide
AQTVTQSQPEMSVQEAETVTLSCTYDTSESNYYLFWYKQPPSRQMILVIRQEAYKQQNATENRFSVNFQKAAKSFSLKISDSQLGDTAMYFCAFTYGGSQGNLIFGKGTKLSVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 61 - protein, 291 aa, Artificial Sequence
  Description of Artificial Sequence Synthetic polypeptide
GVSQSPRYKVTKRGQDVALRCDPISGHVSLYWYRQALGQGPEFLTYFNYEAQQDKSGLPNDRFSAERPEGSISTLTIQRTEQRDSAMYRCASSPELAGPQETQYFGPGTRLLVLEDLNKVFPPEVAVFEPSKAEIAHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 62 - protein, 257 aa, Artificial Sequence
  Description of Artificial Sequence Synthetic polypeptide
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEGGGSQGNLIFGKGTKLSVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 63 - protein, 311 aa, Artificial Sequence
  Description of Artificial Sequence Synthetic polypeptide
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSGPGQGHNQPQHFGDGTRLSILEDLNKVFPPEVAVFEPSKAEIAHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 64 - protein, 254 aa, Artificial Sequence
  Description of Artificial Sequence Synthetic polypeptide
QKVTQAQTEISVVEKEDVTLDCVYETRDTTYYLFWYKQPPSGELVFLIRRNSFDEQNEISGRYSWNFQKSTSSFNFTITASQVVDSAVYFCALGSSGTYKYIFGTGTRLKVLANIQNPDPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 65 - protein, 289 aa, Artificial Sequence
  Description of Artificial Sequence Synthetic polypeptide
QVTQNPRYLITVTGKKLTVTCSQNMNHEYMSWYRQDPGLGLRQIYYSMNVEVTDKGDVPEGYKVSRKEKRNFPLILESPSPNQTSLYFCASSPQGRINSPLHFGNGTRLTVTEDLNKVFPPEVAVFEPSKAEIAHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 66 - protein, 251 aa, Artificial Sequence
  Description of Artificial Sequence Synthetic polypeptide
QNIDQPTEMTATEGAIVQINCTYQTSGFNGLFWYQQHAGEAPTFLSYNVLDGLEEKGRFSSFLSRSKGYSYLLLKELQMKDSASYLCAVRDTGYGQNFVFGPGTRLSVLPYIQNPDPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 67 - protein, 296 aa, Artificial Sequence
  Description of Artificial Sequence Synthetic polypeptide
GSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSAREGYRSYFGPGTRLTVLEDLKNVFPPEVAVFEPSKAEIAHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 68 - protein, 250 aa, Artificial Sequence
  Description of Artificial Sequence Synthetic polypeptide
KTTQPPSMDCAEGRAANLPCNHSTISGNEYVYWYRQIHSQGPQYIIHGLKNNETNEMASLIITEDRKSSTLILPHATLRDTAVYYCIVRDRSYGQNFVFGPGTRLSVLPYIQNPDPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 69 - protein, 297 aa, Artificial Sequence
  Description of Artificial Sequence Synthetic polypeptide
GSGLGAVVSQHPSWVICKSGTSVKIECRSLDFQATTMFWYRQFPKQSLMLMATSNEGCKATYEQGVEKDKFLINHASLTLSTLTVTSAHPEDSSFYICSTAGETDTQYFGPGTRLTVLEDLKNVFPPEVAVFEPSKAEIAHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 70 - protein, 259 aa, Artificial Sequence
  Description of Artificial Sequence Synthetic polypeptide
ESVGLHLPTLSVQEGDNSIINCAYSNSASDYFIWYKQESGKGPQFIIDIRSNMDKRQGQRVTVLLNKTVKHLSLQIAATQPGDSAVYFCAETLGLDQGGKLIFGQGTELSVKPNIQNPDSVKPNPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 71 - DNA, 20 bp, Artificial Sequence
  Description of Artificial Sequence Synthetic primer
aactgcacgtaccagacatc

SEQ ID NO: 72 - DNA, 18 bp, Artificial Sequence
  Description of Artificial Sequence Synthetic primer
gatgtgcaccaagactcc

SEQ ID NO: 73 - DNA, 19 bp, Artificial Sequence
  Description of Artificial Sequence Synthetic primer
aagatcaggtcaacgttgc

SEQ ID NO: 74 - DNA, 21 bp, Artificial Sequence
  Description of Artificial Sequence Synthetic primer
ctccatggactcatatgaagg

SEQ ID NO: 75 - DNA, 19 bp, Artificial Sequence
  Description of Artificial Sequence Synthetic primer
cttttcctgagtgtccgag

SEQ ID NO: 76 - DNA, 21 bp, Artificial Sequence
  Description of Artificial Sequence Synthetic primer
caccctgacctgcaactatac

SEQ ID NO: 77 - DNA, 20 bp, Artificial Sequence
  Description of Artificial Sequence Synthetic primer
agctgcacgtactctgtcag

SEQ ID NO: 78 - DNA, 18 bp, Artificial Sequence
  Description of Artificial Sequence Synthetic primer
ctcactggagttgggatg

SEQ ID NO: 79 - DNA, 17 bp, Artificial Sequence
  Description of Artificial Sequence Synthetic primer
gccaccctggttaaagg

SEQ ID NO: 80 - DNA, 19 bp, Artificial Sequence
  Description of Artificial Sequence Synthetic primer
cactgtctctgaaggagcc

SEQ ID NO: 81 - DNA, 20 bp, Artificial Sequence
  Description of Artificial Sequence Synthetic primer
gagctgaggtgcaactactc

SEQ ID NO: 82 - DNA, 18 bp, Artificial Sequence
  Description of Artificial Sequence Synthetic primer
ctaacagaggccacccag

SEQ ID NO: 83 - DNA, 20 bp, Artificial Sequence
  Description of Artificial Sequence Synthetic primer
tggtatgtccaatatcctgg

SEQ ID NO: 84 - DNA, 19 bp, Artificial Sequence
  Description of Artificial Sequence Synthetic primer
caagtggagcagagtcctc

SEQ ID NO: 85 - DNA, 18 bp, Artificial Sequence
  Description of Artificial Sequence Synthetic primer
cartgttccagagggagc

SEQ ID NO: 86 - DNA, 19 bp, Artificial Sequence
  Description of Artificial Sequence Synthetic primer
catccttcaaccctgagtg

SEQ ID NO: 87 - DNA, 19 bp, Artificial Sequence
  Description of Artificial Sequence Synthetic primer
cagcgcctcagactacttc

SEQ ID NO: 88 - DNA, 22 bp, Artificial Sequence
  Description of Artificial Sequence Synthetic primer
aagataactcaaacccaaccag

SEQ ID NO: 89 - DNA, 19 bp, Artificial Sequence
  Description of Artificial Sequence Synthetic primer
agtggagctgaagtgcaac

SEQ ID NO: 90 - DNA, 19 bp, Artificial Sequence
  Description of Artificial Sequence Synthetic primer
ggagaagaggatcctcagg

SEQ ID NO: 91 - DNA, 22 bp, Artificial Sequence
  Description of Artificial Sequence Synthetic primer
tccagtatctaaacaaagagcc

SEQ ID NO: 92 - DNA, 19 bp, Artificial Sequence
  Description of Artificial Sequence Synthetic primer
aggtaactcaagcgcagac

SEQ ID NO: 93 - DNA, 20 bp, Artificial Sequence
  Description of Artificial Sequence Synthetic primer
cacagtcagcggtttaagag

SEQ ID NO: 94 - DNA, 18 bp, Artificial Sequence
  Description of Artificial Sequence Synthetic primer
ttcctgcagctctgagtg

SEQ ID NO: 95 - DNA, 20 bp, Artificial Sequence
  Description of Artificial Sequence Synthetic primer
gtcctccagacctgattctc

SEQ ID NO: 96 - DNA, 19 bp, Artificial Sequence
  Description of Artificial Sequence Synthetic primer
tgcttatgagaacactgcg

SEQ ID NO: 97 - DNA, 20 bp, Artificial Sequence
  Description of Artificial Sequence Synthetic primer
ctcagtcactgcatgttcag

SEQ ID NO: 98 - DNA, 19 bp, Artificial Sequence
  Description of Artificial Sequence Synthetic primer
ggacttcaccacgtactgc

SEQ ID NO: 99 - DNA, 19 bp, Artificial Sequence
  Description of Artificial Sequence Synthetic primer
gcaaacctgccttgtaatc

SEQ ID NO: 100 - DNA, 19 bp, Artificial Sequence
  Description of Artificial Sequence Synthetic primer
agccaaattcaatggagag

SEQ ID NO: 101 - DNA, 22 bp, Artificial Sequence
  Description of Artificial Sequence Synthetic primer
tcagtttctaagcatccaagag

SEQ ID NO: 102 - DNA, 21 bp, Artificial Sequence
  Description of Artificial Sequence Synthetic primer
gcaagttaagcaaaattcacc

SEQ ID NO: 103 - DNA, 19 bp, Artificial Sequence
  Description of Artificial Sequence Synthetic primer
caacaaccagtgcagagtc

SEQ ID NO: 104 - DNA, 20 bp, Artificial Sequence
  Description of Artificial Sequence Synthetic primer
agaactggagcagagtcctc

SEQ ID NO: 105 - DNA, 20 bp, Artificial Sequence
  Description of Artificial Sequence Synthetic primer
ggtcaacagctgaatcagag

SEQ ID NO: 106 - DNA, 21 bp, Artificial Sequence
  Description of Artificial Sequence Synthetic primer
gaagacaaggtggtacaaagc

SEQ ID NO: 107 - DNA, 20 bp, Artificial Sequence
  Description of Artificial Sequence Synthetic primer
gcacatatgacaccagtgag

SEQ ID NO: 108 - DNA, 18 bp, Artificial Sequence
  Description of Artificial Sequence Synthetic primer
ctgttcctgagcatgcag

SEQ ID NO: 109 - DNA, 21 bp, Artificial Sequence
  Description of Artificial Sequence Synthetic primer
gcatctgtgactatgaactgc

SEQ ID NO: 110 - DNA, 20 bp, Artificial Sequence
  Description of Artificial Sequence Synthetic primer
aatgaagtggagcagagtcc

SEQ ID NO: 111 - DNA, 19 bp, Artificial Sequence
  Description of Artificial Sequence Synthetic primer
gaccagcttgacatcacag

SEQ ID NO: 112 - DNA, 21 bp, Artificial Sequence
  Description of Artificial Sequence Synthetic primer
tcgatgatcaattctcagttg

SEQ ID NO: 113 - DNA, 20 bp, Artificial Sequence
  Description of Artificial Sequence Synthetic primer
caaaatacctggtcacacag

SEQ ID NO: 114 - DNA, 18 bp, Artificial Sequence
  Description of Artificial Sequence Synthetic primer
tcgcttctcacctgaatg

SEQ ID NO: 115 - DNA, 20 bp, Artificial Sequence
  Description of Artificial Sequence Synthetic primer
gattctcaggkckccagttc

SEQ ID NO: 116 - DNA, 19 bp, Artificial Sequence
  Description of Artificial Sequence Synthetic primer
gtaccaacaggycctgggt

SEQ ID NO: 117 - DNA, 19 bp, Artificial Sequence
  Description of Artificial Sequence Synthetic primer
actcagaccccaaaattcc

SEQ ID NO: 118 - DNA, 19 bp, Artificial Sequence
  Description of Artificial Sequence Synthetic primer
actggcaaaggagaagtcc

SEQ ID NO: 119 - DNA, 20 bp, Artificial Sequence
  Description of Artificial Sequence Synthetic primer
trtgatccaatttcaggtca

SEQ ID NO: 120 - DNA, 19 bp, Artificial Sequence
  Description of Artificial Sequence Synthetic primer
gswtctytgcagaraggcc

SEQ ID NO: 121 - DNA, 19 bp, Artificial Sequence
  Description of Artificial Sequence Synthetic primer
gatcacagcaactggacag

SEQ ID NO: 122 - DNA, 21 bp, Artificial Sequence
  Description of Artificial Sequence Synthetic primer
tgtwctggtatcgacaagacc

SEQ ID NO: 123 - DNA, 19 bp, Artificial Sequence
  Description of Artificial Sequence Synthetic primer
cgattttctgcagagacgc

SEQ ID NO: 124 - DNA, 20 bp, Artificial Sequence
  Description of Artificial Sequence Synthetic primer
argtgacagaratgggacaa

SEQ ID NO: 125 - DNA, 19 bp, Artificial Sequence
  Description of Artificial Sequence Synthetic primer
agcgataaaggaagcatcc

SEQ ID NO: 126 - DNA, 21 bp, Artificial Sequence
  Description of Artificial Sequence Synthetic primer
ccaacaatcgattcttagctg

SEQ ID NO: 127 - DNA, 20 bp, Artificial Sequence
  Description of Artificial Sequence Synthetic primer
agtgaccctgagttgttctc

SEQ ID NO: 128 - DNA, 22 bp, Artificial Sequence
  Description of Artificial Sequence Synthetic primer
gtctttgatgaaacaggtatgc

SEQ ID NO: 129 - DNA, 18 bp, Artificial Sequence
  Description of Artificial Sequence Synthetic primer
cagacccccagacacaag

SEQ ID NO: 130 - DNA, 21 bp, Artificial Sequence
  Description of Artificial Sequence Synthetic primer
catagatgagtcaggaatgcc

SEQ ID NO: 131 - DNA, 22 bp, Artificial Sequence
  Description of Artificial Sequence Synthetic primer
agttgtgaacagaatttgaacc

SEQ ID NO: 132 - DNA, 20 bp, Artificial Sequence
  Description of Artificial Sequence Synthetic primer
aagtttctcatcaaccatgc

SEQ ID NO: 133 - DNA, 19 bp, Artificial Sequence
  Description of Artificial Sequence Synthetic primer
gcgattctcatctcaatgc

SEQ ID NO: 134 - DNA, 22 bp, Artificial Sequence
  Description of Artificial Sequence Synthetic primer
cctacggttgatctattactcc

SEQ ID NO: 135 - DNA, 22 bp, Artificial Sequence
  Description of Artificial Sequence Synthetic primer
actacacctcatccactattcc

SEQ ID NO: 136 - DNA, 19 bp, Artificial Sequence
  Description of Artificial Sequence Synthetic primer
tggtatcgacaagacccag

SEQ ID NO: 137 - DNA, 19 bp, Artificial Sequence
  Description of Artificial Sequence Synthetic primer
ttctggtaccgtcagcaac

SEQ ID NO: 138 - DNA, 20 bp, Artificial Sequence
  Description of Artificial Sequence Synthetic primer
tccagctgctcttctactcc

SEQ ID NO: 139 - DNA, 20 bp, Artificial Sequence
  Description of Artificial Sequence Synthetic primer
tagaactggacttgacagcg

SEQ ID NO: 140 - DNA, 21 bp, Artificial Sequence
  Description of Artificial Sequence Synthetic primer
gcacccacatttctktcttac

SEQ ID NO: 141 - DNA, 21 bp, Artificial Sequence
  Description of Artificial Sequence Synthetic primer
cactctgtgtccaatgcttac

SEQ ID NO: 142 - DNA, 21 bp, Artificial Sequence
  Description of Artificial Sequence Synthetic primer
atgcacctattcagtctctgg

SEQ ID NO: 143 - DNA, 23 bp, Artificial Sequence
  Description of Artificial Sequence Synthetic primer
attatatcacgtggtaccaacag

SEQ ID NO: 144 - DNA, 20 bp, Artificial Sequence
  Description of Artificial Sequence Synthetic primer
tacacagacagctcctccac

SEQ ID NO: 145 - DNA, 19 bp, Artificial Sequence
  Description of Artificial Sequence Synthetic primer
tggtaccgacaagatccag

SEQ ID NO: 146 - DNA, 20 bp, Artificial Sequence
  Description of Artificial Sequence Synthetic primer
acaatttgcagtggtacagg

SEQ ID NO: 147 - DNA, 20 bp, Artificial Sequence
  Description of Artificial Sequence Synthetic primer
gtcaacaccttcagcttctc

SEQ ID NO: 148 - DNA, 20 bp, Artificial Sequence
  Description of Artificial Sequence Synthetic primer
agagtgaaacctccttccac

SEQ ID NO: 149 - DNA, 21 bp, Artificial Sequence
  Description of Artificial Sequence Synthetic primer
tttgaggctgaatttaagagg

SEQ ID NO: 150 - DNA, 19 bp, Artificial Sequence
  Description of Artificial Sequence Synthetic primer
aaccaaggactccagcttc

SEQ ID NO: 151 - DNA, 19 bp, Artificial Sequence
  Description of Artificial Sequence Synthetic primer
atcagaggttttgaggctg

SEQ ID NO: 152 - DNA, 21 bp, Artificial Sequence
  Description of Artificial Sequence Synthetic primer
gaaaccacttctttccacttg

SEQ ID NO: 153 - DNA, 22 bp, Artificial Sequence
  Description of Artificial Sequence Synthetic primer
gaaagaactgcactcttcaatg

SEQ ID NO: 154 - DNA, 21 bp, Artificial Sequence
  Description of Artificial Sequence Synthetic primer
aagatggaaggtttacagcac

SEQ ID NO: 155 - DNA, 21 bp, Artificial Sequence
  Description of Artificial Sequence Synthetic primer
tcagacagtgcctcaaactac

SEQ ID NO: 156 - DNA, 21 bp, Artificial Sequence
  Description of Artificial Sequence Synthetic primer
cagtgaaacatctctctctgc

SEQ ID NO: 157 - DNA, 19 bp, Artificial Sequence
  Description of Artificial Sequence Synthetic primer
aggctgtgactctggactg

SEQ ID NO: 158 - DNA, 20 bp, Artificial Sequence
  Description of Artificial Sequence Synthetic primer
gtccagtactccagacaacg

SEQ ID NO: 159 - DNA, 20 bp, Artificial Sequence
  Description of Artificial Sequence Synthetic primer
ccaccatgaactgcagttac

SEQ ID NO: 160 - DNA, 19 bp, Artificial Sequence
  Description of Artificial Sequence Synthetic primer
tgacagttccttccacctg

SEQ ID NO: 161 - DNA, 19 bp, Artificial Sequence
  Description of Artificial Sequence Synthetic primer
tgtgaccttggactgtgtg

SEQ ID NO: 162 - DNA, 21 bp, Artificial Sequence
  Description of Artificial Sequence Synthetic primer
tctggtataggcaagatcctg

SEQ ID NO: 163 - DNA, 20 bp, Artificial Sequence
  Description of Artificial Sequence Synthetic primer
aacttggttctcaactgcag

SEQ ID NO: 164 - DNA, 21 bp, Artificial Sequence
  Description of Artificial Sequence Synthetic primer
ctgactctgtgaacaatttgc

SEQ ID NO: 165 - DNA, 21 bp, Artificial Sequence
  Description of Artificial Sequence Synthetic primer
tgcattattgatagccatacg

SEQ ID NO: 166 - DNA, 21 bp, Artificial Sequence
  Description of Artificial Sequence Synthetic primer
tgccttacactggtacagatg

SEQ ID NO: 167 - DNA, 19 bp, Artificial Sequence
  Description of Artificial Sequence Synthetic primer
tataagcaaaggcctggtg

SEQ ID NO: 168 - DNA, 18 bp, Artificial Sequence
  Description of Artificial Sequence Synthetic primer
cgacagattcactcccag

SEQ ID NO: 169 - DNA, 20 bp, Artificial Sequence
  Description of Artificial Sequence Synthetic primer
ttcacttgccttgtaaccac

SEQ ID NO: 170 - DNA, 21 bp, Artificial Sequence
  Description of Artificial Sequence Synthetic primer
ctcactgtgtactgcaactcc

SEQ ID NO: 171 - DNA, 20 bp, Artificial Sequence
  Description of Artificial Sequence Synthetic primer
ctgctgaaggtcctacattc

SEQ ID NO: 172 - DNA, 18 bp, Artificial Sequence
  Description of Artificial Sequence Synthetic primer
agaagcatggtgaagcac

SEQ ID NO: 173 - DNA, 20 bp, Artificial Sequence
  Description of Artificial Sequence Synthetic primer
atctcaccataaactgcacg

SEQ ID NO: 174 - DNA, 19 bp, Artificial Sequence
  Description of Artificial Sequence Synthetic primer
acctggctatggtacaagc

SEQ ID NO: 175 - DNA, 19 bp, Artificial Sequence
  Description of Artificial Sequence Synthetic primer
atctctggttgtccacgag

SEQ ID NO: 176 - DNA, 19 bp, Artificial Sequence
  Description of Artificial Sequence Synthetic primer
cagcaggcagatgattctc

SEQ ID NO: 177 - DNA, 21 bp, Artificial Sequence
  Description of Artificial Sequence Synthetic primer
tcaaccacttcagacagactg

SEQ ID NO: 178 - DNA, 21 bp, Artificial Sequence
  Description of Artificial Sequence Synthetic primer
ggaggcggaaatattaaagac

SEQ ID NO: 179 - DNA, 20 bp, Artificial Sequence
  Description of Artificial Sequence Synthetic primer
ttgtttatgctgagctcagg

SEQ ID NO: 180 - DNA, 21 bp, Artificial Sequence
  Description of Artificial Sequence Synthetic primer
tgttgctcttgaagtccatag

SEQ ID NO: 181 - DNA, 20 bp, Artificial Sequence
  Description of Artificial Sequence Synthetic primer
ttcactctgaagatccggtc

SEQ ID NO: 182 - DNA, 21 bp, Artificial Sequence
  Description of Artificial Sequence Synthetic primer
aatcttcacatcaattccctg

SEQ ID NO: 183 - DNA, 18 bp, Artificial Sequence
  Description of Artificial Sequence Synthetic primer
cctgcagccagaagactc

SEQ ID NO: 184 - DNA, 18 bp, Artificial Sequence
  Description of Artificial Sequence Synthetic primer
cttggagctggrsgactc

SEQ ID NO: 185 - DNA, 19 bp, Artificial Sequence
  Description of Artificial Sequence Synthetic primer
tctgagctgaatgtgaacg

SEQ ID NO: 186 - DNA, 20 bp, Artificial Sequence
  Description of Artificial Sequence Synthetic primer
gtgtrcccaggatatgaacc

SEQ ID NO: 187 - DNA, 21 bp, Artificial Sequence
  Description of Artificial Sequence Synthetic primer
tggttatagtgtctccagagc

SEQ ID NO: 188 - DNA, 20 bp, Artificial Sequence
  Description of Artificial Sequence Synthetic primer
tcyactctgamgwtccagcg

SEQ ID NO: 189 - DNA, 17 bp, Artificial Sequence
  Description of Artificial Sequence Synthetic primer
tgrmgatycagcgcaca

SEQ ID NO: 190 - DNA, 19 bp, Artificial Sequence
  Description of Artificial Sequence Synthetic primer
gtaccaacagagcctggac

SEQ ID NO: 191 - DNA, 19 bp, Artificial Sequence
  Description of Artificial Sequence Synthetic primer
tccycctcactctggagtc

SEQ ID NO: 192 - DNA, 20 bp, Artificial Sequence
  Description of Artificial Sequence Synthetic primer
gactccactctcaagatcca

SEQ ID NO: 193 - DNA, 19 bp, Artificial Sequence
  Description of Artificial Sequence Synthetic primer
cyactctgargatccagcc

SEQ ID NO: 194 - DNA, 21 bp, Artificial Sequence
  Description of Artificial Sequence Synthetic primer
cattctgaactgaacatgagc

SEQ ID NO: 195 - DNA, 21 bp, Artificial Sequence
  Description of Artificial Sequence Synthetic primer
attctactctgaaggtgcagc

SEQ ID NO: 196 - DNA, 20 bp, Artificial Sequence
  Description of Artificial Sequence Synthetic primer
ataacttccaatccaggagg

SEQ ID NO: 197 - DNA, 22 bp, Artificial Sequence
  Description of Artificial Sequence Synthetic primer
gaaagattttcagctaagtgcc

SEQ ID NO: 198 - DNA, 19 bp, Artificial Sequence
  Description of Artificial Sequence Synthetic primer
tgttcactggtaccgacag

SEQ ID NO: 199 - DNA, 20 bp, Artificial Sequence
  Description of Artificial Sequence Synthetic primer
cgattttctgctgaatttcc

SEQ ID NO: 200 - DNA, 20 bp, Artificial Sequence
  Description of Artificial Sequence Synthetic primer
ttcctctcactgtgacatcg

SEQ ID NO: 201 - DNA, 20 bp, Artificial Sequence
  Description of Artificial Sequence Synthetic primer
actctgacagtgaccagtgc

SEQ ID NO: 202 - DNA, 19 bp, Artificial Sequence
  Description of Artificial Sequence Synthetic primer
gcaatcctgtcctcagaac

SEQ ID NO: 203 - DNA, 21 bp, Artificial Sequence
  Description of Artificial Sequence Synthetic primer
gatggatacagtgtctctcga

SEQ ID NO: 204 - DNA, 20 bp, Artificial Sequence
  Description of Artificial Sequence Synthetic primer
cagagaagggagatctttcc

SEQ ID NO: 205 - DNA, 20 bp, Artificial Sequence
  Description of Artificial Sequence Synthetic primer
ttcyccctgatyctggagtc

SEQ ID NO: 206 - DNA, 21 bp, Artificial Sequence
  Description of Artificial Sequence Synthetic primer
tctgactgtgagcaacatgag

SEQ ID NO: 207 - DNA, 21 bp, Artificial Sequence
  Description of Artificial Sequence Synthetic primer
agaatctctcagcctccagac

SEQ ID NO: 208 - DNA, 20 bp, Artificial Sequence
  Description of Artificial Sequence Synthetic primer
ttctgatggctcaaacacag

SEQ ID NO: 209 - protein, 622 aa, Artificial Sequence
  Description of Artificial Sequence Synthetic polypeptide
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASRASVGVGTGELFFGEGSRLTVLEDLKNVFPPEVAVFEPSKAEIAHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRGRAKRSGSGAPVKQTLNFDLLKLAGDVESNPGPMTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFTYGGSQGNLIFGKGTKLSVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 210 - protein, 621 aa, Artificial Sequence
  Description of Artificial Sequence Synthetic polypeptide
MGTSLLCWVVLGFLGTDHTGAGVSQSPRYKVTKRGQDVALRCDPISGHVSLYWYRQALGQGPEFLTYFNYEAQQDKSGLPNDRFSAERPEGSISTLTIQRTEQRDSAMYRCASSPELAGPQETQYFGPGTRLLVLEDLNKVFPPEVAVFEPSKAEIAHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFRAKRSGSGAPVKQTLNFDLLKLAGDVESNPGPMLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEGGGSQGNLIFGKGTKLSVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 211 - protein, 618 aa, Artificial Sequence
  Description of Artificial Sequence Synthetic polypeptide
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSGPGQGHNQPQHFGDGTRLSILEDLNKVFPPEVAVFEPSKAEIAHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFRAKRSGSGAPVKQTLNFDLLKLAGDVESNPGPMLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALGSSGTYKYIFGTGTRLKVLANIQNPDPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 212 - protein, 611 aa, Artificial Sequence
  Description of Artificial Sequence Synthetic polypeptide
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPQGRINSPLHFGNGTRLTVTEDLNKVFPPEVAVFEPSKAEIAHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFRAKRSGSGAPVKQTLNFDLLKLAGDVESNPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVRDTGYGQNFVFGPGTRLSVLPYIQNPDPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 213 - protein, 618 aa, Artificial Sequence
  Description of Artificial Sequence Synthetic polypeptide
MLLLLLLLGPGISLLLPGSLAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSAREGYRSYFGPGTRLTVLEDLKNVFPPEVAVFEPSKAEIAHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRGRAKRSGSGAPVKQTLNFDLLKLAGDVESNPGPMRLVARVTVFLTFGTIIDAKTTQPPSMDCAEGRAANLPCNHSTISGNEYVYWYRQIHSQGPQYIIHGLKNNETNEMASLIITEDRKSSTLILPHATLRDTAVYYCIVRDRSYGQNFVFGPGTRLSVLPYIQNPDPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 214 - protein, 630 aa, Artificial Sequence
  Description of Artificial Sequence Synthetic polypeptide
MLLLLLLLGPGISLLLPGSLAGSGLGAVVSQHPSWVICKSGTSVKIECRSLDFQATTMFWYRQFPKQSLMLMATSNEGCKATYEQGVEKDKFLINHASLTLSTLTVTSAHPEDSSFYICSTAGETDTQYFGPGTRLTVLEDLKNVFPPEVAVFEPSKAEIAHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRGRAKRSGSGAPVKQTLNFDLLKLAGDVESNPGPMAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAETLGLDQGGKLIFGQGTELSVKPNIQNPDSVKPNPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 215 - protein, 614 aa, Artificial Sequence
  Description of Artificial Sequence Synthetic polypeptide
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQGTGRGNTEAFFGQGTRLTVVEDLNKVFPPEVAVFEPSKAEIAHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFRAKRSGSGAPVKQTLNFDLLKLAGDVESNPGPMRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGPYFGGGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 216 - protein, 629 aa, Artificial Sequence
  Description of Artificial Sequence Synthetic polypeptide
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLGQGAVGTDTQYFGPGTRLTVLEDRKTLKNVFPPEVAVFEPSKAEIAHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRGRAKRSGSGAPVKQTLNFDLLKLAGDVESNPGPMTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFMKPDGSGNTGKLIFGQGTTLQVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 217 - protein, 19 aa, Human papillomavirus 16
TPTLHEYMLDLQPETTDLY

SEQ ID NO: 218 - protein, 9 aa, Human papillomavirus 16
SAFRCFIVY

SEQ ID NO: 219 - protein, 16 aa, Artificial Sequence
  Description of Artificial Sequence Synthetic peptide
CLVGPYFGGGSYQLTF

SEQ ID NO: 220 - protein, 16 aa, Artificial Sequence
  Description of Artificial Sequence Synthetic peptide
CASSQGTGRGNTEAFF

SEQ ID NO: 221 - protein, 14 aa, Artificial Sequence
  Description of Artificial Sequence Synthetic peptide
CAVREKGTGNQFYF

SEQ ID NO: 222 - protein, 15 aa, Artificial Sequence
  Description of Artificial Sequence Synthetic peptide
CASSLPQGTPETQYF

SEQ ID NO: 223 - protein, 14 aa, Artificial Sequence
  Description of Artificial Sequence Synthetic peptide
CGLSWGDKTDKLIF

SEQ ID NO: 224 - protein, 17 aa, Artificial Sequence
  Description of Artificial Sequence Synthetic peptide
CASSPSLAGVVPGELFF

SEQ ID NO: 225 - protein, 17 aa, Artificial Sequence
  Description of Artificial Sequence Synthetic peptide
CAFMKPDGSGNTGKLIF

SEQ ID NO: 226 - protein, 17 aa, Artificial Sequence
  Description of Artificial Sequence Synthetic peptide
CASSLGQGAVGTDTQYF

SEQ ID NO: 227 - DNA, 9374 bp, Artificial Sequence
  Description of Artificial Sequence Synthetic polynucleotide
aagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatgcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgatatcctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctctagataatacgactcactatagggcccgggttggatccgccgccaccatgggctgcagactgctgtgctgtgtggtgttctgcctgctgcaagccggacctctggatacagccgtgtctcagacccctaagtacctggtcacccagatgggcaacgacaagagcatcaagtgcgagcagaacctgggccacgacaccatgtactggtacaagcaggacagcaagaaattcctgaagatcatgttcagctacaacaacaaagagctgatcatcaacgagacagtgcccaaccggttcagccctaagagccctgataaggcccacctgaacctgcacatcaacagcctggaactgggcgacagcgccgtgtacttttgtgccagctctcaaggcaccggcagaggcaataccgaggccttttttggccaaggcacccggctgaccgtggtggaagatctgaacaaggtgttccctccagaggtggccgtgttcgagccttctaaggccgagattgcccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggaattaccagcgccagctaccatcagggcgtgctgtctgccacaatcctgtacgagatcctgctgggcaaagccactctgtacgccgtgctggtgtctgccctggtgctgatggccatggtcaagcggaaggacttcagagccaagagatccggctccggcgcaccggtgaaacagactttgaattttgaccttctcaagttggcaggagacgttgagtccaaccctgggcccatgagacaggtggccagagtgatcgtgttcctgacactgagcaccctgagcctggccaagaccacacagcccatcagcatggacagctacgagggccaagaagtgaacatcacctgtagccacaacaatatcgccaccaacgactacatcacgtggtatcagcagttccccagtcaaggccccagattcatcatccaaggctacaagaccaaagtgaccaacgaggtggcctctctgttcatccccgccgacagaaagagcagcaccctgtctctgcctagagtgtccctgagcgataccgccgtgtactactgtctcgtgggcccttattttggcggcggaagctaccagctgaccttcggcaagggcacaaagctgagcgtgatccccaacattcagaaccccgatcctgccgtgtaccagctgcgggatagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttccccagctccgatgtgccctgcgacgtgaagctggtggaaaagagcttcgagacagacaccaacctgaacttccagaacctgtccgtgatcggcttccgcatcctgctgctgaaagtggccggcttcaacctgctgatgaccctgagactgtggtccagctaatgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgc

SEQ ID NO: 228 - DNA, 9419 bp, Artificial Sequence
  Description of Artificial Sequence Synthetic polynucleotide
aagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatgcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgatatcctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctctagataatacgactcactatagggcccgggttggatccgccgccaccatgggcaccagactgctgttctgggtcgccttttgtctgctgggcgccgatcatacaggtgccggtgtttctcagagccccagcaacaaagtgaccgagaagggcaaagacgtggaactgagatgcgaccccatcagcggacacacagccctgtactggtacagacagtctctcggccagggcctcgagttcctgatctacttccaaggcaacagcgcccctgacaagagcggcctgcctagcgatagattcagcgccgaaagaacaggcggcagcgtgtccacactgaccatccagagaacccagcaagaggacagcgccgtgtacctgtgtgccagctctcttggacaaggcgccgtgggcacagacacccagtattttggccctggcaccaggctgaccgtgctggaagatagaaagaccctgaagaacgtgttcccacctgaggtggccgtgttcgagccttctaaggccgagattgcccacacacagaaagccacactcgtgtgtctggccaccggcttctatcccgaccatgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggaattaccagcgccagctaccatcagggcgtgctgtctgccacaatcctgtacgagatcctgctgggcaaagccactctgtacgccgtgctggtgtctgccctggtgctgatggccatggtcaagcggaaggatagcagaggcagagccaagagatccggctccggcgcaccggtgaaacagactttgaattttgaccttctcaagttggcaggagacgttgagtccaaccctgggcccatgaccagagtgtctctgctgtgggccgtcgtggtgtccacatgtctggaatctggcatggcccagaccgtgacacagagccagcctgagatgtctgtgcaagaggccgagacagtgaccctgagctgcacctacgataccagcgagaacaactactacctgttctggtacaagcagcctcctagccggcagatgatcctggtcatcagacaagaggcctataagcagcagaacgccaccgagaacagattcagcgtgaacttccagaaggccgccaagagcttcagcctgaagatcagcgatagccagctgggcgacaccgccatgtacttttgcgcctttatgaagcccgacggcagcggcaataccggcaagctgatttttggccagggcaccacactgcaagtgaagcccaacattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttccccagctccgatgtgccctgcgacgtgaagctggtggaaaagagcttcgagacagacaccaacctgaatttccagaacctgagcgtgatcggcttccgcatcctgctgctgaaagtggccggcttcaacctgctgatgaccctgcgactttggagcagctaatgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgc

SEQ ID NO: 229 - protein, 292 aa, Artificial Sequence
  Description of Artificial Sequence Synthetic polypeptide
DTAVSQTPKYLVTQMGNDKSIKCEQNLGHDTMYWYKQDSKKFLKIMFSYNNKELIINETVPNRFSPKSPDKAHLNLHINSLELGDSAVYFCASSQGTGRGNTEAFFGQGTRLTVVEDLNKVFPPEVAVFEPSKAEIAHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 230 - protein, 254 aa, Artificial Sequence
  Description of Artificial Sequence Synthetic polypeptide
LAKTTQPISMDSYEGQEVNITCSHNNIATNDYITWYQQFPSQGPRFIIQGYKTKVTNEVASLFIPADRKSSTLSLPRVSLSDTAVYYCLVGPYFGGGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 231 - protein, 299 aa, Artificial Sequence
  Description of Artificial Sequence Synthetic polypeptide
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLGQGAVGTDTQYFGPGTRLTVLEDRKTLKNVFPPEVAVFEPSKAEIAHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 232 - protein, 259 aa, Artificial Sequence
  Description of Artificial Sequence Synthetic polypeptide
AQTVTQSQPEMSVQEAETVTLSCTYDTSENNYYLFWYKQPPSRQMILVIRQEAYKQQNATENRFSVNFQKAAKSFSLKISDSQLGDTAMYFCAFMKPDGSGNTGKLIFGQGTTLQVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 233 - protein, 4 aa, Artificial Sequence
  Description of Artificial Sequence Synthetic peptide
SGSG

(19) United States
(12) Patent Application Publication
(10) Pub. No.: US 2023/0348558 A1
(43) Pub. Date: Nov. 2, 2023

(54) COMPOSITIONS AND METHODS FOR TARGETING HPV-INFECTED CELLS

(71) Applicant: THE COUNCIL OF THE QUEENSLAND INSTITUTE OF MEDICAL RESEARCH, Herston Qld (AU)

(72) Inventors: Jacqueline BURROWS, Albany Creek Qld (AU); Kunal H. BHATT, Windsor Qld (AU); Rajiv KHANNA, Herston Qld (AU)

(73) Assignee: THE COUNCIL OF THE QUEENSLAND INSTITUTE OF MEDICAL RESEARCH, Herston Qld (AU)

(21) Appl. No.: 17/913,937

(22) PCT Filed: Mar. 22, 2021
(86) PCT No.: PCT/IB2021/000158

§ 371 Date: Sep. 23, 2022

Related U.S. Application Data
(60) Provisional application No. 62/993,442, filed on Mar. 23, 2020.

(51) Int. Cl. C07K 14/725; C12N 15/86; C12N 5/0783

(52) U.S. Cl. CPC C07K 14/7051; C12N 15/86; C12N 5/0638; C07K 2317/565; C12N 2740/15043

(57) ABSTRACT

Disclosed are compositions and methods for targeted treatment of cancer, such as HPV-associated cancer. In particular, modified T cell receptor (TCR) T cells are disclosed that can be used with adoptive cell transfer to target and kill cancer cells with reduced antigen escape. Therefore, also disclosed are methods of providing an anti-tumor immunity in a subject with HPV-associated cancer that involves adoptive transfer of the disclosed TCR-T cells.

### RELATED APPLICATIONS

This application claims the benefit of priority to U.S. Provisional Patent Application Ser. No. 62/993,442, filed Mar. 23, 2020, which is incorporated by reference in its entirety.

### BACKGROUND

Adoptive cell transfer (ACT) involves implanting or infusing particular cells, typically immune cells and/or cells derived from the immune system (e.g., sensitized, modified, and/or engineered lymphocytes), into a patient with the aim of recognizing, targeting, and/or destroying disease-associated cells. Adoptive immunotherapies (e.g., T cell therapies) have become a promising approach for the treatment of many diseases and disorders, including post-transplant lymphoproliferative disorders, infectious diseases (e.g., viral infections), and autoimmune diseases. Adoptive T cell therapy is also a promising cancer treatment modality, showing encouraging results in clinical trials. Infusion of tumor-infiltrating lymphocytes (TIL), isolated from metastatic tumors of the patient and expanded ex vivo have been associated with complete regression of metastatic melanoma and cervical cancer in some patients. However, manufacturing of such autologous adoptive T cell therapies is often time-consuming and of limited use to terminally ill patients. Thus, there exists an urgent and unmet need to develop an “off-the-shelf” strategy for rapid delivery of adoptive T cell therapy. This can potentially be achieved by genetically engineering lymphocytes (e.g., T cells such as cytotoxic T cells) to express a tumor or disease-targeting moiety, such as a chimeric antigen receptor (CAR) or an engineered T cell receptor (TCR). This strategy has shown encouraging early results in clinical trials for B-cell malignancies, melanoma, and synovial cell sarcoma. However, efforts to extend this approach to epithelial cancers have generally relied on targets (e.g., antigens) that are shared by both tumors and healthy tissues, and are thus limited by T cell-mediated on-target, off-tumor toxicity. Such off-tumor toxicity might be avoided by targeting a tumor antigen that is not expressed by healthy tissues, but few antigens are both exclusive to malignant cells and expressed commonly by a particular family of epithelial cancers.

Selective infection of skin or mucous membranes is a classic feature of HPVs, and their replication is closely linked to the maturation of the cells in these membranes. Globally, HPV infection accounts for an estimated 530,000 cervical cancer cases (˜270,000 deaths) annually, with the majority (86% of cases, 88% of deaths) occurring in developing countries. In total, HPV accounts for 5.2% of the worldwide cancer burden. Each year in the United States, an estimated 26,000 new cancers are attributable to HPV, about 17,000 in women and 9,000 in men. The most common HPV types are the low-risk HPV-6 and HPV-11, which are responsible for 90% of genital warts and a disease known as recurrent respiratory papillomatosis, in which tumors grow in the airway. HPV also plays a role in the development of non-melanoma skin cancer (NMSC), including cutaneous squamous cell carcinoma (SCC), among chronic lymphocytic leukemia (CLL) and blood and marrow transplant (BMT) patients. HPV-16 and HPV-18, in particular, account for the majority of head and neck cancers (HNSCCs) and cancers of the cervix, anus, vagina, vulva, penis, tongue base, larynx, and tonsil. Current standard therapeutic options for HNSCCs include and incorporate surgery and radiotherapy with concurrent chemotherapy (e.g., cisplatin and/or cetuximab). Unfortunately, the post-treatment burden on the patient following such modalities can be significant and permanent and may include dysphagia, dysphonia, xerostomia, scarring and disfigurement, and trismus. However, HPV-associated precancerous lesions, such as those of the vulva, vagina, anus, penis, as well as genital warts, are typically treated using physical elimination by cryotherapy (i.e., using extreme cold to destroy tissue), chemical cauterization (i.e., using a chemical to destroy tissue), and laser or surgical removal. Notably, in such pre-cancerous lesions physical elimination alone is not very effective, since 20-30% or more cases recur, with lesions both at previously treated sites due to failure of the procedure to eliminate the HPV, and at new sites due to new infections. When this occurs, radiotherapy and chemotherapy are then used with relative success; however, about 50% of the HPV-associated cancer patients still die of the disease. Clearly, new treatment strategies are urgently needed to control the burden of HPV-related cancer.

### SUMMARY

The present invention is based, at least in part, on the discovery that HPV antigens, particularly those apart from E6 and E7, can be used for the targeted treatment of HPV-associated diseases (e.g., HPV-associated cancers such as head and neck, gastrointestinal, genitourinary, and gynecologic cancers). In some aspects, provided herein are immune cells that express an engineered TCR (e.g., TCR-T cells, TCR-Ts) that target HPV antigens.

Aspects of the invention disclosed herein include a T cell receptor (TCR) polypeptide having antigenic specificity for human papillomavirus (HPV) 16. In some embodiments, said TCR comprises at least one complementary determining region 3α (CDR3α) and at least one CDR3β amino acid sequence selected from the amino acid sequences set forth in Tables 1 and/or 13. In some embodiments of the invention, TCR polypeptides disclosed herein are specific for antigens comprising at least one epitope having an amino acid sequence selected from the amino acid sequences set forth in Tables 1, 11 and/or 13, e.g., SEQ ID NOs: 1-12, 217, or 218.

In certain aspects of the invention, disclosed herein are methods of treating a cancer or precancerous lesions in a subject, the method comprising administering an effective amount of an adoptive immunotherapy composition comprising the cells expressing the TCR polypeptides contemplated herein. In preferred embodiments, the cancer or lesion is an HPV-associated cancer or lesion. In some aspects of the invention, disclosed herein are cell banks comprising cells for adoptive immunotherapy. In some embodiments, the cells of such banks express the TCRs contemplated herein. In certain preferred embodiments, the HLA restriction of said TCR-expressing cells is known. In certain embodiments, treating an HPV-associated cancer or precancerous lesion, as disclosed herein, comprises administering an effective amount of an adoptive immunotherapy composition comprising TCR-expressing cells selected from the cell banks contemplated herein.

### BRIEF DESCRIPTION OF THE DRAWINGS

FIG. 1 shows the sorting path of HPV16-E5-NLD epitope specific CD8⁺ T cells detected by a dual cytokine capture assay, and sorted by double positive (IFNγ and TNFα) single cells into 96 well PCR plates.

FIG. 2 shows an agarose gel electrophoresis image of TCR segments containing CDR3α and CDR3β. Briefly, RT-PCR is performed on individual cells and the resultant cDNA is subjected to two rounds of nested PCR. In the first round, TCRα and TCRβ transcript amplification is achieved with a multiplexed, comprehensive panel of external, sense Vα and Vβ segment-specific primers and antisense Cα and Cβ segment-specific primers. The first-round PCR products are subjected to two separate second-round PCRs, incorporating, respectively, (1) a multiplexed panel of external sense Vα and antisense Cα segment-specific primers or (2) external sense Vβ and antisense Cβ segment-specific primers. Paired TCRα and TCRβ products from the same cell were loaded in adjacent lanes and shown in paired, labeled columns. Negative control (H1-H12) PCR reactions are shown in the bottom row (right lanes). In the ladder lane, a 300 bp label is shown.

FIG. 3 shows the E2-TLQ-TCR amino acid sequence (SEQ ID NO. 209), indicating relevant features.

FIG. 4 shows the E5-NLD-TCR amino acid (SEQ ID NO. 210), indicating relevant features.

FIG. 5 shows an exemplary lentiviral construct (E5-NLD-TCR), indicating relevant features.

FIG. 5 discloses “SGSG linker” as SEQ ID NO: 233.

FIG. 6 shows the E6-AFR-TCR amino acid sequence (SEQ ID NO. 211), indicating relevant features.

FIG. 7 shows the E6-TIH-TCR amino acid sequence (SEQ ID NO. 212), indicating relevant features.

FIG. 8 shows the E6-HDI-TCR amino acid sequence (SEQ ID NO. 213), indicating relevant features.

FIG. 9 shows the E6-KQR-TCR amino acid sequence (SEQ ID NO. 214), indicating relevant features.

FIG. 10 shows the E7-TPT-TCR amino acid sequence (SEQ ID NO. 215), indicating relevant features.

FIG. 11 shows the E5-SAF-TCR amino acid sequence (SEQ ID NO. 216), indicating relevant features.

FIG. 12, A-C, shows lentiviral transfer of HPV-specific TCR into Jurkat cells, TCR expression confirmed by flow cytometry. Dot plots show the TCR expression in nontransduced controls and Jurkat cells transduced with (A) E2-TLQ-TCR, (B) E5-NLD-TCR, and (C) E6-TIH-TCR

FIGS. 13, A and B, shows lentiviral transfer of HPV-specific TCR into Jurkat cell confers antigen specificity. Lentiviral TCR-transduced Jurkat cells were co-incubated with peptide-pulsed, HLA-matched or mismatched LCLs. After 24 hours, CD69 expression was checked by flow cytometry. Cytometric analysis shows (A) E5-NLD-lentiTCR (restricted to HLA-C*05:01 & C*08:02) and (B) E2-TLQ-lentiTCR (restricted to HLA A*02:01) transduced Jurkat cell express CD69.

FIG. 14 shows lentiviral transfer of HPV-specific TCR into PBMCs confers TCR expression. Briefly, lentiviral transduction of PBMC was performed 48 h post stimulation. At day 8 and day 15 (day 7 after re-stimulation) TCR expression was assessed by flow cytometry. Representative data is shown for E5-NLD-TCR-transduced PBMC TCR expression, i.e., anti-TCRVα12.1-positive, CD8⁺ cells.

FIG. 15 shows antigenic specificity of transgenic TCR assessed by multiparametric intracellular cytokine staining (ICS) assay. Briefly, nontransduced and E5-NLD-lentiviral-transduced PBMCs were stimulated with the E5 antigen peptide (NLD peptide; SEQ ID NO. 1) and incubated for 5 hours. Dot plots show CD107, IFNγ, TNFα and IL-2 expression in CD8⁺ cells and CD4⁺ cells (last row).

FIG. 16 shows avidity of TCR-T cells for cognate antigen measured by recall ICS assay. Transduced and nontransduced PBMC were stimulated with HLA-matched LCL pulsed with different concentration of peptide (10⁻⁶, 10⁻⁷, 10⁻⁸, 10⁻⁹, 10⁻¹⁰, 10⁻¹¹, 10⁻¹² and 10⁻¹³ mole/L) for 4 hours and IFNγ expression was measured by ICS assay. The line graph is showing E5-NLD lentivirus transduced PBMC IFNγ expression after stimulating with HLA matched (C*05:01 and C*08:02) LCL.

FIG. 17, A-C, shows cytolysis for CaSki cell line (HPV16⁺ and HLA-A*02:01⁺) with E2-TLQ-T (A), cytolysis for CaSki cell line (HPV16⁺ and HLA-A*02:01 +ve) with untransduced T cells (B), and cytolysis for SCC70 cell line (HPV16-ve and HLA-A*02:01⁺) with E2-TLQ-T and untransduced T cells (C). UT=untransduced T cells; E2 TCR=E2-TLQ-TCR-transduced T cells.

### DETAILED DESCRIPTION

General

The use of engineered TCR therapy provides several advantages. The patients' own T cells may be equipped with desired specificities and allow generation of sufficient numbers of T cells in a short period of time while avoiding T cell exhaustion. In some embodiments of the invention, as disclosed herein, TCRs may be transduced into immune effector cells such as central memory T cells or T cells with stem cell characteristics, which may ensure better persistence and function upon transfer. Preferably, TCRs are transduced into cytotoxic T cells (CD8⁺ T cells; CTLs). Such TCR-engineered T cells (TCR-T cells) can be infused into cancer patients, such as cancer patients rendered lymphopenic by chemotherapy or irradiation, allowing for efficient engraftment but inhibiting immune suppression. As disclosed herein, the present invention relates, at least in part, to immune cells which recombinantly express an artificial T cell receptor (TCR) that targets HPV antigens.

Notably, HPV oncoproteins E6 and E7 are constitutively expressed and important for the survival of HPV-associated cancers but are absent from healthy tissues. In at least HNSCCs, HPV16 has been reported to integrate into the host genome leading to disruption of E2 early genes, which is a negative regulator of E6 and E7 oncogene expression, thus making E6 and E7 antigens the primary focus of research. However, recent cancer genome sequencing in HNSCCs suggests that the majority of such cancers (e.g., tumors) that contain hybrid episomal forms of HPV, comprise other HPV antigens, apart from E6 and E7. In addition, higher E2 expression represses E6 and E7 expression, further changing the expression profile of HPV antigens and subsequent immune T cell infiltration within tumors. Moreover, adoptive T cell therapies directed against HPV oncoproteins have typically been restricted to HLA-A*02:01 which is the most common class I allele in the United States, expressed in approximately 40-50 percent of people of European descent. Unfortunately, HPV infection can selectively downregulate HLA-A and HLA-B from the surface of infected cells without affecting HLA-C expression. Thus, other early proteins might be targeted instead of, or in combination with, E6 and E7 antigens through adoptive T cell therapy (e.g., TCR-T therapy), offering better efficacy than targeting E6 and E7 alone. Likewise, TCR sequences specific for different early antigens (e.g., other than, or in addition to, E6 and E7 antigens) and restricted to HLA B and HLA-C alleles may provide better efficacy and prognosis of HPV-associated disease, including various cancers. Accordingly, aspects of the invention disclosed herein include TCRs that specifically target HPV16 antigens, preferably early HPV16 antigens E1, E2, E4, and E5, with restriction to HLA-A, HLA-B and HLA-C alleles. Also provided herein are immune effector cells (e.g., T cells) genetically engineered to express said antigen-specific TCRs (e.g., TCR-Ts).

In some embodiments, TCR-T cells, as described herein, are engineered to counteract any tolerogenic effects of the malignant cellular microenvironment (e.g., a tumor microenvironment) by, for example and without limitation, suppressing or inhibiting PD-1 signaling. In certain embodiments, the TCRs described herein may be sensitized to or selectively target a viral or non-viral antigen. An ideal target should not be expressed on any normal tissue/organ, or at least not in vital normal tissues (heart, liver, CNS, lung, and other tissues that may be particularly sensitive to transient damage) nor in closely related normal cellular counterparts (e.g., stem and/or progenitor cells), in order to minimize side effects (e.g., on target/off tumor or bystander effects). Also disclosed herein are immune effector cells, such as T cells or Natural Killer (NK) cells, that are engineered to express recombinant TCR polypeptides that selectively bind HPV antigens (e.g., wildtype and/or mutant HPV16). Therefore, also disclosed are methods for providing targeted immunity (e.g., anti-tumor immunity) in a subject with an HPV-associated disease or malignancy that involves adoptive transfer of the disclosed immune effector cells engineered to express the disclosed TCR polypeptides.

In the tumor microenvironment cancer cells and host immune cells interact, potentially leading to promotion or inhibition of cancer progression. Ideally, the immune system would identify cancer cells and mobilize an immune response to eliminate the cancer. Unfortunately, at the T cell level, upregulation of inhibitory receptors, such as PD-1 and Tim-3, correlates with T cell dysfunction. This has been observed on both hepatitis C virus (HCV)-specific and HCV-nonspecific CD8⁺ T cells in the circulation and livers of patients with chronic HCV infection. Partial restoration of T cell proliferation and IFN-7 secretion can be achieved ex vivo by inhibiting the binding of PD-1 and Tim-3 to their respective ligands (i.e., B7-H1, also known as PD-L1, and Galectin-9). What is more, recent reports have demonstrated that prolonged administration of IFN-α, a standard therapy for persistent HCV infection, promoted telomere loss in naïve T cells. Given the correlation between shortened T cell telomeres and terminal differentiation (characterized by diminished proliferative potential), IFN-α-induced T cell “exhaustion” likely represents a significant barrier for immunotherapy in HCV-infected patients. In certain aspects disclosed herein, the invention employs checkpoint inhibition strategies. Checkpoint inhibitor therapies target key regulators of the immune system that either stimulate or inhibit the immune response. Such immune checkpoints can be exploited in the cancer disease state (e.g., by tumors) to evade attacks by the immune system. Checkpoint inhibitor studies have noted the activity of PD-1 inhibitor therapy (El-Khoueiry et al., (2017). “Nivolumab in patients with advanced hepatocellular carcinoma (CheckMate 040): an open-label, non-comparative, phase 1/2 dose escalation and expansion trial.” Lancet 389 (10088): 2492-2502) and the FDA has approved Nivolumab for second line treatment of HCC with an objective response rate of 20%.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering. Such an agent can contain, for example, peptide described herein, an antigen presenting cell provided herein and/or a CTL provided herein.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of the foregoing.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as VH) and a heavy chain constant region. Each light chain includes a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments.

The terms “antigen-binding fragment” and “antigen-binding portion” of an antibody, as used herein, refers to one or more fragments of an antibody that retain the ability to bind to an antigen. Examples of binding fragments encompassed within the term “antigen-binding fragment” of an antibody include Fab, Fab′, F(ab′)2, Fv, scFv, disulfide linked Fv, Fd, diabodies, single-chain antibodies, camelid antibodies, isolated CDRH3, a Designed Ankyrin Repeat Protein (DARPin) and other antibody fragments that retain at least a portion of the variable region of an intact antibody. These antibody fragments can be obtained using conventional recombinant and/or enzymatic techniques and can be screened for antigen binding in the same manner as intact antibodies.

The term “antigen binding site” refers to a region of an antibody or T cell that specifically binds the epitope(s) of an antigen.

The term “binding” or “interacting” refers to an association, which may be a stable association, between two molecules, e.g., between a peptide and a binding partner or agent, e.g., small molecule, due to, for example, electrostatic, hydrophobic, ionic and/or hydrogen-bond interactions under physiological conditions.

The term “biological sample,” “tissue sample,” or simply “sample” each refers to a collection of cells obtained from a tissue of a subject. The source of the tissue sample may be solid tissue, as from a fresh, frozen and/or preserved organ, tissue sample, biopsy, or aspirate; blood or any blood constituents, serum, blood; bodily fluids such as cerebral spinal fluid, amniotic fluid, peritoneal fluid or interstitial fluid, urine, saliva, stool, tears; or cells from any time in gestation or development of the subject.

As used herein, the term “cancer” includes, but is not limited to, solid tumors and blood borne tumors. The term cancer includes diseases of the skin, tissues, organs, bone, cartilage, blood, and vessels, including the cervix, anus, vagina, vulva, penis, tongue base, larynx, and tonsil. The term “cancer” further encompasses primary and metastatic cancers.

The term “chimeric molecule” refers to a single molecule created by joining two or more molecules that exist separately in their native state. The single, chimeric molecule has the desired functionality of all of its constituent molecules. One type of chimeric molecules is a fusion protein.

The term “epitope” means a protein determinant capable of specific binding to an antibody or immune cell (e.g., T cell). Epitopes usually consist of chemically active surface groupings of molecules such as amino acids or sugar side chains. Certain epitopes can be defined by a particular sequence of amino acids to which a T cell receptor (TCR) or antibody is capable of binding.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides, or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc.), may be transfected into cells, e.g., mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically-acceptable carrier” means a pharmaceutically-acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The terms “polynucleotide”, and “nucleic acid” are used interchangeably. They refer to a natural or synthetic molecule, or some combination thereof, comprising a single nucleotide or two or more nucleotides linked by a phosphate group at the 3′ position of one nucleotide to the 5′ end of another nucleotide. The polymeric form of nucleotides is not limited by length and can comprise either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides. The polynucleotide is not necessarily associated with the cell in which the nucleic acid is found in nature, and/or operably linked to a polynucleotide to which it is linked in nature.

The term “polypeptide” or “isolated polypeptide” refers to a polypeptide, in certain embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In other embodiments, a fragment may have immunogenic properties.

The term “precancerous lesions” or “precancerous condition” refers to atypical cells and/or tissues that are associated with an increased risk of cancer. The term “precancerous lesions” may refer, for example, to dysplasia, benign neoplasia, or carcinoma in situ.

As used herein, a therapeutic that “prevents” a condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

As used herein, “specific binding” refers to the ability of an antibody or TCR to bind to a predetermined antigen or the ability of a peptide to bind to its predetermined binding partner. Typically, an antibody or peptide specifically binds to its predetermined antigen or binding partner with an affinity corresponding to a KD of about 10-7 M or less, and binds to the predetermined antigen/binding partner with an affinity (as expressed by KD) that is at least 10 fold less, at least 100 fold less or at least 1000 fold less than its affinity for binding to a non-specific and unrelated antigen/binding partner (e.g., BSA, casein).

A “spacer” as used herein refers to a peptide that joins the proteins comprising a fusion protein. Generally, a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically binds” or “specific binding”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g., immunoassay conditions in the case of an antibody), a specified ligand or antibody “specifically binds” to its particular “target” (e.g., an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule. For example, in the case of the ability of a TCR to bind to a peptide presented on an MHC (e.g., class I MHC or class II MHC); typically, a TCR specifically binds to its peptide/MHC with an affinity of at least a KD of about 10-4 M or less, and binds to the predetermined antigen/binding partner with an affinity (as expressed by KD) that is at least 10 fold less, at least 100 fold less or at least 1000 fold less than its affinity for binding to a non-specific and unrelated peptide/MHC complex (e.g., one comprising a BSA peptide or a casein peptide).

As used herein, the term “subject” means a human or non-human animal selected for treatment or therapy.

The terms “transformation”, “transfection”, or “transduction” mean the introduction of a nucleic acid, e.g., an expression vector, into a recipient cell (e.g., a mammalian cell) including introduction of a nucleic acid to the chromosomal DNA of said cell.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated,” for example, if one or more symptoms associated with a particular disease, disorder, or condition are mitigated or eliminated.

The term “variant” refers to an amino acid or peptide sequence having conservative amino acid substitutions, non-conservative amino acid substitutions (e.g., a degenerate variant), substitutions within the wobble position of each codon (e.g., DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

The term “vector” refers to the means by which a nucleic acid can be propagated and/or transferred between organisms, cells, or cellular components. Vectors include plasmids, viruses, bacteriophage, pro-viruses, phagemids, transposons, and artificial chromosomes, and the like, to which the nucleic acid has been linked, and may or may not be able to replicate autonomously or integrate into a chromosome of a host cell. Such vectors may include any vector, (e.g., a plasmid, cosmid or phage chromosome) containing a gene construct in a form suitable for expression by a cell (e.g., linked to a transcriptional control element).

In certain embodiments, agents of the invention may be used alone or conjointly administered with another type of therapeutic agent. As used herein, the phrase “conjoint administration” or “administered conjointly” refers to any form of administration of two or more different therapeutic agents (e.g., a composition comprising a TCR-T cell disclosed herein and an inhibitor of an immune checkpoint) such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In some preferred embodiments, the TCR-T cells express (e.g., present on the cell surface or secrete) further therapeutic agents. In certain embodiments, the different therapeutic agents (e.g., TCR-Ts and immune checkpoint-blocking molecules) can be administered within about one hour, about 12 hours, about 24 hours, about 36 hours, about 48 hours, about 72 hours, or about a week of one another. Similarly, in some embodiments, such compositions as are described herein may be used conjointly in a therapeutic regimen combined with other treatments, therapies, or interventions appropriate for the particular disease, condition, injury or disorder being treated. Thus, the therapeutic agents and compositions of the invention can be administered either concomitantly or sequentially in combination with one or more treatment modalities, e.g., chemotherapy, radiotherapy, surgery, or any combination thereof. For example and without limitation, the different therapeutic agents and compositions of the invention (e.g., TCR-Ts alone or in combinations with immune checkpoint-blocking molecules) can be administered during, within about one hour, within about 12 hours, within about 24 hours, within about 36 hours, within about 48 hours, within about 72 hours, or within about a week of chemotherapy, radiotherapy, or surgery. Thus, a subject who receives such treatment can benefit from a combined effect of different therapeutic agents and modalities.

T Cell Receptors (TCRs)

A TCR is a heterodimeric cell-surface protein of the immunoglobulin super-family which is associated with invariant proteins of the CD3 complex involved in mediating signal transduction. TCRs exist in αβ and γδ forms, which are structurally similar but have distinct anatomical locations and functions. The extracellular domains of native αβTCRs consist of two polypeptides (an α-chain and a β-chain), each of which comprise a membrane-proximal constant domain, and a membrane-distal variable domain, each of which include an intra-chain disulfide bond. A short segment, analogous to an immunoglobulin hinge region, connects the immunoglobulin-like domains to the membrane (via the transmembrane region) and contains the cysteine residue that forms an interchain disulfide bond.

The variable domain contains the highly polymorphic loops referred to as complementarity determining regions (CDRs) which are responsible for binding to the peptide-presenting major histocompatibility complex (MHC). Within the variable domain, each α-chain and β-chain of a native heterodimeric αβTCR comprises variable, joining, and constant regions; the β-chain also usually contains a short diversity region between the variable and joining regions, but this diversity region is often considered as part of the joining region. Each variable region comprises three CDRs (Complementarity Determining Regions) embedded in a framework sequence, one being the hyper-variable region named CDR3, the main CDR responsible for recognizing the antigen presented on the MHC. There are several types of α-chain variable (Vα) regions and several types of β-chain variable (Vβ) regions distinguished by their framework, CDR1 and CDR2 sequences, and by a partly defined CDR3 sequence.

In some aspects of the invention, provided herein are engineered TCRs that can be expressed in immune effector cells to enhance activity against specific targets (e.g., antitumor activity). The TCR sequences provided herein are capable of specifically binding antigenic peptides comprising HPV epitopes (i.e., have antigenic specificity). T cells (e.g., cytotoxic T cells; CTLs) expressing such engineered TCRs are useful in the prevention and/or treatment of HPV infection, and/or cancer (e.g., a cancer expressing an HPV epitope), and/or precancerous lesions. In some embodiments, the TCR sequence (and the HPV epitope sequence to which it specifically binds) comprises a sequence listed in Table 1. Therefore, in some embodiments, the antigen-recognizing constructs of the invention (e.g., TCRs) comprise CDR1, CDR2 and CDR3 sequences in a combination which display the respective variable chain allele together with the CDR3 sequence. Preferred embodiments of the invention comprise TCR constructs that comprise at least one or more of the CDR3s set forth in Tables 1 and 13 (e.g., SEQ ID NOs. 13 to 52, or 219 to 226), or variants thereof (e.g., having conservative substitutions in the amino acid sequence, e.g., 1-5 such conservative substitutions), but more preferably all three CDR sequences CDR1, CDR2 and CDR3. In some such embodiments, said TCR comprises at least one complementary determining region 3α (CDR3α) and at least one CDR3β amino acid sequence selected from the amino acid sequences set forth in SEQ ID NOs. 13 to 52, or 219 to 226. In certain preferred embodiments, the TCR polypeptide comprises a CDR3α amino acid sequence and CDR3β amino acid set forth in SEQ ID NOs. 13 and 14, SEQ ID NOs. 15 and 16, SEQ ID NOs. 17 and 18, SEQ ID NOs. 25 and 26, SEQ ID NOs. 27 and 28, SEQ ID NOs. 29 and 30, SEQ ID NOs. 51 and 52, SEQ ID NOs. 219 and 220, or SEQ ID NOs. 225 and 226.

In some embodiments, the TCR polypeptides disclosed herein are specific for HPV antigens, preferably HPV antigens derived from HPV peptides other than E6 and E7. In particularly preferred embodiments of the invention, the TCR polypeptides disclosed herein have antigenic specificity for any one of HPV16 peptides E1, E2, E4, E5, or combinations thereof. Most preferred, the TCR polypeptides are specific for antigens comprising at least one epitope having an amino acid sequence selected from the amino acid sequences set forth in SEQ ID NOs: 1-12, 217, or 218.

TABLE 1
HPV-specific TCR sequences

| | | HLA | Population | | TCRα and |
|---|---|---|---|---|---|
| Antigen | Epitope | restriction | Coverage | CDR3 sequence | TCRβ gene |
| HPV16-E5 | NLDTASTTL | C*05:01, | 42.40% | CDR3α sequence: | TRAV19-01 |
| | (SEQ ID NO. 1) | C*08:02 & | (15.33%, | CALSEGGGSQGNLIF | TRAJ42-01 |
| | | C*04:01 | 7.88% & | (SEQ ID NO. 13) | |
| | | | 23.24%) | CDR3β sequence: | TRBV7-6*01 |
| | | | | CASSPELAGPQETQYF | TRBJ2-5*01 |
| | | | | (SEQ ID NO. 14) | TRBD2*01 |
| | | | | CDR3α sequence: | TRAV8-4*07 |
| | | | | CAVSDRHDMRF | TRAJ43*01 |
| | | | | (SEQ ID NO. 15) | |
| | | | | CDR3β sequence: | TRBV6-5*01 |
| | | | | CASSYSPERHEQFF | TRBJ2-1*01 |
| | | | | (SEQ ID NO. 16) | TRBD2*01 |
| | | | | CDR3α sequence: | TRAV27*01 |
| | | | | CASEGHDMRF | TRAJ43*01 |
| | | | | (SEQ ID NO. 17) | |
| | | | | CDR3β sequence: | TRBV6-5*01 |
| | | | | CASSTEAGGPTGELFF | TRBJ2-2*01 |
| | | | | (SEQ ID NO. 18) | TRBD2*01 |
| HPV16-E6 | HDIILECVY | B*18:01 | 8.03% | CDR3α sequence: | TRAV26-1*01 |
| | (SEQ ID NO. 2) | (B44 super | | CIVRDRSYGQNFVF | TRAJ26*01 |
| | | family ) | | (SEQ ID NO. 19) | |
| | | | | CDR3β sequence: | TRBV20-1*01 |
| | | | | CSAREGYRSYF | TRBJ2-1*01 |
| | | | | (SEQ ID NO. 20) | TRBD1*01 |
| | | | | CDR3α sequence: | TRAV17*01 |
| | | | | CARGLENAGNMLTF | TRAJ39*01 |
| | | | | (SEQ ID NO. 21) | |
| | | | | CDR3β sequence: | TRBV27*01 |
| | | | | CATSVRGTQPQHFF | TRBJ1-5*01 |
| | | | | (SEQ ID NO. 22) | TRBD1*01 |
| | | | | CDR3α sequence: | TRAV19*01 |
| | | | | CALSERGSGGFKTIF | TRAJ9*01 |
| | | | | (SEQ ID NO. 23) | |
| | | | | CDR3β sequence: | TRBV7-8*01, |
| | | | | CASSSGEKGQGAPVSSYE | TRBJ2-7*01 |
| | | | | QYFF | TRBD1*01 |
| | | | | (SEQ ID NO. 24) | |
| HPV16-E2 | TLQDVSLEVYL | A*02:01 | 47.41% | CDR3α sequence: | TRAV38-01*03 |
| | (SEQ ID NO. 3) | | | CAFTYGGSQGNLIF | TRAJ42-01 |
| | | | | (SEQ ID NO. 25) | |
| | | | | CDR3β sequence: | TRBV2*01 |
| | | | | CASRASVGVGTGELFF | TRBJ2-2*01 |
| | | | | (SEQ ID NO. 26) | TRBD1*01 |
| | | | | CDR3α sequence: | TRAV38-1*01 |
| | | | | CALSGNMLTF | TRAJ39*01 |
| | | | | (SEQ ID NO. 27) | |
| | | | | CDR3β sequence: | TRBV11-2*03 |
| | | | | CASSEGVGQRDEQFF | TRBJ2-1*01 |
| | | | | (SEQ ID NO. 28) | TRBD2*01 |
| | | | | CDR3α sequence: | TRAV27*01 |
| | | | | CAASWEGGGADGLTF | TRAJ45*01 |
| | | | | (SEQ ID NO. 29) | |
| | | | | CDR3β sequence: | TRBV27*01 |
| | | | | CASRGQGVYRSSYNEQFF | TRBJ2-1*01 |
| | | | | (SEQ ID NO. 30) | TRBD1*01 |
| HPV16-E6 | TIHDIILECV | A*02:01 | 47.41% | CDR3α sequence: | TRAV1-2*01 |
| | (SEQ ID NO. 4) | | | CAVRDTGYGQNFVF | TRAJ26*01 |
| | | | | (SEQ ID NO. 31) | |
| | | | | CDR3β sequence: | TRBV27*01 |
| | | | | CASSPQGRINSPLHF | TRBJ1-6*02 |
| | | | | (SEQ ID NO. 32) | TRBD1*01 |
| | | | | CDR3α sequence: | TRAV26-2*01 |
| | | | | CILSAHDYKLSF | TRAJ20*01 |
| | | | | (SEQ ID NO. 33) | |
| | | | | CDR3β sequence: | TRBV10-2*01 |
| | | | | CASSQGGLNSPLHF | TRBJ1-6*02 |
| | | | | (SEQ ID NO. 34) | TRBD1*01 |
| | | | | CDR3α sequence: | TRAV14/DV4*03 |
| | | | | CAMRVAEGSQGNLIF | TRAJ42*01 |
| | | | | (SEQ ID NO. 35) | |
| | | | | CDR3β sequence: | TRBV5-8*01 |
| | | | | CASSPWGRGGSPLHF | TRBJ1-6*02 |
| | | | | (SEQ ID NO. 36) | TRBD1*01 |
| | | | | CDR3α sequence: | TRAV12-2*01 |
| | | | | CAVKFNTDQGGKLIF | TRAJ23*01 |
| | | | | (SEQ ID NO. 37) | |
| | | | | CDR3β sequence: | TRBV9*01, |
| | | | | CASSPQGRINSPLHF | TRBJ2-3*01 |
| | | | | (SEQ ID NO. 38) | TRBD2*01 |
| HPV16-E6 | AFRDLCIVY | C*07:02 | 25.32% | CDR3α sequence: | TRAV19*01 |
| | (SEQ ID NO. 5) | | | CALGSSGTYKYI | TRAJ40*01 |
| | | | | (SEQ ID NO. 39) | |
| | | | | CDR3β sequence: | TRBV25-1*01 |
| | | | | CASSGPGQGHNQPQHF | TRBJ1-5*01 |
| | | | | (SEQ ID NO. 40) | TRBD1*01 |
| | | | | CDR3α sequence: | TRAV14/DV4*03 |
| | | | | CAMREANDMRF | TRAJ43*01 |
| | | | | (SEQ ID NO. 41) | |
| | | | | CDR3β sequence: | TRBV11-2*01 |
| | | | | CASSFLVLAVSYNEQFF | TRBJ2-1*01 |
| | | | | (SEQ ID NO. 42) | TRBD2*01 |
| HPV16-E6 | KQRFHNIRGRWTGRC | DQB1*03:01/ | 24.73% | CDR3α sequence: | TRAV13-2*01, |
| | (SEQ ID NO. 6) | DRB1*15:01 | | CAETLGLDQGGKLIF | TRAJ23*01 |
| | | | | (SEQ ID NO. 43) | |
| | | | | CDR3β sequence: | TRBV20-1*03 |
| | | | | CSTAGETDTQYF | TRBJ2-3*01 |
| | | | | (SEQ ID NO. 44) | TRBD2*01 |
| | | | | CDR3α sequence: | TRAV17*01 |
| | | | | CATDEGTASKLTF | TRAJ49*01 |
| | | | | (SEQ ID NO. 45) | |
| | | | | CDR3β sequence: | TRBV29-1*01, |
| | | | | CSVEWTSGSGETQYF | TRBJ2-5*01, |
| | | | | (SEQ ID NO. 46) | TRBD2*02 |
| | | | | CDR3α sequence: | TRAV17*01, |
| | | | | CATDAGSDYKLSF | TRAJ20*01 |
| | | | | (SEQ ID NO. 47) | |
| | | | | CDR3β sequence: | TRBV28*01, |
| | | | | CASSLEPQYF | TRBJ2-3*01 |
| | | | | (SEQ ID NO. 48) | TRBD1*01 |
| | | | | CDR3α sequence: | TRAV17*01, |
| | | | | CATDEGTGNQFYF | TRAJ49*01 |
| | | | | (SEQ ID NO. 49) | |
| | | | | CDR3β sequence: | TRBV7-2*01, |
| | | | | CASSWDTGTETQYF | TRBJ2-5*01, |
| | | | | (SEQ ID NO. 50) | TRBD1*01 |
| HPV16-E4 | WPTTPPRPI | B*07:02 | 23.23% | CAFPSSGTYKYIF | TRAV24*01 |
| | (SEQ ID NO. 7) | | | (SEQ ID NO. 51) | TRAJ40*01 |
| | | | | CASTAGTDTQYF | TRBV27*01 |
| | | | | (SEQ ID NO. 52) | TRBJ2-3*01 |
| | | | | | TRBD1*01 |
| HPV16-E7 | TPTLHEYMLDLQPET | DRB1*03:01/ | 41.63% | CDR3α sequence: | TRAV4*01 |
| | TDLY | DQB1*02:01 | | CLVGPYFGGGSYQLTF | TRAJ28*01 |
| | (SEQ ID NO. 217) | | | (SEQ ID NO. 219) | |
| | | | | CDR3β sequence: | TRBV3-1*01 |
| | | | | CASSQGTGRGNTEAFF | TRBJ1-1*01 |
| | | | | (SEQ ID NO. 220) | TRBD1*01 |
| | | | | CDR3α sequence: | TRAV3*01 |
| | | | | (SEQ ID NO. 221) | TRAJ49*01 |
| | | | | CAVREKGTGNQFYF | |
| | | | | CDR3β sequence: | TRBV5-4*01 |
| | | | | CASSLPQGTPETQYF | TRBJ2-5*01 |
| | | | | (SEQ ID NO. 222) | TRBD1*01 |
| | | | | CDR3α sequence: | TRAV8-6*02 |
| | | | | CGLSWGDKTDKLIF | TRAJ34*01 |
| | | | | (SEQ ID NO. 223) | |
| | | | | CDR3β sequence: | TRBV14*01 |
| | | | | CASSPSLAGVVPGELFF | TRBJ2-2*01 |
| | | | | (SEQ ID NO. 224) | TRBD2*01 |
| HPV16-E5 | SAFRCFIVY | B*35:01 | 11.87% | CDR3α sequence: | TRAV38-1*01 |
| | (SEQ ID NO. 218) | | | CAFMKPDGSGNTGKLIF | TRAJ37*01 |
| | | | | (SEQ ID NO. 225) | |
| | | | | CDR3β sequence: | TRBV7-2*01 |
| | | | | CASSLGQGAVGTDTQYF | TRBJ2-3*01 |
| | | | | (SEQ ID NO. 226) | TRBD1*01 |

The α and β chains that comprise the T-cell receptors of the invention disclosed herein may be produced by recombinant methodologies and strategies known to those skilled in the art. See, for example, Wälchli et al. (2011) A Practical Approach to T-Cell Receptor Cloning and Expression. PLOS ONE 6(11): e27930, incorporated herein by reference in its entirety. Gene sequences that may be used in constructing the α and β chains of the TCRs of the invention are known to those of skill in the art and can be found in immunogenetics and immunoinformatics databases such as the International ImMunoGeneTics Information System® (IMGT®), referenced herein for exemplary purposes and without limitation. Such genes may be used as the framework for inserting the sequences provided herein for the TCRs of the invention. Those of skill in the relevant art, knowing that TCRs will be expressed at the cell surface of the immune cell (e.g., T cell, or precursor cell thereof) will understand that a signal peptide sequence (i.e., localization sequence) is generally included (e.g., at the amino-terminus). It is understood that, once a polypeptide containing a signal peptide is expressed at the cell surface, the signal peptide has generally been proteolytically removed during processing of the polypeptide in, for example, the endoplasmic reticulum and translocation to the cell surface. Thus, polypeptides, such as the TCRs disclosed herein, are generally expressed at the cell surface as a mature protein lacking the signal peptide, whereas the precursor form of the polypeptide includes the signal peptide. The signal peptide can be the naturally occurring signal peptide of the receptor, or alternatively can be derived from a different protein, or synthetic.

Preferably, the nucleotide sequence of the TCRs of the invention (e.g., the α- and β-chains) are cloned into vectors, e.g., as vector inserts. Said insert sequence may be codon optimized for expression in human tissues. In some such embodiments, the TCRs of the invention are fully human TCRs. The TCRs of the invention may be partially murinized (e.g., the amino acids of the constant regions of each TCR α and β chain may be replaced with those of mouse constant regions). Preferably, the vector inserts are designed such that the α- and β-chains of the TCR are synthesized from a single, contiguous open reading frame. Such vector inserts may comprise a contiguous open reading frame wherein the sequence encoding the α- and β-chains of the TCR are separated by a linker sequence, such as a linker comprising a self-cleaving 2A oligopeptide sequence that is in frame. In certain preferred embodiments, such self-cleaving linkers further comprise a Furin cleavage site. For example and without limitation, nucleotide sequences of TCR constructs contemplated herein, and their sequence features, are described in Tables 2-9.

TABLE 2
E5-NLD-TCR construct features
E5-NLD-TCR lentivirus construct (SEQ ID NO. 53)

| FEATURES | Location (nt) |
|---|---|
| Amp(R) | 7316 . . . 8176 |
| pUC ori promoter | 8377 . . . 8917 |
| RSV promoter | 7 . . . 235 |
| 3′LTR | 5921 . . . 6155 |
| Intron | 526 . . . 1690 |
| f1 origin | 6730 . . . 7185 |
| 5′ LTR | 236 . . . 416 |
| SV40 ori | 6358 . . . 6571 |
| WPRE seq | 5242 . . . 5838 |
| human EF1a promoter | 1933 . . . 3322 |
| Intron | 2361 . . . 3303 |
| Furin | 4308 . . . 4319 |
| SGSG linker (SEQ ID NO: 233) | 4320 . . . 4331 |
| F2A | 4332 . . . 4403 |
| Kozak seq | 3363 . . . 3371 |
| TCR beta | 3372 . . . 4307 |
| TCR apha | 4404 . . . 5234 |

TABLE 3
E6-HDI-TCR construct features
E6-HDI-TCR lentivirus construct (SEQ ID NO. 54)

| FEATURES | Location (nt) |
|---|---|
| Amp(R) | 7307 . . . 8167 |
| pUC ori promoter | 8368 . . . 8908 |
| RSV promoter | 7 . . . 235 |
| 3′LTR | 5912 . . . 6146 |
| Intron | 526 . . . 1690 |
| f1 origin | 6721 . . . 7176 |
| 5′ LTR | 236 . . . 416 |
| SV40 ori | 6349 . . . 6562 |
| WPRE seq | 5233 . . . 5829 |
| human EF1a promoter | 1933 . . . 2328 |
| Intron | 2361 . . . 3303 |
| Furin | 4323 . . . 4334 |
| SGSG linker (SEQ ID NO: 233) | 4335 . . . 4346 |
| F2A linker | 4347 . . . 4418 |
| Kozak seq | 3363 . . . 3371 |
| TCR beta | 3372 . . . 4322 |
| TCR apha | 4419 . . . 5225 |

TABLE 4
E2-TLQ-TCR construct features
E2-TLQ-TCR lentivirus construct (SEQ ID NO. 55)

| FEATURES | Location (nt) |
|---|---|
| Amp(R) | 7319 . . . 8179 |
| pUC ori promoter | 8380 . . . 8920 |
| RSV promoter | 7 . . . 235 |
| 3′LTR | 5924 . . . 6158 |
| Intron | 526 . . . 1690 |
| f1 origin | 6733 . . . 7188 |
| 5′ LTR | 236 . . . 416 |
| SV40 ori | 6361 . . . 6574 |
| WPRE seq | 5245 . . . 5841 |
| human EF1a promoter | 1933 . . . 3322 |
| Intron | 2361 . . . 3303 |
| Furin | 4314 . . . 4325 |
| SGSG linker (SEQ ID NO: 233) | 4326 . . . 4337 |
| F2A linker | 4338 . . . 4409 |
| Kozak seq | 3363 . . . 3371 |
| TCR beta | 3372 . . . 4313 |
| TCR alpha | 4410 . . . 5237 |

TABLE 5
E6-TIH-TCR construct features
E6-TIH-TCR lentivirus construct (SEQ ID NO. 56)

| FEATURES | Location (nt) |
|---|---|
| Amp(R) | 7286 . . . 8146 |
| pUC ori promoter | 8347 . . . 8887 |
| RSV promoter | 7 . . . 235 |
| 3′LTR | 5891 . . . 6125 |
| Intron | 526 . . . 1690 |
| f1 origin | 6700 . . . 7155 |
| 5′ LTR | 236 . . . 416 |
| SV40 ori | 6328 . . . 6541 |
| WPRE seq | 5212 . . . 5808 |
| human EF1a promoter | 1933 . . . 3322 |
| Intron | 2361 . . . 3303 |
| Furin | 4302 . . . 4313 |
| SGSG linker (SEQ ID NO: 233) | 4314 . . . 4325 |
| F2A linker | 4326 . . . 4397 |
| Kozak seq | 3363 . . . 3371 |
| TCR beta | 3372 . . . 4301 |
| TCR apha | 4398 . . . 5204 |

TABLE 6
E6-AFR-TCR construct features
E6-AFR-TCR lentivirus construct (SEQ ID NO. 57)

| FEATURES | Location (nt) |
|---|---|
| Amp(R) | 7307 . . . 8167 |
| pUC ori promoter | 8368 . . . 8908 |
| RSV promoter | 7 . . . 235 |
| 3′LTR | 5912 . . . 6146 |
| Intron | 526 . . . 1690 |
| f1 origin | 6721 . . . 7176 |
| 5′ LTR | 236 . . . 416 |
| SV40 ori | 6349 . . . 6562 |
| WPRE seq | 5233 . . . 5829 |
| human EF1a promoter | 1933 . . . 3322 |
| Intron | 2361 . . . 3303 |
| Furin | 4305 . . . 4316 |
| SGSG linker (SEQ ID NO: 233) | 4317 . . . 4328 |
| F2A linker | 4329 . . . 4400 |
| Kozak seq | 3363 . . . 3371 |
| TCR beta | 3372 . . . 4304 |
| TCR apha | 4401 . . . 5225 |

TABLE 7
E6-KQR-TCR construct features
E6-KQR-TCR lentivirus construct (SEQ ID NO. 58)

| FEATURES | Location (nt) |
|---|---|
| Amp(R) | 7328 . . . 8188 |
| pUC ori promoter | 8389 . . . 8929 |
| RSV promoter | 7 . . . 235 |
| 3′LTR | 5933 . . . 6167 |
| Intron | 526 . . . 1690 |
| f1 origin | 6742 . . . 7197 |
| 5′ LTR | 236 . . . 416 |
| SV40 ori | 6370 . . . 6583 |
| WPRE seq | 5254 . . . 5850 |
| human EF1a promoter | 1933 . . . 3322 |
| Intron | 2361 . . . 3303 |
| Furin | 4326 . . . 4337 |
| SGSG linker (SEQ ID NO: 233) | 4338 . . . 4349 |
| F2A linker | 4350 . . . 4421 |
| Kozak seq | 3363 . . . 3371 |
| TCR beta | 3372 . . . 4325 |
| TCR alpha | 4422 . . . 5246 |

TABLE 8
E7-TPT-TCR construct features
E7-TPT-TCR lentivirus construct (SEQ ID NO. 227)

| FEATURES | Location (nt) |
|---|---|
| Amp(R) | 7295 . . . 8155 |
| pUC ori promoter | 8356 . . . 8896 |
| RSV promoter | 7 . . . 235 |
| 3′LTR | 5900 . . . 6134 |
| Intron | 526 . . . 1690 |
| f1 origin | 6709 . . . 7164 |
| 5′ LTR | 236 . . . 416 |
| SV40 ori | 6337 . . . 6550 |
| WPRE seq | 5221 . . . 5817 |
| human EF1a promoter | 1933 . . . 3322 |
| Intron | 2361 . . . 3303 |
| Furin | 4305 . . . 4316 |
| SGSG linker (SEQ ID NO: 233) | 4317 . . . 4328 |
| F2A linker | 4329 . . . 4400 |
| Kozak seq | 3363 . . . 3371 |
| TCR beta | 3372 . . . 4304 |
| TCR alpha | 4401 . . . 5213 |

TABLE 9
E5-SAF-TCR construct features
E5-SAF-TCR lentivirus construct (SEQ ID NO. 228)

| FEATURES | Location (nt) |
|---|---|
| Amp(R) | 7340 . . . 8200 |
| PUC ori promoter | 8401 . . . 8941 |
| RSV promoter | 7 . . . 235 |
| 3′LTR | 5945 . . . 6179 |
| Intron | 526 . . . 1690 |
| f1 origin | 6754 . . . 7209 |
| 5′ LTR | 236 . . . 416 |
| SV40 ori | 6382 . . . 6595 |
| WPRE seq | 5266 . . . 5862 |
| human EF1a promoter | 1933 . . . 3322 |
| Intron | 2361 . . . 3303 |
| Furin | 4326 . . . 4337 |
| SGSG linker (SEQ ID NO: 233) | 4338 . . . 4349 |
| F2A linker | 4350 . . . 4421 |
| Kozak seq | 3363 .. . 3371 |
| TCR beta | 3372 . . . 4325 |
| TCR alpha | 4422 . . . 5258 |

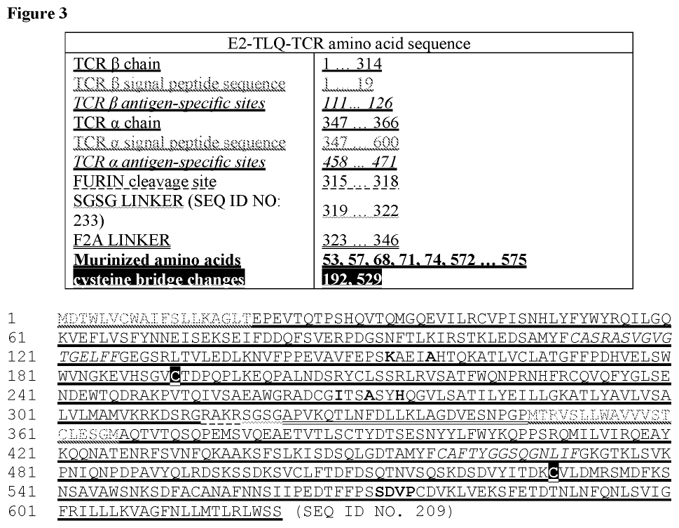

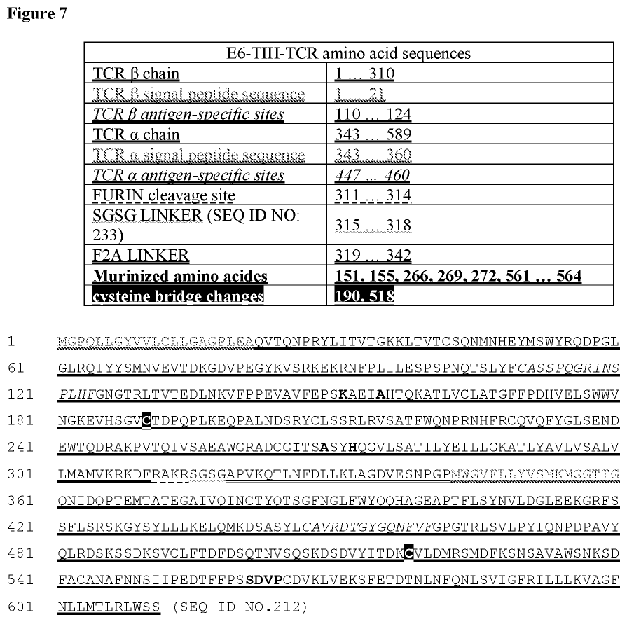

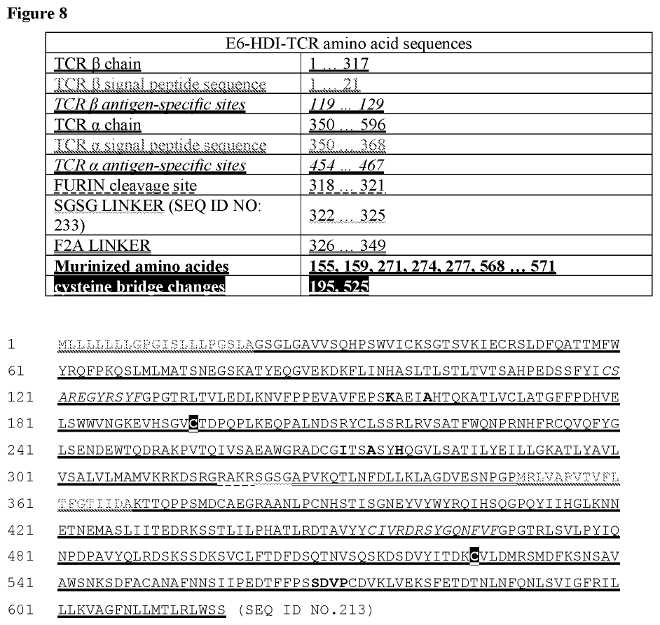

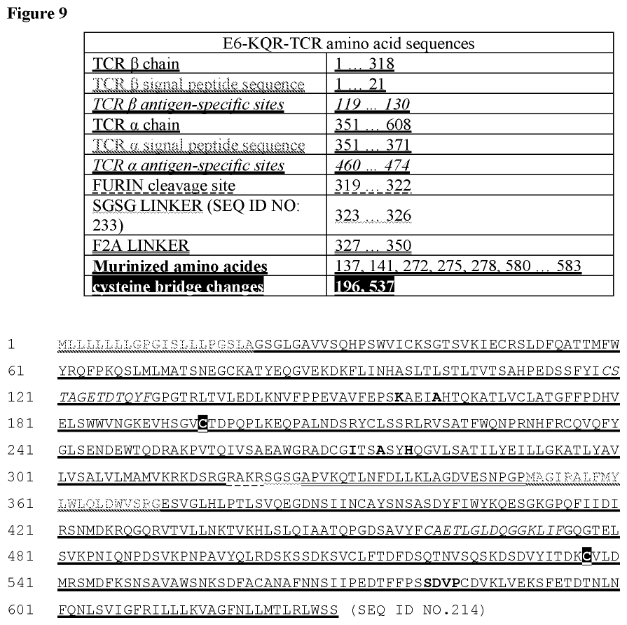

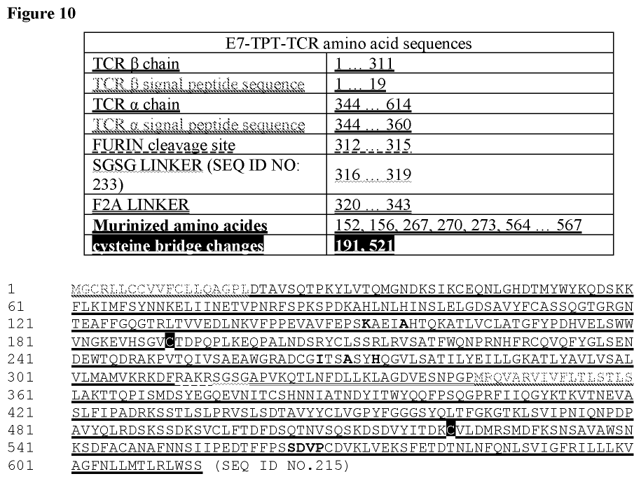

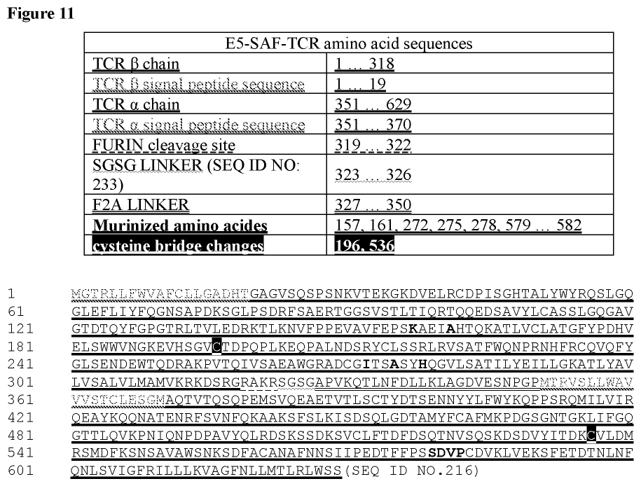

Likewise, exemplary TCRs (e.g., TCR α/β peptide chains) encoded by the constructs contemplated herein are described in in FIGS. 3, 4, and 6-11. In some embodiments, the TCRs expressed at the cell surface comprise at least one TCR chain comprising an amino acid sequence set forth in Table 10. In preferred embodiments, such TCRs comprise a TCRα chain and TCRβ chain, each respectively comprising an amino acid sequence set forth in Table 10. For example, the TCR may comprise a TCRβ chain having the amino acid sequence set forth in SEQ ID NO. 59 and a TCRα chain having the amino acid sequence set forth in SEQ ID NO. 60; or a TCRβ chain having the amino acid sequence set forth in SEQ ID NO. 61 and a TCRα chain having the amino acid sequence set forth in SEQ ID NO. 62

TABLE 10
Engineered TCR α and B chains

| TCR Construct | α/ chain | Amino Acid Sequence |
|---|---|---|
| E2-TLQ-TCR | TCR β | EPEVTQTPSHQVTQMGQEVILRCVPISNHLYFYWYRQILGQ |
| | | KVEFLVSFYNNEISEKSEIFDDQFSVERPDGSNFTLKIRSTKL |
| | | EDSAMYFCASRASVGVGTGELFFGEGSRLTVLEDLKNVFPP |
| | | EVAVFEPSKAEIAHTQKATLVCLATGFFPDHVELSWWVNG |
| | | KEVHSGVCTDPQPLKEQPALNDSRYCLSSRLRVSATFWQNP |
| | | RNHFRCQVQFYGLSENDEWTQDRAKPVTQIVSAEAWGRAD |
| | | CGITSASYHQGVLSATILYEILLGKATLYAVLVSALVLMAM |
| | | VKRKDSRG (SEQ ID NO. 59) |
| | TCR α | AQTVTQSQPEMSVQEAETVTLSCTYDTSESNYYLFWYKQPP |
| | | SRQMILVIRQEAYKQQNATENRFSVNFQKAAKSFSLKISDSQ |
| | | LGDTAMYFCAFTYGGSQGNLIFGKGTKLSVKPNIQNPDPAV |
| | | YQLRDSKSSDKSVCLFTDFDSQTNVSQSKDSDVYITDKCVL |
| | | DMRSMDFKSNSAVAWSNKSDFACANAFNNSIIPEDTFFPSS |
| | | DVPCDVKLVEKSFETDTNLNFQNLSVIGFRILLLKVAGFNLL |
| | | MTLRLWSS (SEQ ID NO. 60) |
| E5-NLD-TCR | TCR β | GVSQSPRYKVTKRGQDVALRCDPISGHVSLYWYRQALGQG |
| | | PEFLTYFNYEAQQDKSGLPNDRFSAERPEGSISTLTIQRTEQR |
| | | DSAMYRCASSPELAGPQETQYFGPGTRLLVLEDLNKVFPPE |
| | | VAVFEPSKAEIAHTQKATLVCLATGFFPDHVELSWWVNGK |
| | | EVHSGVCTDPQPLKEQPALNDSRYCLSSRLRVSATFWQNPR |
| | | NHFRCQVQFYGLSENDEWTQDRAKPVTQIVSAEAWGRADC |
| | | GITSASYHQGVLSATILYEILLGKATLYAVLVSALVLMAMV |
| | | KRKDF (SEQ ID NO. 61) |
| | TCR α | AQKVTQAQTEISVVEKEDVTLDCVYETRDTTYYLFWYKQP |
| | | PSGELVFLIRRNSFDEQNEISGRYSWNFQKSTSSFNFTITASQ |
| | | VVDSAVYFCALSEGGGSQGNLIFGKGTKLSVKPNIQNPDPA |
| | | VYQLRDSKSSDKSVCLFTDFDSQTNVSQSKDSDVYITDKCV |
| | | LDMRSMDFKSNSAVAWSNKSDFACANAFNNSIIPEDTFFPSS |
| | | DVPCDVKLVEKSFETDTNLNFQNLSVIGFRILLLKVAGFNLL |
| | | MTLRLWSS (SEQ ID NO. 62) |
| E6-AFR-TCR | TCR β | MTIRLLCYMGFYFLGAGLMEADIYQTPRYLVIGTGKKITLEC |
| | | SQTMGHDKMYWYQQDPGMELHLIHYSYGVNSTEKGDLSS |
| | | ESTVSRIRTEHFPLTLESARPSHTSQYLCASSGPGQGHNQPQ |
| | | HFGDGTRLSILEDLNKVFPPEVAVFEPSKAEIAHTQKATLVC |
| | | LATGFFPDHVELSWWVNGKEVHSGVCTDPQPLKEQPALND |
| | | SRYCLSSRLRVSATFWQNPRNHFRCQVQFYGLSENDEWTQ |
| | | DRAKPVTQIVSAEAWGRADCGITSASYHQGVLSATILYEILL |
| | | GKATLYAVLVSALVLMAMVKRKDF (SEQ ID NO. 63) |
| | TCR α | QKVTQAQTEISVVEKEDVTLDCVYETRDTTYYLFWYKQPPS |
| | | GELVFLIRRNSFDEQNEISGRYSWNFQKSTSSFNFTITASQVV |
| | | DSAVYFCALGSSGTYKYIFGTGTRLKVLANIQNPDPAVYQL |
| | | RDSKSSDKSVCLFTDFDSQTNVSQSKDSDVYITDKCVLDMR |
| | | SMDFKSNSAVAWSNKSDFACANAFNNSIIPEDTFFPSSDVPC |
| | | DVKLVEKSFETDTNLNFQNLSVIGFRILLLKVAGFNLLMTLR |
| | | LWSS (SEQ ID NO. 64) |
| E6-TIH-TCR | TCR β | QVTQNPRYLITVTGKKLTVTCSQNMNHEYMSWYRQDPGL |
| | | GLRQIYYSMNVEVTDKGDVPEGYKVSRKEKRNFPLILESPSP |
| | | NQTSLYFCASSPQGRINSPLHFGNGTRLTVTEDLNKVFPPEV |
| | | AVFEPSKAEIAHTQKATLVCLATGFFPDHVELSWWVNGKE |
| | | VHSGVCTDPQPLKEQPALNDSRYCLSSRLRVSATFWQNPRN |
| | | HFRCQVQFYGLSENDEWTQDRAKPVTQIVSAEAWGRADCG |
| | | ITSASYHQGVLSATILYEILLGKATLYAVLVSALVLMAMVK |
| | | RKDF (SEQ ID NO. 65) |
| | TCR α | QNIDQPTEMTATEGAIVQINCTYQTSGFNGLFWYQQHAGEA |
| | | PTFLSYNVLDGLEEKGRFSSFLSRSKGYSYLLLKELQMKDS |
| | | ASYLCAVRDTGYGQNFVFGPGTRLSVLPYIQNPDPAVYQLR |
| | | DSKSSDKSVCLFTDFDSQTNVSQSKDSDVYITDKCVLDMRS |
| | | MDFKSNSAVAWSNKSDFACANAFNNSIIPEDTFFPSSDVPCD |
| | | VKLVEKSFETDTNLNFQNLSVIGFRILLLKVAGFNLLMTLRL |
| | | WSS (SEQ ID NO. 66) |
| E6-HDI-TCR | TCR β | GSGLGAVVSQHPSWVICKSGTSVKIECRSLDFQATTMFWYR |
| | | QFPKQSLMLMATSNEGSKATYEQGVEKDKFLINHASLTLST |
| | | LTVTSAHPEDSSFYICSAREGYRSYFGPGTRLTVLEDLKNVF |
| | | PPEVAVFEPSKAEIAHTQKATLVCLATGFFPDHVELSWWVN |
| | | GKEVHSGVCTDPQPLKEQPALNDSRYCLSSRLRVSATFWQN |
| | | PRNHFRCQVQFYGLSENDEWTQDRAKPVTQIVSAEAWGRA |
| | | DCGITSASYHQGVLSATILYEILLGKATLYAVLVSALVLMA |
| | | MVKRKDSRG (SEQ ID NO. 67) |
| | TCR α | KTTQPPSMDCAEGRAANLPCNHSTISGNEYVYWYRQIHSQG |
| | | PQYIIHGLKNNETNEMASLIITEDRKSSTLILPHATLRDTAVY |
| | | YCIVRDRSYGQNFVFGPGTRLSVLPYIQNPDPAVYQLRDSKS |
| | | SDKSVCLFTDFDSQTNVSQSKDSDVYITDKCVLDMRSMDFK |
| | | SNSAVAWSNKSDFACANAFNNSIIPEDTFFPSSDVPCDVKLV |
| | | EKSFETDTNLNFQNLSVIGFRILLLKVAGFNLLMTLRLWSS |
| | | (SEQ ID NO. 68) |
| E6-KQR-TCR | TCR β | GSGLGAVVSQHPSWVICKSGTSVKIECRSLDFQATTMFWYR |
| | | QFPKQSLMLMATSNEGCKATYEQGVEKDKFLINHASLTLST |
| | | LTVTSAHPEDSSFYICSTAGETDTQYFGPGTRLTVLEDLKNV |
| | | FPPEVAVFEPSKAEIAHTQKATLVCLATGFFPDHVELSWWV |
| | | NGKEVHSGVCTDPQPLKEQPALNDSRYCLSSRLRVSATFWQ |
| | | NPRNHFRCQVQFYGLSENDEWTQDRAKPVTQIVSAEAWGR |
| | | ADCGITSASYHQGVLSATILYEILLGKATLYAVLVSALVLM |
| | | AMVKRKDSRG (SEQ ID NO. 69) |
| | TCR α | ESVGLHLPTLSVQEGDNSIINCAYSNSASDYFIWYKQESGKG |
| | | PQFIIDIRSNMDKRQGQRVTVLLNKTVKHLSLQIAATQPGDS |
| | | AVYFCAETLGLDQGGKLIFGQGTELSVKPNIQNPDSVKPNP |
| | | AVYQLRDSKSSDKSVCLFTDFDSQTNVSQSKDSDVYITDKC |
| | | VLDMRSMDFKSNSAVAWSNKSDFACANAFNNSIIPEDTFFP |
| | | SSDVPCDVKLVEKSFETDTNLNFQNLSVIGFRILLLKVAGFN |
| | | LLMTLRLWSS (SEQ ID NO. 70) |
| E7-TPT-TCR | TCR β | DTAVSQTPKYLVTQMGNDKSIKCEQNLGHDTMYWYKQDS |
| | | KKFLKIMFSYNNKELIINETVPNRFSPKSPDKAHLNLHINSLE |
| | | LGDSAVYFCASSQGTGRGNTEAFFGQGTRLTVVEDLNKVFP |
| | | PEVAVFEPSKAEIAHTQKATLVCLATGFYPDHVELSWWVN |
| | | GKEVHSGVCTDPQPLKEQPALNDSRYCLSSRLRVSATFWQN |
| | | PRNHFRCQVQFYGLSENDEWTQDRAKPVTQIVSAEAWGRA |
| | | DCGITSASYHQGVLSATILYEILLGKATLYAVLVSALVLMA |
| | | MVKRKDF (SEQ ID NO. 229) |
| | TCR α | LAKTTQPISMDSYEGQEVNITCSHNNIATNDYITWYQQFPSQ |
| | | GPRFIIQGYKTKVTNEVASLFIPADRKSSTLSLPRVSLSDTAV |
| | | YYCLVGPYFGGGSYQLTFGKGTKLSVIPNIQNPDPAVYQLR |
| | | DSKSSDKSVCLFTDFDSQTNVSQSKDSDVYITDKCVLDMRS |
| | | MDFKSNSAVAWSNKSDFACANAFNNSIIPEDTFFPSSDVPCD |
| | | VKLVEKSFETDTNLNFQNLSVIGFRILLLKVAGFNLLMTLRL |
| | | WSS (SEQ ID NO. 230) |
| E5-SAF-TCR | TCR β | GAGVSQSPSNKVTEKGKDVELRCDPISGHTALYWYRQSLG |
| | | QGLEFLIYFQGNSAPDKSGLPSDRFSAERTGGSVSTLTIQRTQ |
| | | QEDSAVYLCASSLGQGAVGTDTQYFGPGTRLTVLEDRKTL |
| | | KNVFPPEVAVFEPSKAEIAHTQKATLVCLATGFYPDHVELS |
| | | WWVNGKEVHSGVCTDPQPLKEQPALNDSRYCLSSRLRVSA |
| | | TFWQNPRNHFRCQVQFYGLSENDEWTQDRAKPVTQIVSAE |
| | | AWGRADCGITSASYHQGVLSATILYEILLGKATLYAVLVSA |
| | | LVLMAMVKRKDSRG (SEQ ID NO. 231) |
| | TCR α | AQTVTQSQPEMSVQEAETVTLSCTYDTSENNYYLFWYKQP |
| | | PSRQMILVIRQEAYKQQNATENRFSVNFQKAAKSFSLKISDS |
| | | QLGDTAMYFCAFMKPDGSGNTGKLIFGQGTTLQVKPNIQNP |
| | | DPAVYQLRDSKSSDKSVCLFTDFDSQTNVSQSKDSDVYITD |
| | | KCVLDMRSMDFKSNSAVAWSNKSDFACANAFNNSIIPEDTF |
| | | FPSSDVPCDVKLVEKSFETDTNLNFQNLSVIGFRILLLKVAG |
| | | FNLLMTLRLWSS (SEQ ID NO. 232) |

Similarly, embodiments of the invention include immune effector cells (e.g., T cells) which have been transduced with such constructs so as to express the engineered TCRs.

In some embodiments, the TCRs disclosed herein specifically bind an epitope listed in Table 11.

TABLE 11
HPV-specific epitopes

| | | HLA |
|---|---|---|
| Antigen | Epitope | restriction |
| HPV16-E1 | YLHNRLVVF | B*08:01 |
| | (SEQ ID NO. 8) | |
| HPV16-E1 | ALDGNLVSMDV | A*02:01 |
| | (SEQ ID NO. 9) | |
| HPV18-E6 | TVLELTEVFEF | A*02:01 |
| | (SEQ ID NO. 10) | |
| HPV18-E5 | SPATAFTVY | B*35:01 |
| | (SEQ ID NO. 11) | |
| HPV16-E7 | VQSTHVDIRTLEDLLMGTL | DQB1*03:01 |
| | (SEQ ID NO. 12) | |

In certain embodiments, the TCR (e.g., the immune effector cell expressing the engineered TCR) can be applied and/or administered using a plurality of strategies known in the art. For example (and without limitation), TRUCKs (T cells redirected for universal cytokine killing) co-express a modified (e.g., artificial/recombinant/exogenous) TCR and an antitumor cytokine. Cytokine expression may be constitutive or induced by T cell activation. Targeted by TCR-specificity, localized production of pro-inflammatory cytokines recruits endogenous immune cells to tumor sites and may potentiate an antitumor response.

Alternatively, allogeneic TCR-T cells may be engineered by means known in the art to no longer express endogenous T cell receptor (TCR) and/or major histocompatibility complex (MHC) molecules, thereby improving expression and/or function of the exogenous TCR and/or preventing or reducing graft-versus-host disease (GVHD) or rejection, respectively.

A TCR-T cell may be engineered to co-express a TCR and a chemokine receptor, which binds to a tumor ligand, thereby enhancing tumor homing.

TCR-T cells engineered to be resistant to immunosuppression may be genetically modified to no longer express various immune checkpoint molecules (e.g., cytotoxic T lymphocyte-associated antigen 4 (CTLA4) or programmed cell death protein 1 (PD-1)). Exemplary “Knockdown” and “Knockout” techniques include, but are not limited to, RNA interference (RNAi) (e.g., asRNA, miRNA, shRNA, siRNA, etc.) and CRISPR interference (CRISPRi) (e.g., CRISPR-Cas9). In certain embodiments, TCR-T cells are engineered to express a dominant-negative form of a checkpoint molecule. In some such embodiments, the extracellular ligand-binding domain (i.e., ectodomain) of the immune checkpoint molecule is fused to a transmembrane membrane in order to compete for ligand binding. For example, the extracellular ligand-binding domain of PD-1 may be fused to a CD8 transmembrane domain, thus competing for PD-1 ligand from the target cell. In some embodiments, TCR-T cells are engineered to express an immune checkpoint switch receptor to exploit the inhibitory immune checkpoint ligand present on a target cell. In such embodiments, the extracellular ligand-binding domain of the immune checkpoint molecule is fused to a signaling, stimulatory, and/or co-stimulatory domain. For example, the extracellular ligand-binding domain of PD-1 may be fused to a CD28 domain, thus providing CD28 co-stimulation while blocking PD-1 signaling. In further embodiments, the TCR-T cells may be administered with an aptamer or a monoclonal antibody that blocks immune checkpoint signaling. In some such embodiments, the TCR-T cell (e.g., TCR-T cell therapy) is combined with a PD-1 blockade method, such as administration with PD-1/PD-L1 antagonistic aptamers or anti-PD-1/PD-L1 antibodies. In preferred embodiments, the TCR-T cells and PD-1 pathway-blocking antibodies are administered conjointly. In further embodiments, the TCR-T cells are engineered to express or express and secrete an immune checkpoint-blocking antibody, such as anti-PD-1 or anti-PD-L1, or fragments thereof. In yet further embodiments, the TCR-T cells are administered with a vector (e.g., an engineered virus) that expresses an immune checkpoint-blocking molecule described herein.

A self-destruct TCR-T cell may be designed using inducible apoptosis of the T cell, e.g., by ganciclovir binding to thymidine kinase in gene-modified lymphocytes or by activation of human caspase 9 by a small-molecule dimerizer.

A marked TCR-T cell expresses a modified TCR plus a tumor epitope to which an existing monoclonal antibody agent binds. In the setting of intolerable adverse effects, administration of the monoclonal antibody clears the TCR-T cells and alleviates symptoms with no additional off-tumor effects.

A bi-specific TCR-T cell may further express another TCR or a chimeric antigen receptor (CAR) with different antigen/ligand binding targets relative to the first modified TCR, such as other cancer-associated antigens, including tumor antigens.

Tumor antigens include proteins that are produced by tumor cells that elicit an immune response; particularly T cell mediated immune responses. The additional antigen binding domain can be an antibody or a natural ligand of the tumor antigen. The selection of the additional antigen-binding domain will depend on the particular type of cancer to be treated. Tumor antigens are well known in the art and include, for example, a glioma-associated antigen, carcinoembryonic antigen (CEA), EGFRvIII, IL-11Ra, IL-13Ra, EGFR, FAP, B7H3, Kit, CA LX, CS-1, MUC1, BCMA, bcr-abl, HER2, β-human chorionic gonadotropin, alphafetoprotein (AFP), ALK, alternate and/or specific CD19 epitopes, TIM3, cyclin B1, lectin-reactive AFP, Fos-related antigen 1, ADRB3, thyroglobulin, EphA2, RAGE-1, RU1, RU2, SSX2, AKAP-4, LCK, OY-TES1, PAX5, SART3, CLL-1, fucosyl GM1, GloboH, MN-CA IX, EPCAM, EVT6-AML, TGS5, human telomerase reverse transcriptase, plysialic acid, PLAC1, RU1, RU2 (AS), intestinal carboxyl esterase, lewisY, sLe, LY6K, HSP70, HSP27, mut hsp70-2, M-CSF, MYCN, RhoC, TRP-2, CYPIBI, BORIS, prostase, prostate-specific antigen (PSA), PAX3, PAP, NY-ESO-1, LAGE-1a, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6,E7, sperm protein 17, SSEA-4, tyrosinase, TARP, WT1, prostate-carcinoma tumor antigen-1 (PCTA-1), ML-IAP, MAGE, MAGE-A1, MAGE-A2, MAGE-C1, MAGE-C2, Annexin-A2, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, EphrinB2, CD20, CD22, CD24, CD30, TIM3, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. In certain preferred embodiments, the tumor antigen is selected from folate receptor (FRa), mesothelin, EGFRvIII, IL-13Ra, CD123, CD19, TIM3, BCMA, GD2, CLL-1, CA-IX, MUC1, HER2, and any combination thereof.

Further non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include SCCA, GP73, FC-GP73, TSP-180, MAGE-4, MAGE-5, MAGE-6, RAGE, NY-ESO, pl85erbB2, pl80erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG1 6, TA-90\Mac-2 binding protein\cyclophilin C-associated protein, TAAL6, TAG72, TLP, TPS, GPC3, MUC16, TAG-72, LMP1, EBMA-1, BARF-1, CS1, CD319, HER1, B7H6, L1CAM, IL6, and MET.

Generally, with respect to the methods disclosed herein, almost any strategy applied to CAR-T cells may be applied to the TCR-T cells disclosed herein.

Nucleic Acids and Vectors

Also disclosed are polynucleotides and polynucleotide vectors encoding the disclosed HPV antigen-specific TCRs that allow expression of the HPV antigen-specific TCRs in the disclosed immune effector cells. In some embodiments, polynucleotides and polynucleotide vectors disclosed herein comprise any one of the nucleic acid sequences set forth in Tables 2-9. In certain aspects of the invention, provided herein are isolated nucleic acids comprising a nucleotide sequence encoding a peptide (or peptides) comprising a TCRα and/or TCRβ chain. In some embodiments, the peptides encoded by the nucleic acids disclosed herein comprise an amino acid sequence selected from any one of SEQ ID NOs. 13-52, 59-70, 209-216, or fragments thereof.

Nucleic acid sequences encoding the disclosed TCRs, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Expression of nucleic acids encoding TCRs is typically achieved by operably linking a nucleic acid encoding the TCR polypeptide to a promoter, and incorporating the construct into an expression vector. Typical cloning and/or expression vectors contain transcription and translation terminators, initiation sequences, and promoters useful for regulation of the expression of the desired nucleic acid sequence.

The nucleic acid sequences encoding the α and β chains of the TCRs of the present invention may be placed in a single expression vector by methods known in the art. For example and without limitation, the nucleic acid sequences encoding the TCRs described herein may comprise a nucleic acid sequence encoding a ribosomal skip sequence such as a sequence encoding a 2A peptide. 2A peptides, which were identified in the Aphthovirus subgroup of picornaviruses, causes a ribosomal “skip” from one codon to the next without the formation of a peptide bond between the two amino acids encoded by the codons. Thus, two polypeptides can be synthesized from a single, contiguous open reading frame within an mRNA when the polypeptides are separated by a 2A oligopeptide sequence that is in frame (e.g., when the alpha and beta chains of the TCR are separated by a 2A oligopeptide sequence). Such ribosomal “skip” or “self-cleaving” mechanisms or are well known in the art and are known to be used by several vectors for the expression of several proteins encoded by a single messenger RNA. For example, the 2A oligopeptide sequence may be used with a furin cleavage recognition site. Preferably, the furin recognition site is upstream from the 2A oligopeptide sequence. Most preferably, the furin recognition site sequence and the 2A oligopeptide sequence are separated by a GSG linker. Such bicistronic lentiviral vectors combining a furin cleavage site, and an amino acid spacer followed by a 2A ribosomal skip peptide are known in the art. See, for example, Yang et al. (2008) Development of optimal bicistronic lentiviral vectors facilitates high-level TCR gene expression and robust tumor cell recognition, Gene Therapy volume 15, pages 1411-1423, incorporated herein by reference in its entirety. The resultant peptide upstream from the self-cleaving furin-spacer-2A site may retain the furin recognition sequence at its carboxy-terminus (e.g., the FURIN cleavage site sequence indicated in FIGS. 3, 4, and 6-11). Likewise, in the absence of any post-translational modifications, such as the removal of a signal peptide sequence, the resultant peptide downstream from the self-cleaving furin-spacer-2A site may retain amino acids at its amino-terminus (e.g., the terminal proline of the F2A linker sequence indicated in FIGS. 3, 4, and 6-11). Alternatively the α and β chains may each be placed in a separate expression vector.

The disclosed nucleic acids can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. In some embodiments, the polynucleotide vectors are lentiviral or retroviral vectors.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. Preferably, gene transfer is into mammalian cells (e.g., PBMCs).

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α; EF1α). However, other constitutive promoter sequences may also be used, including, but not limited to the simian virus 40 (SV40) early promoter, MND (myeloproliferative sarcoma virus) promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. The promoter can alternatively be an inducible promoter. Examples of inducible promoters include, but are not limited to a metallothionine promoter, a glucocorticoid promoter, a progesterone promoter, and a tetracycline promoter.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another.

In order to assess the expression of a TCR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc, (Birmingham, Ala.).

Immune Cells

The α and β chains of the TCRs of the invention disclosed herein may be expressed independently in different host cells or in the same host cell. In preferred embodiments, the α and β chains are introduced into the same host cell to allow for formation of a functional T-cell receptor. Most preferably, the host cells engineered to express all or part of the disclosed TCRs of the invention include immune cells (e.g., immune effector cells). Such cells may be obtained from the subject (i.e., the donor) to be treated (i.e., autologous cells). However, in some embodiments, immune cell lines or donor cells other than the subject's own cells (i.e., allogeneic cells) are used. Immune effector cells can be obtained from a number of sources, including peripheral blood mononuclear cells (PBMCs), bone marrow, lymph node tissue, cord blood, thymus tissue, tissue from a site of infection, ascites, pleural effusion, spleen tissue, and tumors. Immune effector cells can be obtained from blood collected from a subject and/or donor using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dendritic cells, mast cells, neutrophils, basophils, eosinophils, or any combinations thereof. For example, the immune effector cells can comprise T lymphocytes, preferably cytotoxic T lymphocytes (CTLs).

T cells or T lymphocytes can be distinguished from other lymphocytes, such as B cells and natural killer cells (NK cells), by the presence of a T cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4⁺ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (Tc cells, or CTLs) destroy virally infected cells and tumor cells, and are also implicated in transplant rejection. These cells are also known as CD8⁺ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8⁺ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4⁺ cells. In other embodiments, the T cells are enriched for one or more subsets based on cell surface expression. For example, in some cases, the T comprise are cytotoxic CD8⁺ T lymphocytes. In some embodiments, the T cells comprise γδ T cells, which possess a distinct T cell receptor (TCR) having one γ chain and one S chain instead of α and β chains.

Natural-killer (NK) cells are CD56*CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects. Although NK cells have a well-known role as killers of cancer cells, and NK cell impairment has been extensively documented as crucial for progression of Multiple myeloma (MM) (Godfrey J, et al. Leuk Lymphoma 2012 53:1666-1676; Fauriat C, et al. Leukemia 2006 20:732-733), the means by which one might enhance NK cell-mediated anti-MM activity has been largely unexplored prior to the disclosed TCRs.

While innate immune responses play an important role in controlling initial HPV infection, long-term protection is dependent on adaptive immune responses including humoral and cell-mediated immunity. In immunocompetent individuals, the majority of HPV infections are cleared within 2 years of initial infection. Infiltration of CD4⁺ and CD8⁺ T cells is frequently observed in spontaneously regressing lesions. Though the HPV vaccines act prophylactically and are based on the L1 protein, this viral antigen is not relevant for the treatment of HPV-associated diseases. L1 protein is only expressed in the late stages of HPV replication especially in terminally differentiated keratinocytes. In contrast, other proteins associated with the HPV replicative cycle, i.e., E1, E2, E4, E5, E6, and E7, may provide important targets for immunotherapeutic strategies. This is primarily due to the fact that the expression of all these proteins are retained through multiple stages of infection. While much of the emphasis on the design of immunotherapeutic strategies has focused on E6 and E7 antigens, it is important to appreciate that other early proteins are implicated in HPV replication and thus the expression of these proteins is retained throughout multiple stages of infection. This highlights the importance of these proteins as potential targets for immunotherapy aimed at eliminating persistently HPV-infected cells regardless of the stage of pathogenesis. Indeed, previous studies using animal models (canine and rabbit) have shown that immunization with a DNA vaccine encoding codon-optimised E1 or E2 genes results in complete regression of papillomas. The primary mode of protection in these animal models is mediated through the induction of an effective T cell response to E1 and E2 antigens. Further clinical studies using a modified vaccinia Ankara vector encoding E2 in human subjects with HPV-induced cervical lesions (C1N1 to C1N3) demonstrated complete elimination of cervical lesions to regression from C1N3 to C1N1 and significant reduction in HPV viral load. Here again, the induction of E2-specific T cell immunity correlated strongly with clinical response. Development of anti-vector antibodies resulted in a poor response to booster immunization and some patients showed recurrence of lesions after the completion of the study. Moreover, this therapy required direct injection of the vector into uterine tissue to be effective, thus limiting its wider use in the general population.

Thus, provided herein are methods for prophylaxis and treatment of HPV-associated diseases and cancers by the adoptive transfer of autologous or allogeneic HPV-specific, TCR-expressing cells (e.g., TCR-T cells described herein). Such methods may include the generation of and/or the use of peptide-specific T cells (e.g., CTLs, CD8⁺ T cells, and/or CD4⁺ T cells). The generation of peptide-specific T cells is known in the art and may include, for example, incubating a sample comprising T cells (e.g., a PBMC sample, an enriched sample, or a sample of isolated T cells) with antigenic peptides (i.e., peptides comprising T cell epitopes) or with antigen-presenting cells (APCs) that present one or more of such T cell epitopes (e.g., APCs that present a peptide comprising a CTL epitope on a class I MHC complex), thereby inducing the sensitization (e.g., activation and proliferation) of peptide-specific T cells. In some embodiments, the antigenic peptides comprise a sequence of any viral protein (i.e., antigen). For example, and without limitation, the immune cell (e.g., CTL) is sensitized to a viral antigen from any one of human papilloma virus (HPV), Epstein-Barr virus (EBV), cytomegalovirus (CMV), B.K. virus (BKV), John Cunningham virus (JCV), picornavirus (e.g., Hepatitis A virus), hepadnavirus (e.g., Hepatitis B virus), hepacivirus (e.g., Hepatitis C virus), deltavirus (e.g., Hepatitis D virus), hepevirus (e.g., Hepatitis E virus), or any combination thereof. In preferred embodiments, a sample comprising CTLs (i.e., a PBMC sample) is incubated in culture with the antigenic peptide (e.g., antigenic HPV16 peptides such as those disclosed in Tables 1, 11 and 13) or with antigen-presenting cells (APC) provided herein (e.g., “peptide-pulsed” cells that present said peptide, comprising an HPV epitope described herein, on a class I MHC complex). In some embodiments, the APCs are autologous to the subject from whom the T cells were obtained. In some embodiments, the sample containing T cells is incubated 2 or more times with APCs provided herein. In some embodiments, the T cells are incubated with the APCs in the presence of at least one cytokine. In some embodiments, the cytokine is IL-4, IL-7 and/or IL-15. Exemplary methods for inducing proliferation of T cells using APCs are provided, for example, in U.S. Pat. Pub. No. 2015/0017723, which is hereby incorporated by reference. In some embodiments, the antigens are HPV antigens other than E6 and E7.

In some aspects, provided herein are compositions (e.g., prophylactic and/or therapeutic compositions) comprising the TCR-T cells provided herein. In some embodiments, such compositions are used to treat and/or prevent a cancer, and/or precancerous lesions and/or an HPV infection in a subject by administering to the subject an effective amount of the composition. In some embodiments, the engineered TCR-T cells are not autologous to the subject. In some embodiments, the TCR-T cells are autologous to the subject. In some embodiments, the TCR-T cells are stored in a cell bank before they are administered to the subject. Therefore, in some embodiments, the disclosed immune effector cells that comprise one or more of the engineered TCR polypeptides of the present invention are allogeneic or autologous immune effector cells. Preferably, the allelic HLA restriction (i.e., restriction to a specific HLA-A, HLA-B, or HLA-C allele) of such TCR-T cells is known. In some embodiments, the T cells used for generating the TCR-T cells of the invention are peptide-specific (i.e., sensitized to an antigenic peptide such as a viral peptide). In some embodiments, the T cells used for generating the TCR-T cells of the invention are polyfunctional T cells, i.e., those T cells that are capable of inducing multiple immune effector functions, that provide a more effective immune response to a pathogen than do cells that produce, for example, only a single immune effector (e.g. a single biomarker such as a cytokine or CD107a). Less-polyfunctional, monofunctional, or even “exhausted” T cells may dominate immune responses during chronic infections, thus negatively impacting protection against virus-associated complications. In further preferred embodiments, the TCR-T cells of the invention are polyfunctional. In certain embodiments, at least 50% of the T cells used for generating the TCR-T cells of the invention are CD4⁺ T cells. In some such embodiments, said T cells are less than 50% CD4⁺ T cells. In still further embodiments, said T cells are predominantly CD4⁺ T cells. In some embodiments, at least 50% of the T cells used for generating the TCR-T cells of the invention are CD8⁺ T cells. In some such embodiments, said T cells are less than 50% CD8⁺ T cells. In still further embodiments, said T cells are predominantly CD8⁺ T cells. In some embodiments, the T cells (e.g., the donor samples, the sensitized T cells, and/or TCR-T cells described herein) are stored in a cell library or bank before they are administered to the subject.

In some embodiments, the engineered TCR-T cells expressing the disclosed TCRs further express a dominant-negative mutation that effects immune checkpoint blockade (e.g., express a dominant-negative form of an immune checkpoint molecule such as PD-1). Without intending to be an exhaustive list, the immune checkpoint molecule is selected from programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160. The immune checkpoint molecule may also be transforming growth factor β (TGF-β) receptor. In certain preferred embodiments, the immune checkpoint molecule is CTLA-4. In particularly preferred embodiments, the immune checkpoint molecule is PD-1.

Therapeutic Methods

Immune effector cells expressing the disclosed TCRs can elicit a therapeutically beneficial immune response against HPV antigen-expressing cancer cells (e.g., HPV-associated cancers). For example, an anti-tumor immune response elicited by the disclosed TCR-modified immune effector cells may be an active or a passive immune response. In addition, the TCR-mediated immune response may be part of an adoptive immunotherapy approach in which TCR-modified immune effector cells induce an immune response specific to an HPV antigen, preferably an HPV antigen other than, or in addition to, E6 and E7 antigens.

Adoptive transfer of immune effector cells expressing engineered TCRs is a promising anti-cancer therapy. Accordingly, in some aspects of the invention, provided herein are methods of treating a HPV-associated cancer or precancerous lesions in a subject, the method comprising administering an effective amount of an adoptive immunotherapy composition comprising the TCR-expressing cells contemplated herein. Following the collection of a patient's immune effector cells, the cells may be genetically engineered to express the disclosed HPV antigen-specific TCRs, thus tailoring the specific antigenicity of said immune effector cells (e.g., T cells) and infusing them back into the patient. Moreover, immune effector cells obtained from a donor other than the patient (i.e., allogeneic to the patient) may be genetically engineered to express the disclosed HPV antigen-specific TCRs, then the TCR-containing cells are infused into the patient. In certain specific embodiments, the immune effector cells which comprise an anti-HPV antigen TCR polypeptide are allogeneic HPV-specific cytotoxic T cells.

The disclosed TCR-modified immune effector cells may be administered either alone, or as a pharmaceutical composition in combination with diluents and/or with other components such as IL-2, IL-15, or other cytokines or cell populations. Briefly, pharmaceutical compositions may comprise a targeting cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat MM. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject).

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient by direct administration to an organ, subcutaneously, intradermally, intratumorally, intrathecally, intranodally, intramedullary, intramuscularly, intrapleurally, intracranially, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, provided herein are methods of treating an HPV infection, and/or a cancer, and/or precancerous lesions in a subject comprising administering to the subject a pharmaceutical composition provided herein.

In some embodiments, provided herein is a method of treating an HPV infection in a subject. In certain such embodiments, the subject treated is immunocompromised. For example, the subject may have a T cell deficiency. The subject may have leukemia, lymphoma or multiple myeloma. In some embodiments, the subject is infected with HIV and/or has AIDS. In further embodiments, the subject has undergone a tissue, organ and/or bone marrow transplant. In some such embodiments, the subject is being administered immunosuppressive drugs. In some embodiments, the subject has undergone and/or is undergoing chemotherapy. In some embodiments, the subject has undergone and/or is undergoing radiation therapy.

In certain embodiments, the disclosed TCR-modified immune effector cells are administered to a patient in conjunction with (e.g., before, simultaneously or following) any number of relevant treatment modalities, including but not limited to thalidomide, dexamethasone, bortezomib, and lenalidomide. In further embodiments, the TCR-modified immune effector cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the TCR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In other embodiments, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. The expanded cells may be administered before or after surgery. In some embodiments, the subject is also administered an anti-viral drug that inhibits HPV replication. For example, in some embodiments, the subject is administered podofilox, imiquimod, sinecatechins, podophyllin resin, trichloroacetic acid, or bichloracetic acid. In some embodiments, the subject is also treated with an intervention that physically affects the HPV infected lesions and/or HPV-associated tumors. For example, in some embodiments, the lesions are treated with surgical excision, chemical ablation, cryotherapy, or cauterization.

The cancer of the disclosed methods can be any HPV infected cell (e.g., any HPV antigen-expressing cell) undergoing unregulated growth, invasion, or metastasis in a subject. In some embodiments, the subject has cancer or precancerous lesions. The methods described herein may be used to treat any such cancerous or pre-cancerous lesion. In some embodiments, the cancer and/or precancerous lesions express one or more of the HPV epitopes provided herein (e.g., the HPV epitopes listed in Tables 1, 11 and 13). In some embodiments, the precancerous lesions include abnormal cell changes and/or precancerous cell changes. Precancerous lesions that may be treated by methods and compositions provided herein include, but are not limited to, cervical intraepithelial neoplasia (CIN), squamous intraepithelial lesions (SIL), or warts on the cervix. Cancers that express HPV antigens are known in the art and include, squamous cell carcinomas and solid tumors. Cancers that may be treated by methods and compositions provided herein include, but are not limited to, cancer cells from the cervix, anus, vagina, vulva, penis, tongue base, larynx, tonsil, bladder, blood, bone, bone marrow, brain, breast, colon, esophagus, gastrointestine, gum, head, kidney, liver, lung, nasopharynx, neck, ovary, prostate, skin, non-melanoma skin cancer (NMSC), cutaneous squamous cell carcinoma (SCC), stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

The disclosed TCR-modified immune effector cells (e.g., TCR-T cells) can be used in combination with any compound, moiety or group that has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy. Exemplary anti-cancer compounds include, but are not limited to, Alemtuzumab (Campath®), Alitretinoin (Panretin®), Anastrozole (Arimidex®), Bevacizumab (Avastin®), Bexarotene (Targretin®), Bortezomib (Velcade®), Bosutinib (Bosulif)), Brentuximab vedotin (Adcetris®), Cabozantinib (Cometriq™), Carfilzomib (Kyprolis™), Cetuximab (Erbitux®), Crizotinib (Xalkori®), Dasatinib (Sprycel®), Denileukin diftitox (Ontak®), Erlotinib hydrochloride (Tarceva®), Everolimus (Afinitor®), Exemestane (Aromasin®), Fulvestrant (Faslodex®), Gefitinib (Iressa®), Ibritumomab tiuxetan (Zevalin®), Imatinib mesylate (Gleevec®), Ipilimumab (Yervoy™), Lapatinib ditosylate (Tykerb®), Letrozole (Femara®), Nilotinib (Tasigna®), Ofatumumab (Arzerra®), Panitumumab (Vectibix®), Pazopanib hydrochloride (Votrient®), Pertuzumab (Perjeta™), Pralatrexate (Folotyn®), Regorafenib (Stivarga®), Rituximab (Rituxan®), Romidepsin (Istodax®), Sorafenib tosylate (Nexavar®), Sunitinib malate (Sutent®), Tamoxifen, Temsirolimus (Torisel®), Toremifene (Fareston®), Tositumomab and 131I-tositumomab (Bexxar®), Trastuzumab (Herceptin®), Tretinoin (Vesanoid®), Vandetanib (Caprelsa®), Vemurafenib (Zelboraf®), Vorinostat (Zolinza®), and Ziv-aflibercept (Zaltrap®). Examples of further chemotherapeutic agents include Examples of such chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, especially calicheamicin gammalI and calicheamicin omegal1; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above.

In some embodiments, the subject is also administered an immunotherapeutic agent. Immunotherapy refers to a treatment that uses a subject's immune system to treat cancer, e.g., cancer vaccines, cytokines, use of cancer-specific antibodies, T cell therapy, and dendritic cell therapy.

In some embodiments, the subject is also administered an immune modulatory protein. Examples of immune modulatory proteins include, but are not limited to, B lymphocyte chemoattractant (“BLC”), C-C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), 1-309, Intercellular Adhesion Molecule 1 (“ICAM-1”), Interferon gamma (“IFN-gamma”), Interlukin-1 alpha (“IL-1 alpha”), Interleukin-1 beta (“IL-1 beta”), Interleukin 1 receptor antagonist (“IL-1 ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin-10 (“IL-10”), Interleukin-11 (“IL-11”), Subunit beta of Interleukin-12 (“IL-12 p40” or “IL-12 p70”), Interleukin-13 (“IL-13”), Interleukin-15 (“IL-15”), Interleukin-16 (“IL-16”), Interleukin-17 (“IL-17”), Chemokine (C-C motif) Ligand 2 (“MCP-1”), Macrophage colony-stimulating factor (“M-CSF”), Monokine induced by gamma interferon (“MIG”), Chemokine (C-C motif) ligand 2 (“MIP-1 alpha”), Chemokine (C-C motif) ligand 4 (“MIP-1 beta”), Macrophase inflammatory protein-1-delta (“MIP-1 delta”), Platelet-derived growth factor subunit B (“PDGF-BB”), Chemokine (C-C motif) ligand 5, Regulated on Activation, Normal T cell Expressed and Secreted (“RANTES”), TIMP metallopeptidase inhibitor 1 (“TIMP-1”), TIMP metallopeptidase inhibitor 2 (“TIMP-2”), Tumor necrosis factor, lymphotoxin-alpha (“TNF alpha”), Tumor necrosis factor, lymphotoxin-beta (“TNF beta”), Soluble TNF receptor type 1 (“sTNFRI”), sTNFRIIAR, Brain-derived neurotrophic factor (“BDNF”), Basic fibroblast growth factor (“bFGF”), Bone morphogenetic protein 4 (“BMP-4”), Bone morphogenetic protein 5 (“BMP-5”), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”), Neurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing complex (“SCF”), Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“VEGF”), Vascular endothelial growth factor receptor 2 (“VEGFR2”), Vascular endothelial growth factor receptor 3 (“VEGFR3”), VEGF-D 6Ckine, Tyrosine-protein kinase receptor UFO (“Axl”), Betacellulin (“BTC”), Mucosae-associated epithelial chemokine (“CCL28”), Chemokine (C-C motif) ligand 27 (“CTACK”), Chemokine (C-X-C motif) ligand 16 (“CXCL16”), C-X-C motif chemokine 5 (“ENA-78”), Chemokine (C-C motif) ligand 26 (“Eotaxin-3”), Granulocyte chemotactic protein 2 (“GCP-2”), GRO, Chemokine (C-C motif) ligand 14 (“HCC-1”), Chemokine (C-C motif) ligand 16 (“HCC-4”), Interleukin-9 (“IL-9”), Interleukin-17 F (“IL-17F”), Interleukin-18-binding protein (“IL-18 BPa”), Interleukin-28 A (“IL-28A”), Interleukin 29 (“IL-29”), Interleukin 31 (“IL-31”), C-X-C motif chemokine 10 (“IP-10”), Chemokine receptor CXCR3 (“I-TAC”), Leukemia inhibitory factor (“LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C-C motif) ligand 20 (“MIP-3 alpha”), C-C motif chemokine 19 (“MIP-3 beta”), Chemokine (C-C motif) ligand 23 (“MPIF-1”), Macrophage stimulating protein alpha chain (“MSPalpha”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor-1 alpha (“SDF-1 alpha”), Chemokine (C-C motif) ligand 17 (“TARC”), Thymus-expressed chemokine (“TECK”), Thymic stromal lymphopoietin (“TSLP 4-IBB”), CD 166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”), Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta, IL-17R, IL-2Rgamma, IL-21R, Lysosome membrane protein 2 (“LIMPII”), Neutrophil gelatinase-associated lipocalin (“Lipocalin-2”), CD62L (“L-Selectin”), Lymphatic endothelium (“LYVE-1”), MHC class I polypeptide-related sequence A (“MICA”), MHC class I polypeptide-related sequence B (“MICB”), NRG1-betal, Beta-type platelet-derived growth factor receptor (“PDGF Rbeta”), Platelet endothelial cell adhesion molecule (“PECAM-1”), RAGE, Hepatitis A virus cellular receptor 1 (“TIM-1”), Tumor necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDAR, Activin A, Agouti-related protein (“AgRP”), Ribonuclease 5 (“Angiogenin”), Angiopoietin 1, Angiostatin, Cathepsin S, CD40, Cryptic family protein IB (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C, FoUistatin, Galectin-7, Intercellular adhesion molecule 2 (“ICAM-2”), IL-13 R1, IL-13R2, IL-17B, IL-2 Ra, IL-2 Rb, IL-23, LAP, Neuronal cell adhesion molecule (“NrCAM”), Plasminogen activator inhibitor-1 (“PAI-1”), Platelet derived growth factor receptors (“PDGF-AB”), Resistin, stromal cell-derived factor 1 (“SDF-1 beta”), sgpl30, Secreted frizzled-related protein 2 (“ShhN”), Sialic acid-binding immunoglobulin-type lectins (“Siglec-5”), ST2, Transforming growth factor-beta 2 (“TGF beta 2”), Tie-2, Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFR1, Adiponectin, Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”), Carcinoembryonic antigen (“CEA”), cAMP receptor protein (“CRP”), Human Epidermal Growth Factor Receptor 2 (“ErbB2”), Follistatin, Follicle-stimulating hormone (“FSH”), Chemokine (C-X-C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, Tumor necrosis factor ligand superfamily member 13/Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGF R”), IFN-alpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD-1”), N-acetylmuramoyl-L-alanine amidase (“PGRP-5”), Serpin A4, Secreted frizzled related protein 3 (“sFRP-3”), Thrombomodulin, Toll-like receptor 2 (“TLR2”), Tumor necrosis factor receptor superfamily member 10A (“TRAIL R1”), Transferrin (“TRF”), WIF-1ACE-2, Albumin, AMICA, Angiopoietin 4, B-cell activating factor (“BAFF”), Carbohydrate antigen 19-9 (“CA19-9”), CD 163, Clusterin, CRT AM, Chemokine (C-X-C motif) ligand 14 (“CXCL14”), Cystatin C, Decorin (“DCN”), Dickkopf-related protein 3 (“Dkk-3”), Delta-like protein 1 (“DLL1”), Fetuin A, Heparin-binding growth factor 1 (“aFGF”), Folate receptor alpha (“FOLR1”), Furin, GPCR-associated sorting protein 1 (“GASP-1”), GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST, Heparan sulfate proteoglycan (“Syndecan-1”), Tumor necrosis factor receptor superfamily member 13B (“TACI”), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNT1-inducible-signaling pathway protein 1 (“WISP-1”), and Receptor Activator of Nuclear Factor κ B (“RANK”). In certain preferred embodiments, the subject is also administered IFN-gamma (IFNγ). In particularly preferred embodiments, the subject is pretreated with IFNγ, such as with low doses of IFNγ, prior to administering the TCR-modified immune effector cells disclosed herein (e.g., the adoptive immunotherapy compositions disclosed herein comprising the TCR-T cells disclosed herein).

The disclosed TCRs can be used in combination with an immune checkpoint inhibitor. Immune Checkpoint inhibition broadly refers to inhibiting the checkpoints that cancer cells can produce to prevent or downregulate an immune response. Two known immune checkpoint pathways involve signaling through the cytotoxic T-lymphocyte antigen-4 (CTLA-4) and programmed-death 1 (PD-1) receptors. These proteins are members of the CD28-B7 family of co-signaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T cell proliferation. Checkpoint inhibitors include, but are not limited to aptamers and antibodies that block PD-1 (Nivolumab (BMS-936558 or MDX1106), CT-011, MK-3475, AMP-514), PD-L1 (MDX-1105 (BMS-936559), MPDL3280A, MSB0010718C), PD-L2 (rHIgM12B7, AMP-224), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD-1 inhibitor comprises an antibody that specifically binds PD-1, such as lambrolizumab (Merck), nivolumab (Bristol-Myers Squibb), or MEDI4736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed TCRs can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbB1 (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM1 or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec STI571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with a TCRs for treating the disorders as described above may be an anti-cancer cytokine, chemokine, or combination thereof. Examples of suitable cytokines and growth factors include IFNγ, IL-2, IL-4, IL-6, IL-7, IL-10, IL-12, IL-13, IL-15, IL-18, IL-23, IL-24, IL-27, IL-28a, IL-28b, IL-29, KGF, IFNα, (e.g., INFa2b), IFNβ, GM-CSF, CD40L, Flt3 ligand, stem cell factor, ancestim, and TNFα. Suitable chemokines may include Glu-Leu-Arg (ELR)-negative chemokines such as IP-10, MCP-3, MIG, and SDF-1a from the human CXC and C-C chemokine families. Suitable cytokines include cytokine derivatives, cytokine variants, cytokine fragments, and cytokine fusion proteins.

In some embodiments, a therapeutic agent for use in combination with a CARs for treating the disorders as described above may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent for use in combination with a TCRs for treating the disorders as described above may be a hormonal regulating agent (e.g., hormone therapy), such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogen (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent for use conjointly with TCRs for treating the disorders as described above may be an anti-cancer nucleic acid or an anti-cancer inhibitory RNA molecule.

In some embodiments, the disclosed TCRs is administered conjointly with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed TCRs are administered conjointly with surgery.

### EXEMPLIFICATION

### Example 1: Generation of HPV-Specific T Cell Lines

Cryopreserved, HPV⁺ PBMCs from HNSCC patients were washed and re-suspended in complete RPMI medium and incubated overnight (i.e., at 37° C., 6.5% CO₂). Cells (5×10⁶) were stimulated with HPV16 antigen epitope at a concentration of 1 μg/ml and incubated for an hour. The cells were then washed and further grown (i.e., incubated) for 14 days in 24 well plates. The cultures were supplemented with R10 medium containing interleukin-2 (IL-2) at 200 IU/ml on day 2 and then every 3 days thereafter, until 14 days. On day 14, viable T cells were counted by trypan blue exclusion and subsequently cryopreserved in liquid nitrogen.

### Example 2: Sorting of Antigen-Specific CTLs by Dual Cytokine Capture Assay

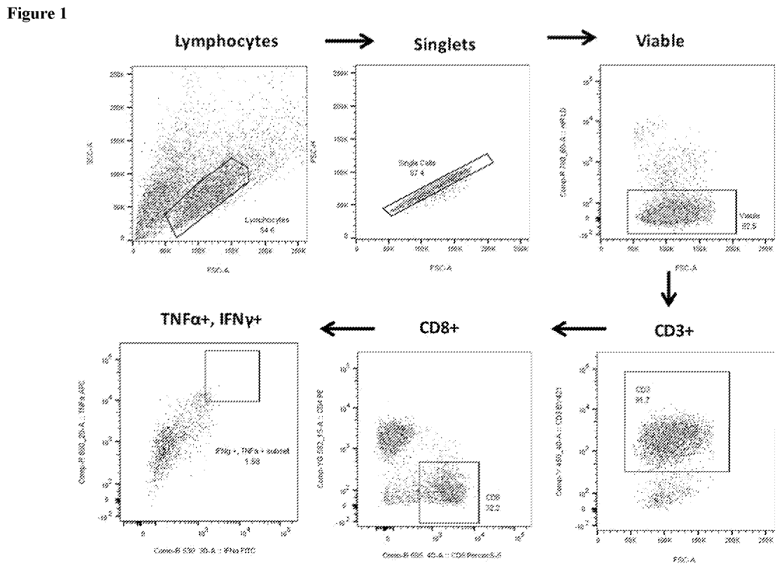

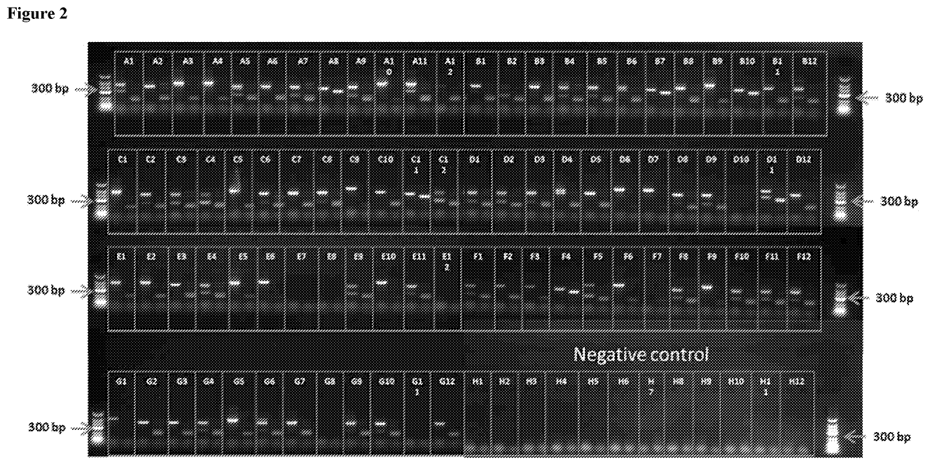

The cryopreserved T cells (10×10⁶) were rapidly thawed, washed and re-suspended in 10 ml complete medium containing 120 IU/ml of IL-2 and incubated overnight. A 1 ml aliquot of cells from the overnight culture was used as a “stimulator” (e.g., antigen-presenting) population. To the stimulator culture, 1 μg/ml of the antigenic peptide (e.g., the corresponding peptide selected from SEQ ID NOs. 1-12, 217, and 218) was added and incubated for 1 hour; followed by three washes with complete medium. The stimulator cells were then added to CTLs (i.e., “responder” cells) at a 1:9 ratio, into 48 wells (approx. 5-10×10⁶ per well) and incubated for 3 hours. Following incubation, CTLs were washed in ice cold buffer (PBS, 2 mM EDTA, and 0.5% BSA) and centrifuged at 400 g for 10 minutes at 4° C. Cells were then re-suspended in 80 μl of cold complete medium and 10 μl of IFNγ and TNFα catch reagent (anti-cytokine antibody conjugated to a cell surface-specific antibody, Miltenyi Biotec; Bergisch Gladbach, Germany) per 10⁶ cells and incubate on ice for 5 minutes. Cells were then centrifuged in warm complete medium to dilute cells to 10⁵ cells/ml and tubes were gently mixed (e.g., manually turning tubes every five minutes or placing in rotating mixer such as a the Miltenyi MACSmix™, at low rotation setting) in a 37° C. incubator for 45 mins. After incubation, each tube was topped-up with ice cold buffer and centrifuged (300 g, 10 minutes, 4° C.) followed by one more wash with cold buffer. The cells were re-suspended in 80 μl of cold buffer per 10⁶, containing ×2.5 μl of anti-CD3 BV421, ×1 μl of anti-CD8 perCPCy5.5, ×2.5 μl of anti-CD4 PE, ×0.2 μl NIR live-dead, ×10 μl IFNγ detection antibody PE, ×10 μl of TNFα detection antibody APC and incubated on ice for 10 minutes followed by wash in ice cold buffer. Cells were then re-suspended in 300 μl cold buffer and filtered (0.45 μm) and used for fluorescence-activated cell sorting (FACS). Single, IFNγ and TNFα double-positive HPV-specific CD8⁺ T cells were sorted into individual wells of a 96-well PCR plate (FIG. 1), the last row of wells remaining empty for use as PCR negative controls. After sorting, plates were stored at −80° C. until downstream processing.

### Example 3: RT-PCR, Multiplex Nested PCR, and Sequencing

Complementary DNA (cDNA) was synthesized from single cells in the 96-well PCR plates using the SuperScript™ VILO™ cDNA Synthesis Kit (Invitrogen™, Thermo Fisher Scientific; MA, U.S.A.) in 2.5 μl reaction mixes, each containing 0.5 μl of 5×VILO reaction mix, 0.25 μl of 10× Superscript reverse transcriptase, and 0.1% Triton X-100.

TCR transcripts from each cell were amplified by multiplex nested PCR in 25 μl reaction mixes containing 2.5 μl of cDNA. Primers of 17- to 23-base pair length were designed to target each family of related V genes and the TRAC and TRBC genes, allowing for up to three degenerate base pairs. A total of 40 external/internal pairs of sense TRAV, 27 sense TRBV, and 1 each of antisense TRAC and TRBC gene segment-specific primers were generated (Table 12). For inclusion in nested PCRs, TRAV and TRBV primers were multiplexed to a concentration of 5 μM for each primer, and TRAC and TRBC primers were reconstituted to a concentration of 5 μM. The first-round (external) PCR was performed with 0.75 U of Taq DNA polymerase, 2.5 μl of 10×PCR Buffer (containing KCl, (NH₄)₂SO₄, and 15 mM MgCl₂, 0.5 μl of 10 mM deoxynucleotide triphosphates (dNTPs), 0.1 μM each of the external sense TRAV (T cell receptor α variable) and TRBV (T cell receptor β variable) primers, and each of the external antisense TRAC (T cell receptor α constant) and TRBC (T cell receptor β constant) primers listed in Table 12. Aliquots (2.5 μl) of the external PCR products served as templates for two separate second-round (internal) PCRs in 25 μl reactions that incorporated, respectively, either
(i) internal sense TRAV primers and internal antisense TRAC primer or
(ii) internal sense TRBV primers and internal antisense TRBC primer, listed in Table 12.
   Instead of 10×PCR Buffer, the second-round PCR used CoralLoad® PCR Buffer (Qiagen GmbH; Hilden, Germany) containing two marker dyes for gel loading. Internal PCR product (5 μl) was run on 2% agarose gel at 80V to select the positive wells for further analysis (FIG. 2).

TABLE 12
PCR primers

| Gene | External PCR | SEQ ID | Internal PCR | SEQ ID |
|---|---|---|---|---|
| name | primer Sequence | NO. | primer Sequence | NO. |
| TRAV1 | AACTGCACGTACCAGACATC | 71 | GCACCCACATTTCTKTCTTAC | 140 |
| TRAV2 | GATGTGCACCAAGACTCC | 72 | CACTCTGTGTCCAATGCTTAC | 141 |
| TRAV3 | AAGATCAGGTCAACGTTGC | 73 | ATGCACCTATTCAGTCTCTGG | 142 |
| TRAV4 | CTCCATGGACTCATATGAAGG | 74 | ATTATATCACGTGGTACCAACAG | 143 |
| TRAV5 | CTTTTCCTGAGTGTCCGAG | 75 | TACACAGACAGCTCCTCCAC | 144 |
| TRAV6 | CACCCTGACCTGCAACTATAC | 76 | TGGTACCGACAAGATCCAG | 145 |
| TRAV7 | AGCTGCACGTACTCTGTCAG | 77 | ACAATTTGCAGTGGTACAGG | 146 |
| TRAV8-1 | CTCACTGGAGTTGGGATG | 78 | GTCAACACCTTCAGCTTCTC | 147 |
| TRAV8-2 | GCCACCCTGGTTAAAGG | 79 | AGAGTGAAACCTCCTTCCAC | 148 |
| TRAV8-3 | CACTGTCTCTGAAGGAGCC | 80 | TTTGAGGCTGAATTTAAGAGG | 149 |
| TRAV8-6 | GAGCTGAGGTGCAACTACTC | 81 | AACCAAGGACTCCAGCTTC | 150 |
| TRAV8-7 | CTAACAGAGGCCACCCAG | 82 | ATCAGAGGTTTTGAGGCTG | 151 |
| TRAV9-1 | TGGTATGTCCAATATCCTGG | 83 | GAAACCACTTCTTTCCACTTG | 152 |
| TRAV10 | CAAGTGGAGCAGAGTCCTC | 84 | GAAAGAACTGCACTCTTCAATG | 153 |
| TRAV12-1 | CARTGTTCCAGAGGGAGC | 85 | AAGATGGAAGGTTTACAGCAC | 154 |
| TRAV13-1 | CATCCTTCAACCCTGAGTG | 86 | TCAGACAGTGCCTCAAACTAC | 155 |
| TRAV13-2 | CAGCGCCTCAGACTACTTC | 87 | CAGTGAAACATCTCTCTCTGC | 156 |
| TRAV14 | AAGATAACTCAAACCCAACCAG | 88 | AGGCTGTGACTCTGGACTG | 157 |
| TRAV16 | AGTGGAGCTGAAGTGCAAC | 89 | GTCCAGTACTCCAGACAACG | 158 |
| TRAV17 | GGAGAAGAGGATCCTCAGG | 90 | CCACCATGAACTGCAGTTAC | 159 |
| TRAV18 | TCCAGTATCTAAACAAAGAGCC | 91 | TGACAGTTCCTTCCACCTG | 160 |
| TRAV19 | AGGTAACTCAAGCGCAGAC | 92 | TGTGACCTTGGACTGTGTG | 161 |
| TRAV20 | CACAGTCAGCGGTTTAAGAG | 93 | TCTGGTATAGGCAAGATCCTG | 162 |
| TRAV21 | TTCCTGCAGCTCTGAGTG | 94 | AACTTGGTTCTCAACTGCAG | 163 |
| TRAV22 | GTCCTCCAGACCTGATTCTC | 95 | CTGACTCTGTGAACAATTTGC | 164 |
| TRAV23 | TGCTTATGAGAACACTGCG | 96 | TGCATTATTGATAGCCATACG | 165 |
| TRAV24 | CTCAGTCACTGCATGTTCAG | 97 | TGCCTTACACTGGTACAGATG | 166 |
| TRAV25 | GGACTTCACCACGTACTGC | 98 | TATAAGCAAAGGCCTGGTG | 167 |
| TRAV26-1 | GCAAACCTGCCTTGTAATC | 99 | CGACAGATTCACTCCCAG | 168 |
| TRAV26-2 | AGCCAAATTCAATGGAGAG | 100 | TTCACTTGCCTTGTAACCAC | 169 |
| TRAV27 | TCAGTTTCTAAGCATCCAAGAG | 101 | CTCACTGTGTACTGCAACTCC | 170 |
| TRAV29 | GCAAGTTAAGCAAAATTCACC | 102 | CTGCTGAAGGTCCTACATTC | 171 |
| TRAV30 | CAACAACCAGTGCAGAGTC | 103 | AGAAGCATGGTGAAGCAC | 172 |
| TRAV34 | AGAACTGGAGCAGAGTCCTC | 104 | ATCTCACCATAAACTGCACG | 173 |
| TRAV35 | GGTCAACAGCTGAATCAGAG | 105 | ACCTGGCTATGGTACAAGC | 174 |
| TRAV36 | GAAGACAAGGTGGTACAAAGC | 106 | ATCTCTGGTTGTCCACGAG | 175 |
| TRAV38-1 | GCACATATGACACCAGTGAG | 107 | CAGCAGGCAGATGATTCTC | 176 |
| TRAV39 | CTGTTCCTGAGCATGCAG | 108 | TCAACCACTTCAGACAGACTG | 177 |
| TRAV40 | GCATCTGTGACTATGAACTGC | 109 | GGAGGCGGAAATATTAAAGAC | 178 |
| TRAV41 | AATGAAGTGGAGCAGAGTCC | 110 | TTGTTTATGCTGAGCTCAGG | 179 |
| TRAC | GACCAGCTTGACATCACAG | 111 | TGTTGCTCTTGAAGTCCATAG | 180 |
| TRBV2 | TCGATGATCAATTCTCAGTTG | 112 | TTCACTCTGAAGATCCGGTC | 181 |
| TRBV3-1 | CAAAATACCTGGTCACACAG | 113 | AATCTTCACATCAATTCCCTG | 182 |
| TRBV4-1 | TCGCTTCTCACCTGAATG | 114 | CCTGCAGCCAGAAGACTC | 183 |
| TRBV5-1 | GATTCTCAGGKCKCCAGTTC | 115 | CTTGGAGCTGGRSGACTC | 184 |
| TRBV5-5 | GTACCAACAGGYCCTGGGT | 116 | TCTGAGCTGAATGTGAACG | 185 |
| TRBV6-1 | ACTCAGACCCCAAAATTCC | 117 | GTGTRCCCAGGATATGAACC | 186 |
| TRBV6-4 | ACTGGCAAAGGAGAAGTCC | 118 | TGGTTATAGTGTCTCCAGAGC | 187 |
| TRBV7-1 | TRTGATCCAATTTCAGGTCA | 119 | TCYACTCTGAMGWTCCAGCG | 188 |
| TRBV7-4 | GSWTCTYTGCAGARAGGCC | 120 | TGRMGATYCAGCGCACA | 189 |
| TRBV9 | GATCACAGCAACTGGACAG | 121 | GTACCAACAGAGCCTGGAC | 190 |
| TRBV10-1 | TGTWCTGGTATCGACAAGACC | 122 | TCCYCCTCACTCTGGAGTC | 191 |
| TRBV11-1 | CGATTTTCTGCAGAGACGC | 123 | GACTCCACTCTCAAGATCCA | 192 |
| TRBV12-3 | ARGTGACAGARATGGGACAA | 124 | CYACTCTGARGATCCAGCC | 193 |
| TRBV13 | AGCGATAAAGGAAGCATCC | 125 | CATTCTGAACTGAACATGAGC | 194 |
| TRBV14 | CCAACAATCGATTCTTAGCTG | 126 | ATTCTACTCTGAAGGTGCAGC | 195 |
| TRBV15 | AGTGACCCTGAGTTGTTCTC | 127 | ATAACTTCCAATCCAGGAGG | 196 |
| TRBV16 | GTCTTTGATGAAACAGGTATGC | 128 | GAAAGATTTTCAGCTAAGTGCC | 197 |
| TRBV17 | CAGACCCCCAGACACAAG | 129 | TGTTCACTGGTACCGACAG | 198 |
| TRBV18 | CATAGATGAGTCAGGAATGCC | 130 | CGATTTTCTGCTGAATTTCC | 199 |
| TRBV19 | AGTTGTGAACAGAATTTGAACC | 131 | TTCCTCTCACTGTGACATCG | 200 |
| TRBV20-1 | AAGTTTCTCATCAACCATGC | 132 | ACTCTGACAGTGACCAGTGC | 201 |
| TRBV23-1 | GCGATTCTCATCTCAATGC | 133 | GCAATCCTGTCCTCAGAAC | 202 |
| TRBV24-1 | CCTACGGTTGATCTATTACTCC | 134 | GATGGATACAGTGTCTCTCGA | 203 |
| TRBV25-1 | ACTACACCTCATCCACTATTCC | 135 | CAGAGAAGGGAGATCTTTCC | 204 |
| TRBV27 | TGGTATCGACAAGACCCAG | 136 | TTCYCCCTGATYCTGGAGTC | 205 |
| TRBV29-1 | TTCTGGTACCGTCAGCAAC | 137 | TCTGACTGTGAGCAACATGAG | 206 |
| TRBV30 | TCCAGCTGCTCTTCTACTCC | 138 | AGAATCTCTCAGCCTCCAGAC | 207 |
| TRBC | TAGAACTGGACTTGACAGCG | 139 | TTCTGATGGCTCAAACACAG | 208 |

These positive PCR products were purified and used as templates for dye-terminator sequencing. Each reaction mixture, comprising 0.5 μM of TRAC or TRBC internal primer, was added to a 96-well plate containing ˜7 μl purified PCR product (˜20 μl total/well), and the reactions underwent PCR thermocycling. Extension sequencing product was purified by ethanol precipitation and samples were used for capillary electrophoresis sequencing. Sequence from TCRα and TCRβ plates were analyzed by IMGT/V-QUEST, an integrated alignment tool for immunoglobulin and T cell receptor nucleotide sequences, available online from the international ImMunoGeneTics Information System® (IMGT®) home page. Briefly, nucleotide sequence from each sequenced well was entered into the IMGT/V-QUEST online tool as a T cell receptor (TR) nucleotide sequence and IMGT/V-QUEST identified the CDR3 region, the V, D, and J genes, and alleles by alignment of the input sequence with the IMGT reference directory. Out of sequenced clones, the maximum co-expressed TCRα and TCRβ sequences were selected and used to generate TCR lentivirus constructs.

Results

The identified and sequenced clones (i.e., HPV-specific TCR amino acid sequences), whose expression and functional activity was checked in Jurkat cells (primary screening) and in PBMCs (secondary screening) are represented below in Table 13.

TABLE 13
HPV-specific TCRs sequences

| HPV16 | | HLA | TCRα | TCRβ | CDR3 sequence |
|---|---|---|---|---|---|
| Antigen | Epitope | restriction | gene | gene | (α/β) |
| E2 | TLQDVSLEV | A*02:01 | TRAV38- | | CDR3α sequence: |
| | YL (SEQ | | 01*03 | | CAFTYGGSQGNLIF |
| | NO. 3) | | TRAJ42-01 | | (SEQ ID NO. 25) |
| | | | | TRBV2*01 | CDR3β sequence: |
| | | | | TRBJ2-2*01 | CASRASVGVGTGELFF |
| | | | | TRBD1*01 | (SEQ ID NO. 26) |
| E5 | NLDTASTTL | C*05:01 & | TRAV19-01 | | CDR3α sequence: |
| | (SEQ ID | C*08:02 | TRAJ42-01 | | CALSEGGGSQGNLIF |
| | NO. 1) | | | | (SEQ ID NO. 13) |
| | | | | TRBV7-6*01 | CDR3β sequence: |
| | | | | TRBJ2-5*01 | CASSPELAGPQETQYF |
| | | | | TRBD2*01 | (SEQ ID NO. 14) |
| E6 | AFRDLCIVY | C*07:02 | TRAV19*01 | | CDR3α sequence: |
| | (SEQ ID | | TRAJ40*01 | | CALGSSGTYKYI |
| | NO. 5) | | | | (SEQ ID NO. 39) |
| | | | | TRBV25-1*01 | CDR3β sequence: |
| | | | | TRBJ1-5*01 | CASSGPGQGHNQPQHF |
| | | | | TRBD1*01 | (SEQ ID NO. 40) |
| E6 | TIHDIILEC | A*02:01 | TRAV1-2*01 | | CDR3α sequence: |
| | V (SEQ ID | | TRAJ26*01 | | CAVRDTGYGQNFVF |
| | NO. 4) | | | | (SEQ ID NO. 31) |
| | | | | TRBV27*01 | CDR3β sequence: |
| | | | | TRBJ1-6*02 | CASSPQGRINSPLHF |
| | | | | TRBD1*01 | (SEQ ID NO. 32) |
| E6 | HDIILECVY | B*18:01 | TRAV26-1*01 | | CDR3α sequence: |
| | (SEQ ID | (B44 super | TRAJ26*01 | | CIVRDRSYGQNFVF |
| | NO. 2) | family) | | | (SEQ ID NO. 19) |
| | | | | TRBV20-1*01 | CDR3β sequence: |
| | | | | TRBJ2-1*01 | CSAREGYRSYF |
| | | | | TRBD1*01 | (SEQ ID NO. 20) |
| E6 | KQRFHNIRG | DQB1*03:01/ | TRAV13-2*01, | | CDR3α sequence: |
| | RWTGRC | DRB1*15:01 | TRAJ23*01 | | CAETLGLDQGGKLIF |
| | (SEQ ID | | | | (SEQ ID NO. 43) |
| | NO. 6) | | | TRBV20-1*03 | CDR3β sequence: |
| | | | | TRBJ2-3*01 | CSTAGETDTQYF |
| | | | | TRBD2*01 | (SEQ ID NO. 44) |

### Example 4: TCR Lentivirus Constructs

TCR nucleotide sequences were synthesized and a vector insert was cloned into the pLV lentivirus backbone. The insert sequence was codon optimized for expression in human tissues. For partial murinization of human constant region, some amino acids were replaced with mouse constant regions. For the T cell receptor α constant region amino acid Pro91, Glu92, Ser93, Ser94 (see International ImMunoGeneTics Information System®, Accession #X02883|TRAC*01|Homo sapiens) were replaced with Ser, Asp, Val, Pro amino acid respectively. For T cell receptor β constant region Glu18, Ser22, Phe133, Glu136, Gln139 (see International ImMunoGeneTics Information System®, Accession #'s M12887|TRBC1*01|Homo sapiens and M12888|TRBC2*01|Homo sapiens) were replaced with Lys, Ala, Ile, Ala, His amino acid respectively. For constructs with an additional interchain disulfide bond, cysteine was substituted in place of Thr48 of the α chain and Ser57 of the β chain. The vector inserts were also designed to encode the α- and β-chains of the identified TCR (with the constant regions of each TCR chain partially murinized) linked by a furin-2A self-cleaving peptide.

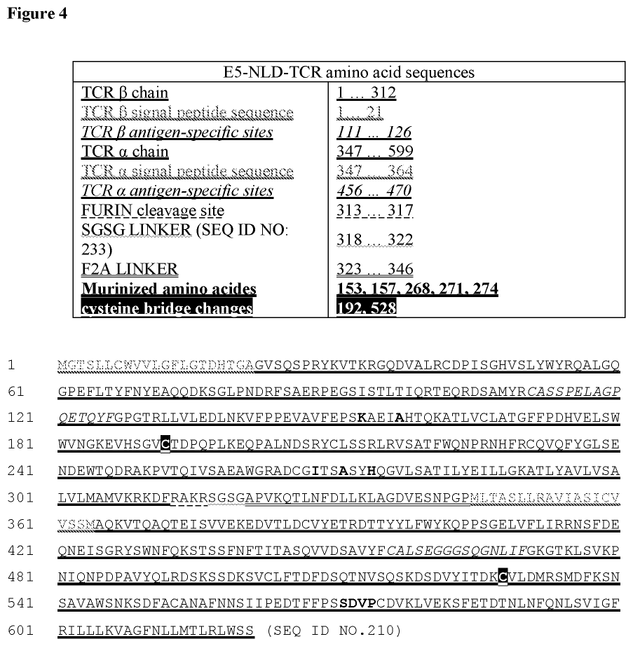

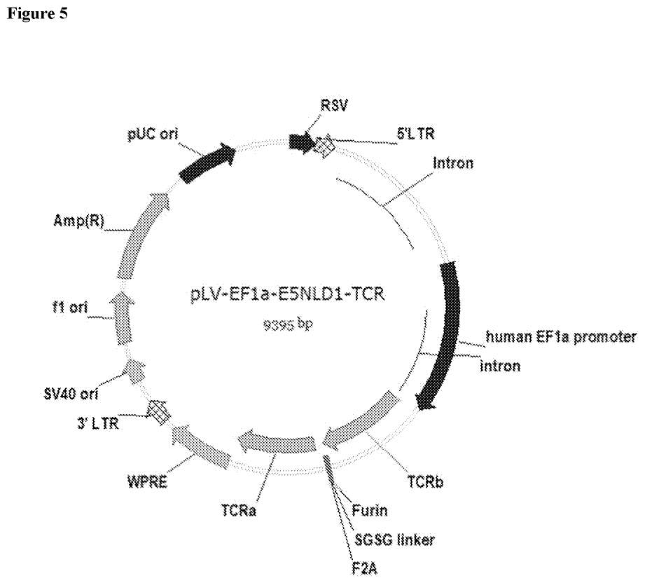

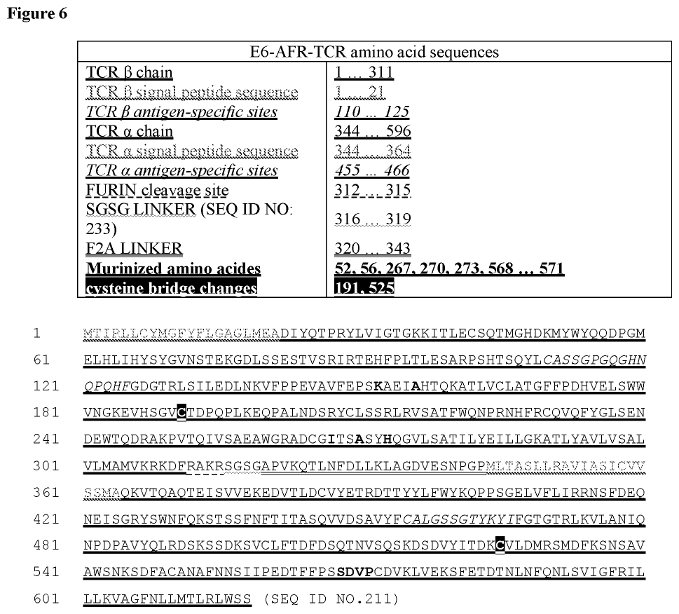

The nucleotide sequence for each TCR lentivirus construct, and its relevant features, are illustrated in Tables 2 to 11. Similarly, the amino acid sequence of each expressed TCR construct, and it's sequence features, is described, respectively, in FIG. 3, FIG. 4, and FIGS. 6-11.

### Example 5: Jurkat Cells Engineered to Express 1a HPV-TCR from Different HPV Antigens (E2, E5, and E6) and Displaying Antigen Recognition (Primary Screening)

TCR lentivirus supernatants were generated by co-transfection of 293T cells with TCR pLV vector and packaging plasmid (pVSV, pMDL and pREV). Two days after transfection, TCR lentiviral supernatants were harvested.

40,000 Jurkat cells in 40 μl were transduced with lentivirus (Multiplicity of infection (MOI) of 50) in 96 well plates. Transduction was checked at day 3, post-transduction, by measuring TCR expression by flow cytometry.

Transduced Jurkat cells were further confirmed to have antigen specificity and HLA restriction of the engineered TCR. Briefly, lymphoblastoid cells (LCLs) were stimulated with peptide (1 μg/ml) in R-0 (no FBS) media and incubated for 1 hour. The stimulated, antigen-presenting LCLs were then washed (5×) with complete RPMI medium 2300 rpm for 2 min. The transduced Jurkat cells (2×10⁵/well) were then co-cultured with an equal number of the peptide-pulsed LCLs (either HLA-matched or mismatched) in complete medium in 96-well U-bottom plates and incubated for 24 hours and analyzed by flow cytometry.

For cytometry analysis cells were washed with PBS containing 2% FBS (wash buffer) and the pellet was re-suspended in 50 μL of wash buffer containing FITC-conjugated anti-Vα12.1 (E5-NLD TCR, clone-6D6.6; Thermofisher) or PE-conjugated anti-Vβ2 (E6-HDI, clone-MPB2D5, Beckman Coulter), anti-Vβ11 (E6-AFR-TCR, C21; Beckman Coulter), anti-Vβ14 (E6-TIH-TCR, clone-REA557; Miltenyi Biotec), or anti-Vβ22 (E2-TLQ-TCR, clone-IMMU 546; Beckman Coulter); and Near IR live-dead exclusion dye, and then incubated at 4° C. for 30 minutes. Cells were then washed twice with wash buffer, re-suspended in PBS containing 1% paraformaldehyde prior to analysis.

Results

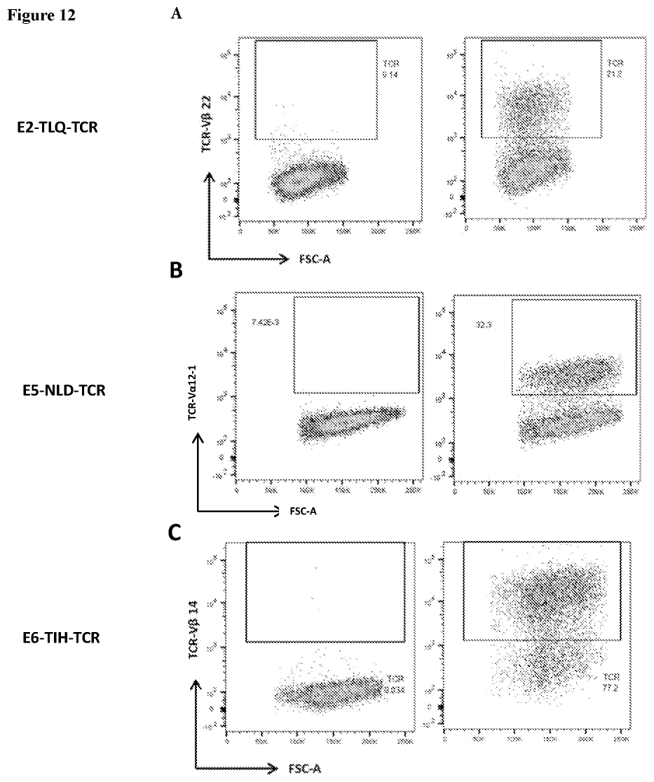

By day 3, post transduction with E2-TLQ-TCR, E5-NLD-TCR, E6-TIH-TCR lentivirus, Jurkat cells showed TCR expression as detected by anti-Vβ22, Vα12.1, and Vβ14 antibodies, respectively (see FIG. 12).

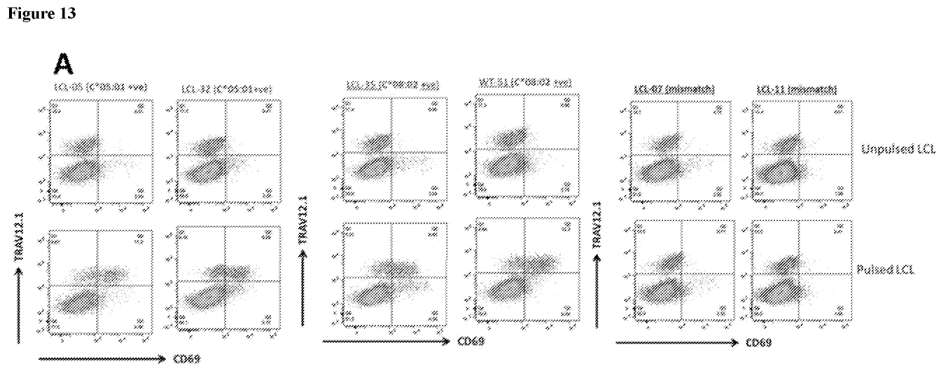

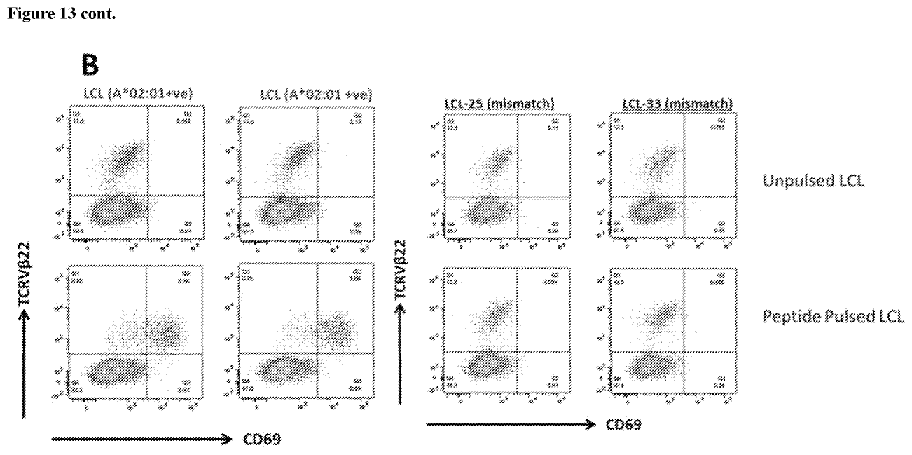

Similarly, Jurkat cells transduced with E5-NLD-TCR (restricted to HLA-C*05:01 and C*08:02) and E2-TLQ-TCR (restricted to HLA-A*02:01) exhibit CD69 expression (as detected with anti-human CD69 antibody, Clone-FN50) after co-incubation with peptide pulsed LCL (see FIG. 13).

### Example 6: T Cells Engineered to Express a TCR from E5 Antigen (E5-NLD-TCR) Displayed Multifunctional Activity and High Functional Avidity (Secondary Screening)

Human peripheral blood mononuclear cells (PBMCs) were isolated from healthy donors. Prior to transduction, PBMCs were cultured in complete media with CD3 and CD28 agonists to provide primary and co-stimulatory signals for optimized and efficient T cell activation and expansion for 2 days.

Transductions were performed by adding lentiviral supernatant (up to 80-90 μl well) to RetroNectin®-coated 96-well plates (Takara Bio USA, Inc.; CA, U.S.A.). The plates were centrifuged for 2 hours at 2,000 g and 32° C. The supernatant was discarded and the plates were washed with PBS. PBMCs were added (20,000/well) and the plates were centrifuged at 1,500 rpm for 10 minutes at 32° C. After approximately 16 hours, the cells were transferred to 24-well tissue culture-treated plates and cultured for 7 days in the presence of IL-2 (200 IU/ml). At day 8, transduced T cells were re-stimulated with peptide-pulsed autologous PBMCs at a 2:1 effector-to-target ratio and cultured in complete RPMI media. IL-2 was added at 2001 U/ml on day 2 and then every 3 days thereafter until 21 days. On days 7 and 14, post re-stimulation, viable T cells were visually counted by Trypan Blue exclusion and used for TCR expression studies and functional assays. Any remaining cells were cryopreserved.

The transduced T cells were incubated with cognate peptide antigen in R10 media containing monensin, brefeldin and anti-CD107a antibody. After 4 hrs incubation, the cells were washed with PBS containing 2% FBS (wash buffer) and the pellet was re-suspended in wash buffer containing FITC-conjugated anti-CD4 and PerCP-Cy5.5 conjugated anti-CD8 antibodies for IFNγ analysis, or with PerCP-Cy5.5-conjugated anti-CD8 and PE-Cy7-conjugated anti-CD4 and then incubated at 4° C. for 30 minutes. Cells were then washed twice with PBS, fixed and permeabilized for 20 min. Cells were then washed and incubated with PE-conjugated anti-IL-2, Alexa Fluor 700-conjugated anti-IFNγ and APC-conjugated anti-TNF 4° C. for 30 minutes. Stained cells were washed twice with permeabilizing wash buffer, resuspended in PBS containing 1% paraformaldehyde and analyzed by flow cytometry.

Results

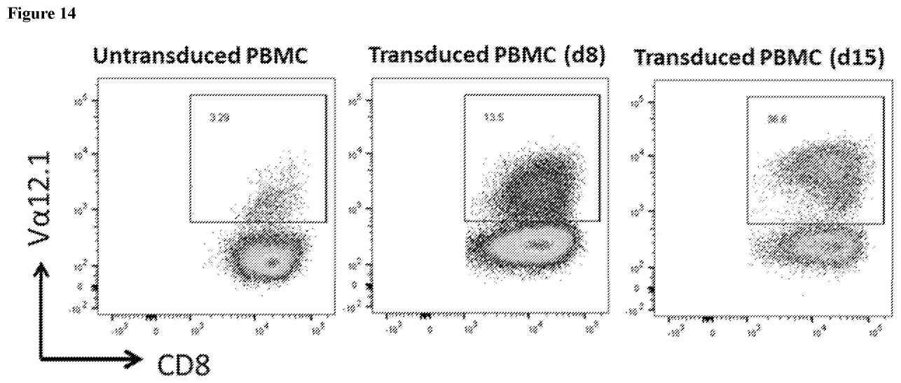

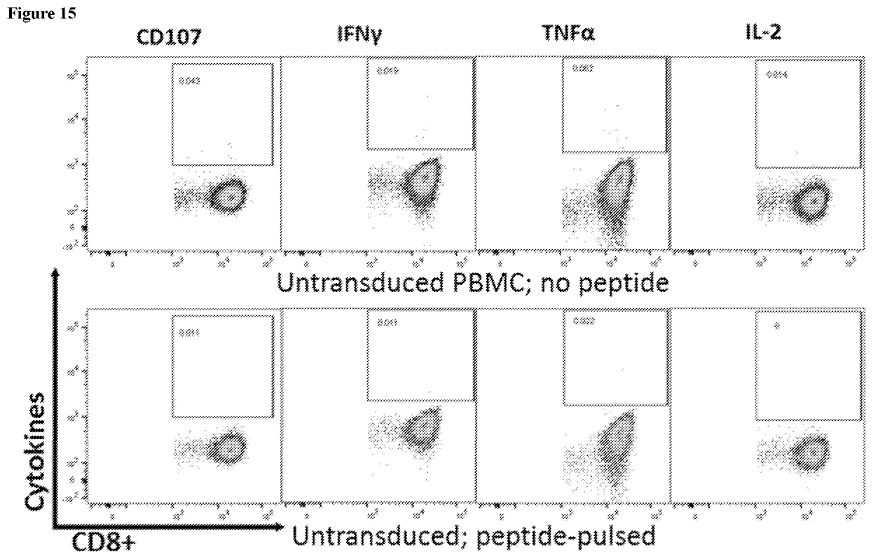

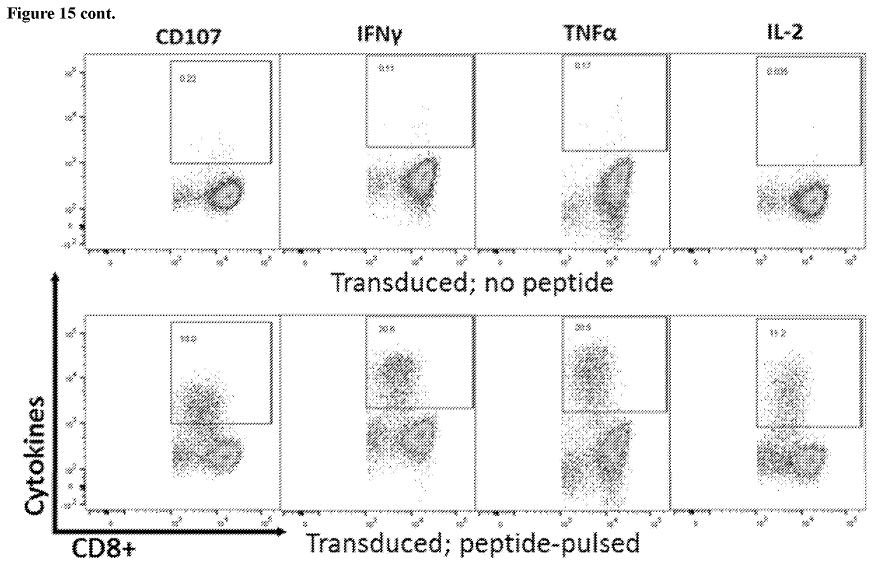

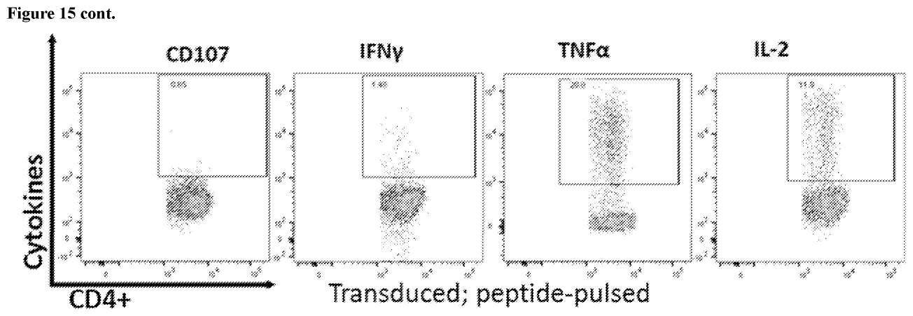

Data demonstrate successful transduction of E5-NLD-TCR in PBMCs (see FIG. 14). In addition, transduced CD8⁺ T cells showed higher CD107, IFNγ, TNFα and IL-2 expression compared to nontransduced cells stimulated with the same peptide (see FIG. 15). Transduced CD4⁺ T cells displayed relatively comparable TNFα and IL-2 expression to CD8⁺ T cells. However, IFNγ and CD107 are notably lower in CD4⁺ T cells, which suggest the contribution of CD8 co-receptor in target binding.

### Example 7: T Cells Engineered to Express a TCR from E5 Antigen (E5-NLD-TCR) Displayed High Functional Avidity (Secondary Screening)

Transduced and un-transduced T cells were stimulated with HLA-matched LCL pulsed with different concentrations of cognate antigenic peptide (i.e., (10⁻⁶, 10⁻⁷, 10⁻⁸, 10⁻⁹, 10⁻¹⁰, 10⁻¹¹, 10⁻¹² and 10⁻¹³ mole/L)) for 4 hours in R10 media containing monensin and brefeldin at 37° C., 6.5% CO₂. Cells were then washed and the pellet re-suspended in wash buffer containing FITC-conjugated anti-CD4 and PerCP-Cy5.5-conjugated anti-CD8 antibodies. Following incubation with conjugated antibodies at 4° C. for 30 minutes, cells were washed, fixed, and permeabilized for 20 min. Cells were then washed and incubated with PE-anti-IFNγ for 4° C. for 30 minutes. The stained cells were further washed and re-suspended in PBS containing 1% paraformaldehyde and analyzed by flow cytometry.

Results

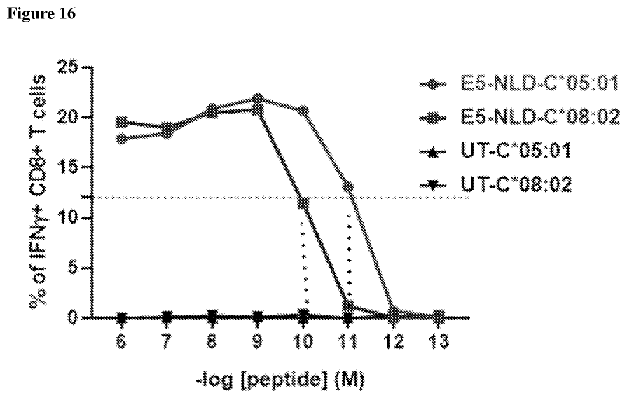

As shown in FIG. 16, IFNγ expression was induced in E5-NLD-lenti-transduced PBMC stimulated with HLA-matched (C*05:01 and C*08:02), pulsed LCL. Notably, CD8⁺ T cells showed recognition of LCL pulsed with as little as 10 pmol/L NLD peptide (SEQ ID NO. 1); essentially demonstrating functional avidity greater than that of TCR gene-engineered T cells used in other gene therapy trials that have mediated tumor regression (Draper et al., Clin Cancer Res 21: 4431-4439, 2015; Doran et al., Journal of Clinical Oncology 2019 37:30, 2759-2768).

### Example 8: T Cells Engineered to Express a TCR from E2 Antigen (E5-TLQ-TCR) Specifically Recognized and Killed HLA-A2⁺ HPV-16⁺ Tumor Cells. (In Vitro Cytolysis)

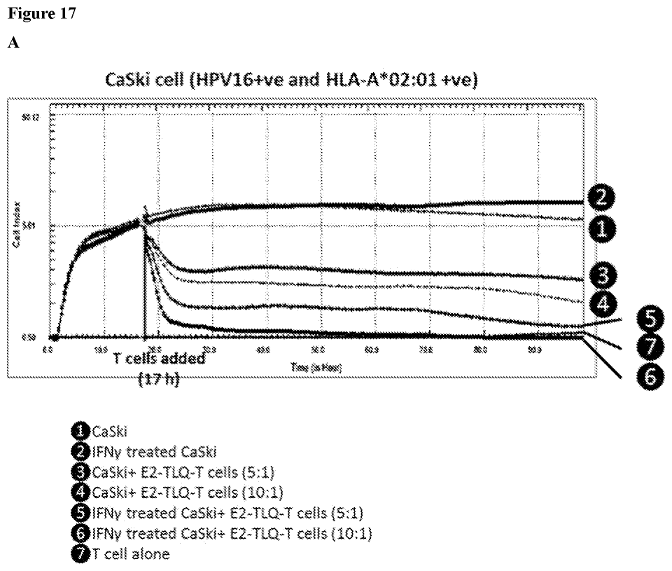

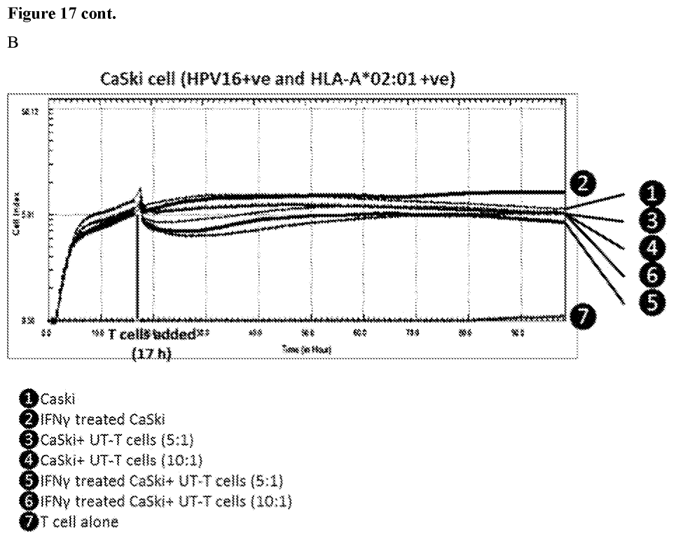

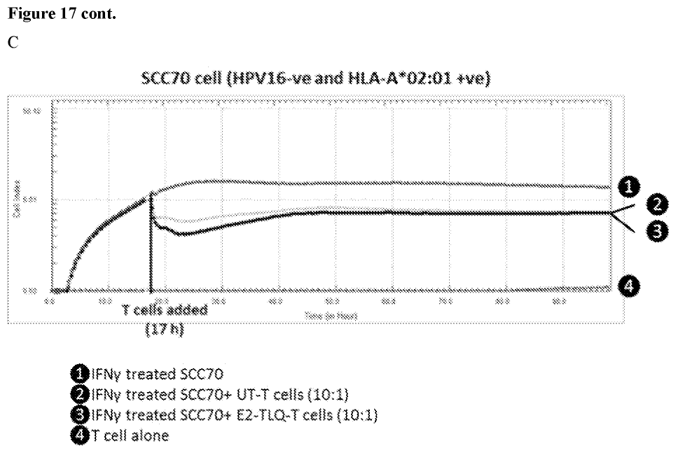

E2-TLQ-TCR-T cell-mediated cytolysis of tumor cell lines at effector-to-target (E/T) ratios of 10:1 and 5:1. Briefly, 2×10⁴ target cells per well (CaSki (HPV⁺) and SCC70 (HPV−)) were seeded and cultured overnight (17 hours). Effector T cells (E2-TLQ-TCR-T cells) were added at the indicated ratios (see FIG. 17) when the cell index approached plateau and co-cultured for approximately 4 days to assess cytolysis over time. IFNγ treated (100 μ/ml) target cells were also included as control; IFNγ being added to the target cell culture for 24 hours and the culture washed with complete RPMI media prior to assay.

Target cell lysis was evaluated by real time cell analysis through electrical impedance of adherent cells in each well every 15 minutes, until the end of the experiment. Results are reported as a cell index value.

The CaSki cell line (HPV16⁺; HLA-A*02:01⁺) was challenged with E2-TLQ-TCR-T cells (FIG. 17, A) and with “untransduced” control T cells (FIG. 17, B). E2-TLQ-TCR-T cells (HLA-A*02:01 restricted) induced cytolysis of the HPV16⁺ cancer cell line (CaSki) as indicated by the drop in cell index following addition of E2-TLQ-TCR-T cells. Greater cytolysis of IFNγ-treated target (CaSki) cells, relative to untreated, was observed and may be related to enhanced antigen processing and expression of MHC molecules. However untransduced T cells showed no cytolysis when added at the same effector-to-target ratio.

Control cell line SCC70 (HPV16⁻ and HLA-A*02:01⁺) was challenged with both E2-TLQ-TCR T cells and untransduced T cells (UT). E2-TLQ-T cells induce antigen-specific cytolysis as no cytolysis was observed in the HPV16⁻ cell line (SCC70) at 10:1 effector-to-target ratio. (FIG. 17, C)

### INCORPORATION BY REFERENCE

All publications, patents, patent applications and sequence accession numbers mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

A number of embodiments of the invention have been described. Nevertheless, it will be understood that various modifications may be made without departing from the spirit and scope of the invention. Accordingly, other embodiments are within the scope of the following claims. Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

What is claimed is:

1. A T cell receptor (TCR) polypeptide having antigenic specificity for human papillomavirus (HPV) 16, wherein the TCR comprises at least one complementary determining region 3α (CDR3α) amino acid sequence and at least one CDR3β amino acid sequence selected from the amino acid sequences set forth in SEQ ID NOs. 13 to 52, or 219-226.

2. A TCR polypeptide having antigenic specificity for HPV16, wherein the TCR is specific for antigens comprising at least one epitope having an amino acid sequence selected from the amino acid sequences set forth in SEQ ID NOs: 1-12, 217 or 218.

3. The TCR polypeptide of claim 1 or 2, wherein the TCR has antigenic specificity for HPV16 peptides other than E6 and E7.

4. The TCR polypeptide of any one of claims 1 to 3, wherein the TCR has antigenic specificity for any one of HPV16 peptides E1, E2, E4, or E5.

5. The TCR polypeptide of any one of claims 1 to 4, wherein the TCR has antigenic specificity for antigens comprising at least one epitope having an amino acid sequence selected from the amino acid sequences set forth in SEQ ID NOs. 1-7.

6. The TCR polypeptide of claim 4, wherein the TCR comprises a CDR3α amino acid sequence and CDR3β amino acid set forth in SEQ ID NOs. 13 and 14, SEQ ID NOs. 25 and 26, or SEQ ID NOs. 51 and 52.

7. The TCR polypeptide of claim 4, wherein the TCR comprises a TCRβ amino acid sequence and TCRα chain amino acid set forth in SEQ ID NOs. 59 and 60 or SEQ ID NOs. 61 and 62.

8. A nucleic acid encoding the TCR polypeptide of any one of claims 1 to 7.

9. The nucleic acid of claim 8, comprising any one of the nucleic acid sequences set forth in SEQ ID NOs. 53 to 58, 227, 228, and/or fragments thereof.

10. An isolated nucleic acid comprising a nucleotide sequence encoding one or more polypeptides comprising a TCR comprising an amino acid sequence selected from SEQ ID NOs. 59 to 70, 229-232, or 209 to 216.

11. The nucleic acid of any one of claims 8 to 10, wherein the nucleic acid is an expression vector.

12. The nucleic acid of claim 11, wherein the expression vector is a viral vector.

13. The nucleic acid of claim 12, wherein the viral vector is lentiviral expression vector.

14. An immune cell comprising the nucleic acid of any one of claims 8 to 13.

15. An immune cell expressing the TCR polypeptide of any one of claims 1 to 6.

16. The immune cell of claim 14 or 15, wherein the immune cell is a leukocyte.

17. The immune cell of any one of claims 14 to 16 wherein the immune cell is a lymphocyte, a monocyte, a macrophage, a dendritic cell, a mast cell, a neutrophil, a basophil, or an eosinophil.

18. The immune cell of any one of claims 14 to 17 wherein the immune cell is a lymphocyte selected from an αβT cell, γδT cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

19. The immune cell of claim 18, wherein the immune cell is a cytotoxic T lymphocyte (CTL).

20. The immune cell of claim 18 or 19, wherein the immune cell is a viral antigen-sensitized CTL.

21. The immune cell of any one of claims 18 to 20, wherein the immune cell is a CTL sensitized to a viral antigen from any one of human papilloma virus (HPV), Epstein-Barr virus (EBV), cytomegalovirus (CMV), B.K. virus (BKV), John Cunningham virus (JCV), picornavirus (e.g., Hepatitis A virus), hepadnavirus (e.g., Hepatitis B virus), hepacivirus (e.g., Hepatitis C virus), deltavirus (e.g., Hepatitis D virus), hepevirus (e.g., Hepatitis E virus), or any combination thereof.

22. The immune cell of any one of claims 18 to 21, wherein the immune cell is an HPV-sensitized CTL.

23. The TCR-expressing cell of any one of claims 15 to 22, wherein the TCR-expressing cell is genetically modified to no longer express one or more immune checkpoint molecules.

24. The TCR-expressing cell of any one of claims 15 to 22, wherein said cell also expresses a dominant-negative form of one or more immune checkpoint molecules.

25. The TCR-expressing cell of any one of claims 15 to 22, wherein said cell also expresses switch receptors specific for one or more immune checkpoint molecules.

26. The TCR-expressing cell of any one of claims 15 to 22, wherein said cell also expresses antibodies, or functional fragments thereof, capable of blocking signaling by one or more checkpoint molecules.

27. The TCR-expressing cell of any one of claims 21 to 26, wherein the immune checkpoint molecules are selected from programmed death 1 (PD-1), programmed death-ligand 1 (PD-L1), programmed death-ligand 2 (PD-L2), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), CD160, and transforming growth factor β (TGF-α) receptor.

28. The immune cell of any one of claims 23 to 27, wherein the immune checkpoint molecule is PD-1 and/or CTLA-4.

29. A method of treating a HPV-associated cancer or precancerous lesions in a subject, the method comprising administering an effective amount of an adoptive immunotherapy composition comprising the TCR-expressing cells of any one of claims 14 to 28.

30. The method of claim 29, wherein the HPV-associated cancer is a squamous cell carcinoma.

31. The method of claim 29 or 30, wherein the HPV-associated cancer is selected from head and neck cancer (e.g., HNSCCs and the like) and cancers of the cervix, anus, vagina, vulva, penis, tongue base, larynx, and tonsil.

32. The method of claim 28, wherein the HPV-associated precancerous lesion comprises abnormal cell changes and/or precancerous cell changes selected from: cervical intraepithelial neoplasia (CIN), squamous intraepithelial lesions (SIL), or warts on the cervix.

33. The method of any one of claims 29 to 32, wherein the subject has received, is receiving, or will receive an additional anti-cancer therapy.

34. The method of claim 33, wherein the additional anti-cancer therapy comprises surgery, radiation, chemotherapy, immunotherapy, or hormone therapy.

35. The method of claim 34, wherein the subject has received IFNγ prior to administering the adoptive immunotherapy composition.

36. The method of any one of claims 29 to 35, wherein the adoptive immunotherapy composition is administered intrapleurally, intravenously, subcutaneously, intranodally, intratumorally, intrathecally, intraperitoneally, intracranially, or by direct administration to an organ.

37. The method of any one of claims 29 to 36, wherein the subject is human.

38. The method of claim 37, wherein the TCR-expressing cells of the adoptive immunotherapy composition are derived from the subject.

39. The method of claim 37, wherein the TCR-expressing cells of the adoptive immunotherapy composition are derived from a donor sample, or from a bank or library of donor samples.

40. The method of any one of claims 29 to 39, further comprising administering at least one immune checkpoint inhibitor.

41. The method of claim 40, wherein the immune checkpoint inhibitor comprises an anti-PD-1 antibody, anti-PD-L1 antibody, anti-PD-L2 antibody, anti-CTLA-4 antibody, or a combination thereof.

42. The method of claim 40, wherein the immune checkpoint inhibitor comprises an RNAi molecule such as an antisense RNA molecule (asRNA), micro RNA molecule (miRNA), short hairpin RNA molecule (shRNA), or small interfering RNA molecule (siRNA).

43. The method of claim 40, wherein the immune checkpoint inhibitor comprises a CRISPR RNA (crRNA) molecule.

44. The method of claim 40, wherein the immune checkpoint inhibitor comprises a dominant-negative form of an immune checkpoint molecule.

45. The method of claim 40, wherein the immune checkpoint inhibitor comprises a recombinant switch receptor.

46. The method of any of claims 40 to 45, wherein immune checkpoint inhibitor is expressed by a vector comprising a nucleic acid molecule encoding the immune checkpoint inhibitor, wherein the vector is selected from a DNA vector, an RNA vector, a plasmid, or a viral vector.

47. The method of claim 46, wherein the vector comprising a nucleic acid molecule encoding the immune checkpoint inhibitor is expressed in the TCR-expressing cells of the adoptive immunotherapy composition.

48. A cell bank of comprising cells for adoptive immunotherapy, wherein the cells are the TCR-expressing cells of any one of claims 14 to 28, and wherein the HLA restriction of the TCR-expressing cells is known.

49. A method of treating HPV-associated cancer or precancerous lesions in a subject, the method comprising administering an effective amount of an adoptive immunotherapy composition comprising TCR-expressing cells selected from the cell bank of claim 48.
